SEQUENCE LISTING (64 sequences)

SEQ ID NO: 1 - DNA, 18 bp, Artificial Sequence
  I-SceI nuclease recognition site
tagggataacagggtaat

SEQ ID NO: 2 - DNA, 60 bp, Artificial Sequence
  Primer
gaaattaatacgactcactataggatgaatgttcgggtacttctgttttagagctagaaa

SEQ ID NO: 3 - DNA, 60 bp, Artificial Sequence
  Primer
gaaattaatacgactcactataggcggtgcggccgcataggcgcgttttagagctagaaa

SEQ ID NO: 4 - DNA, 25 bp, Artificial Sequence
  Primer
tgtgagtagattccttcgtcgttgg

SEQ ID NO: 5 - DNA, 25 bp, Artificial Sequence
  Primer
attgcgtagcaagtttaccttgggc

SEQ ID NO: 6 - DNA, 25 bp, Artificial Sequence
  Primer
agcaaagtgaacacgtcgctaagcg

SEQ ID NO: 7 - DNA, 26 bp, Artificial Sequence
  Primer
tcaccttcagcttcacggtgttgtgg

SEQ ID NO: 8 - DNA, 23 bp, Artificial Sequence
  Primer
ttcttcaagaccaggcctcaatc

SEQ ID NO: 9 - DNA, 25 bp, Artificial Sequence
  Primer
tcactaaactcagccagtatcctat

SEQ ID NO: 10 - DNA, 22 bp, Artificial Sequence
  Primer
acgaccgcatacaaaacgtacg

SEQ ID NO: 11 - DNA, 19 bp, Artificial Sequence
  Primer
tcgtactgctccacgatgg

SEQ ID NO: 12 - DNA, 26 bp, Artificial Sequence
  Primer
aatcagccataccacatttgtagagg

SEQ ID NO: 13 - DNA, 31 bp, Artificial Sequence
  Primer
aatcatgggtaggacgaatgtcttactcagc

SEQ ID NO: 14 - DNA, 33 bp, Artificial Sequence
  Primer
ttttggtaccgccagatcgcagatagagtgtgc

SEQ ID NO: 15 - DNA, 33 bp, Artificial Sequence
  Primer
ttttaccggtacccgaacattcatctttatttc

SEQ ID NO: 16 - DNA, 38 bp, Artificial Sequence
  Primer
ttttcctaggcggccgctaaaataaacaacattatcag

SEQ ID NO: 17 - DNA, 35 bp, Artificial Sequence
  Primer
ttttcctagggttggctctctatttgcactccacc

SEQ ID NO: 18 - DNA, 35 bp, Artificial Sequence
  Primer
gtcaacgcgtggatcactatcaaacccctaaggac

SEQ ID NO: 19 - DNA, 38 bp, Artificial Sequence
  Primer
gtcaggatccagacatcctctagatttgttcgttgatc

SEQ ID NO: 20 - DNA, 32 bp, Artificial Sequence
  Primer
gtcaggatccatgcccaagaagaagcgcaagg

SEQ ID NO: 21 - DNA, 35 bp, Artificial Sequence
  Primer
gtcagtcgacttatttcaggaaagtttcggaggag

SEQ ID NO: 22 - DNA, 37 bp, Artificial Sequence
  Primer
gtcagcggccgctctagacgtaatcgaagtgttggac

SEQ ID NO: 23 - DNA, 41 bp, Artificial Sequence
  Primer
gtcagaattcctcgagcgcccttttcgtcataaaatcgtag

SEQ ID NO: 24 - DNA, 30 bp, Artificial Sequence
  Primer
aagacgatgagttctactggcgtggaatcc

SEQ ID NO: 25 - DNA, 30 bp, Artificial Sequence
  Primer
cttgccgtatgtgatggagcgttgtcatgg

SEQ ID NO: 26 - DNA, 30 bp, Artificial Sequence
  Primer
ttgtttactctcagtgcagtcaacatgtcg

SEQ ID NO: 27 - DNA, 30 bp, Artificial Sequence
  Primer
ttcgacagtcaaggttgacacttcacaagg

SEQ ID NO: 28 - DNA, 249 bp, Artificial Sequence
  Flanking Sequence
gatccaatgctgaggaaattacaaatgttttttcagtgtgttttttgaaaaatgtcacaactttaagttaaagtttagtatacaaaattcaaaaaatgtttttggaaaaatacatacgaagtaagaataactctttgcctttcgaatgcggcttagagagtttcattagacgcgtaatcacagagatatggacagaacacttttgtatgttgttgagggggtgaacccaaacttttgcacgggagtgta

SEQ ID NO: 29 - DNA, 241 bp, Artificial Sequence
  Flanking Sequence
tatatggcgaatacagaacgaaactacatcttgaaatttgaaaagaacaaaattgtgaatcgaacagccttgaatgtttagaatttgattgggtacttattcgtctgtagtgctttttatctctctctctgctgatgcataatttgaacgcataacgactttcagacttcacaagttctagcaacatttcaacttataatcgttgaaaagtatacaacattaagatttcaaaatgatgatc

SEQ ID NO: 30 - DNA, 29 bp, Artificial Sequence
  Flanking Sequence
gatccagaatgagcaaagtgtcactttta

SEQ ID NO: 31 - DNA, 208 bp, Artificial Sequence
  Flanking Sequence
tacttgcggaataattgagcggaacatttttccgtacggaatagtgacagctccatttgatttgtacagcaggcgttaccaatgttacgaaatcagctctacttgtcaactggatacagttcaagtaatttgaacagctgaagtatttcttgaccattacttgtcctatccttttgcacagtacttacgaagtggataccaaccatta

SEQ ID NO: 32 - DNA, 23 bp, Artificial Sequence
  Single Guide RNA
atgaatgttcgggtacttctcgg

SEQ ID NO: 33 - DNA, 23 bp, Artificial Sequence
  Single Guide RNA
cggtgcggccgcataggcgcggg

SEQ ID NO: 34 - DNA, 50 bp, Artificial Sequence
  Coding Sequence
cggtcgccaccatgggtagggataacagggtaattagggtgcgctcctcc

SEQ ID NO: 35 - DNA, 46 bp, Artificial Sequence
  Coding Sequence
cggtcgccaccatgggtagggcagggtaattagggtgcgctcctcc

SEQ ID NO: 36 - DNA, 65 bp, Artificial Sequence
  Coding Sequence
gggatccaccggtcgccaccatgggtagggataacagggtaattagggtgcgctcctccaagaac

SEQ ID NO: 37 - DNA, 64 bp, Artificial Sequence
  Coding Sequence
gggatccaccggtcgccaccatgggtaggataacagggtaattagggtgcgctcctccaagaac

SEQ ID NO: 38 - DNA, 60 bp, Artificial Sequence
  Coding Sequence
gggatccaccggtcgccaccattattattattagtaattagggtgcgctcctccaagaac

SEQ ID NO: 39 - DNA, 73 bp, Artificial Sequence
  Coding Sequence
gggatccaccggtcgccaccatgggtaggtaattagggtaattagggtaattagggtgcgctcctccaagaac

SEQ ID NO: 40 - DNA, 53 bp, Artificial Sequence
  Coding Sequence
gggatccaccggtcgccaccatgggtgtaattagggtgcgctcctccaagaac

SEQ ID NO: 41 - DNA, 53 bp, Artificial Sequence; identical to SEQ ID NO: 40
  Coding Sequence

SEQ ID NO: 42 - DNA, 60 bp, Artificial Sequence
  Coding Sequence
gggatccaccggtcgccaccatgggataacagggtaattagggtgcgctcctccaagaac

SEQ ID NO: 43 - DNA, 60 bp, Artificial Sequence; identical to SEQ ID NO: 42
  Coding Sequence

SEQ ID NO: 44 - DNA, 53 bp, Artificial Sequence; identical to SEQ ID NO: 40
  Coding Sequence

SEQ ID NO: 45 - DNA, 64 bp, Artificial Sequence
  Coding Sequence
gggatccaccggtcgccaccatgggtagggataaagggtaattagggtgcgctcctccaagaac

SEQ ID NO: 46 - DNA, 53 bp, Artificial Sequence; identical to SEQ ID NO: 40
  Coding Sequence

SEQ ID NO: 47 - DNA, 64 bp, Artificial Sequence; identical to SEQ ID NO: 37
  Coding Sequence

SEQ ID NO: 48 - DNA, 63 bp, Artificial Sequence
  Coding Sequence
gggatccaccggtcgccaccatgggtagggataagggtaattagggtgcgctcctccaagaac

SEQ ID NO: 49 - DNA, 64 bp, Artificial Sequence; identical to SEQ ID NO: 37
  Coding Sequence

SEQ ID NO: 50 - DNA, 57 bp, Artificial Sequence
  Coding Sequence
gggatccaccggtcgccaccatgggtaggggtaattagggtgcgctcctccaagaac

SEQ ID NO: 51 - DNA, 57 bp, Artificial Sequence; identical to SEQ ID NO: 50
  Coding Sequence

SEQ ID NO: 52 - DNA, 66 bp, Artificial Sequence
  Coding Sequence
gggatccaccggtcgccaccatgggtagggataattagggtaattagggtgcgctcctccaagaac

SEQ ID NO: 53 - DNA, 66 bp, Artificial Sequence; identical to SEQ ID NO: 52
  Coding Sequence

SEQ ID NO: 54 - DNA, 63 bp, Artificial Sequence
  Coding Sequence
gggatccaccggtcgccaccatgggtagataacagggtaattagggtgcgctcctccaagaac

SEQ ID NO: 55 - DNA, 61 bp, Artificial Sequence
  Coding Sequence
gggatccaccggtcgccaccatgggtataacagggtaattagggtgcgctcctccaagaac

SEQ ID NO: 56 - DNA, 61 bp, Artificial Sequence; identical to SEQ ID NO: 55
  Coding Sequence

SEQ ID NO: 57 - DNA, 60 bp, Artificial Sequence; identical to SEQ ID NO: 42
  Coding Sequence

SEQ ID NO: 58 - DNA, 64 bp, Artificial Sequence; identical to SEQ ID NO: 37
  Coding Sequence

SEQ ID NO: 59 - DNA, 60 bp, Artificial Sequence; identical to SEQ ID NO: 42
  Coding Sequence

SEQ ID NO: 60 - DNA, 61 bp, Artificial Sequence
  Coding Sequence
gggatccaccggtcgccaccatgggtagggcagggtaattagggtgcgctcctccaagaac

SEQ ID NO: 61 - DNA, 53 bp, Artificial Sequence; identical to SEQ ID NO: 40
  Coding Sequence

SEQ ID NO: 62 - DNA, 63 bp, Artificial Sequence
  Coding Sequence
gggatccaccggtcgccaccgtgggtaagatacagggtaattagggtgcgctcctccaagaac

SEQ ID NO: 63 - DNA, 65 bp, Artificial Sequence; identical to SEQ ID NO: 36
  Coding Sequence

SEQ ID NO: 64 - DNA, 65 bp, Artificial Sequence; identical to SEQ ID NO: 36
  Coding Sequence

(19) United States
(12) Patent Application Publication
(10) Pub. No.: US 2023/0242900 A1
(43) Pub. Date: Aug. 3, 2023

(54) SELF-ELIMINATING TRANSGENES

(71) Applicant: The Texas A&M University System, College Station, TX (US)

(72) Inventors: Zach N. Adelman, College Station, TX (US); Kevin M. Myles, College Station, TX (US); Sakiko Okumoto, College Station, TX (US)

(21) Appl. No.: 18/015,753

(22) PCT Filed: Jul. 16, 2021
(86) PCT No.: PCT/US21/41951

§ 371 Date: Jan. 12, 2023

Related U.S. Application Data
(60) Provisional application No. 63/052,800, filed on Jul. 16, 2020.

(51) Int. Cl. C12N 15/10; C12N 15/82; C12N 15/85

(52) U.S. Cl. CPC C12N 15/102; C12N 15/8213; C12N 15/8509; C12N 2310/20

(57) ABSTRACT

The current invention provides vector constructs that are pre-programmed to self-terminate or self-remove at a predetermined time and methods of making the same. The present invention further provides methods for creating organisms containing these vector constructs. Also provided are various transgenic organisms with the vector constructs, including plants, insects, and mammals.

### REFERENCE TO RELATED APPLICATION

This application claims the benefit of U.S. Provisional Application No. 63/052,800, filed Jul. 16, 2020, which is herein incorporated by reference in its entirety.

### STATEMENT REGARDING FEDERALLY SPONSORED RESEARCH

This invention was made with Government support under Grant No. HR0011-16-2-0036 awarded by the Defense Advanced Research Projects Agency (DARPA) of the U.S. Department of Defense and under Vector Biology Grant No. 1R01AI148787-01A1 awarded by the National Institutes of Health. The Government has certain rights in the invention.

### INCORPORATION BY REFERENCE OF SEQUENCE LISTING

The present application includes a Sequence Listing which has been submitted in ASCII format via EFS-Web and is hereby incorporated by reference in its entirety. Said Sequence Listing, created on Jul. 15, 2021, is named TAMC054WO_ST25.txt and is 13.8 kilobytes in size.

### FIELD OF THE INVENTION

The present invention relates to the fields of biotechnology, molecular biology, and genetics. More specifically, the invention relates to vector constructs that are pre-programmed to self-terminate, or self-eliminate, at a predetermined time or under a pre-determined set of conditions.

### BACKGROUND OF THE INVENTION

There is concern for the spread of unwanted transgenic sequences into nature. Gene drive systems have tremendous potential for application across a wide range of biotechnology-related fields, including the potential to control vector-borne diseases, or invasive and unwanted species, as well as other agricultural, synthetic biology, and human medicine applications. Such gene drive systems and their use are known in the art, although the use of these systems is often complicated by the need to remove or reverse the genes introduced into a population through these gene drive systems as well as the functional gene drive elements. There is therefore a need in the art to enable removal or reversal of such genes and functional gene drive elements.

### SUMMARY

In some aspects, provided is a recombinant polynucleotide construct including direct repeat sequences flanking a DNA sequence that includes a transgene and at least a first site-specific nuclease recognition site. In some embodiments, the DNA sequence includes a first site-specific nuclease recognition site and a second site-specific nuclease recognition site flanking the transgene. In further embodiments, the first and second site-specific nuclease recognition site are the same. In yet further embodiments, the first and second site-specific nuclease recognition site are different. In some aspects, the site-specific nuclease recognition site is recognized by an engineered nuclease. In other aspects, the site-specific nuclease recognition site is recognized by a nuclease native to at least a first eukaryotic species.

In further embodiments, the DNA sequence includes a reporter gene. In some embodiments, the direct repeat sequences include from about 2 to about 200 repeats. In other embodiments, the direct repeat sequences include from about 15 to about 20,000 nucleotides. In yet further embodiments, the polynucleotide construct includes a selectable marker. In other aspects, the polynucleotide construct includes a nucleic acid sequence encoding a nuclease that recognizes the site-specific nuclease recognition site. In further embodiments, the nucleic acid sequence is operably linked to an inducible or tissue-specific promoter. In yet further embodiments, the tissue-specific promoter is a germline-specific promoter. In yet further embodiments, the polynucleotide construct includes a second nucleic acid sequence encoding a second nuclease that recognizes a second site-specific nuclease recognition site in the DNA sequence. In further embodiments, the first and second nucleic acid sequences are operably linked to different promoters that drive different levels of expression.

In yet another aspect, provided are host cells that include the polynucleotide constructs described herein. In further embodiments, provided are transgenic plants, insects or non-human animals that include the polynucleotide constructs described herein, wherein the transgene is capable of being eliminated in the progeny of the plants, insects or non-human animals. In further embodiments, the host cell is a plant, insect, non-human animal, or human cell.

In yet another aspect, provided is a method of transforming a host cell including introducing the polynucleotide constructs described herein into the cell. In further embodiments, provided is a method of eliminating a transgene sequence from a cell by subjecting a cell that has been transformed to include the polynucleotide constructs described herein to an external stimulus that causes the transgene sequence to be eliminated. In further embodiments, the external stimulus is a chemical stimulus.

In a further aspect, provided is a recombinant polynucleotide construct including recombination sites flanking a DNA sequence that includes a transgene, such as for instance, a transgene for gene drive, and at least a first DNA sequence encoding a recombinase recognizing the recombination sites.

In still a further aspect, provided is a recombinant polynucleotide construct including inverted terminal repeats flanking a DNA sequence that includes a transgene, such as for instance, a transgene for gene drive, and at least a first DNA sequence encoding an integration-deficient transposase recognizing the inverted terminal repeats.

### BRIEF DESCRIPTION OF THE DRAWINGS

FIG. 1: Shows a diagram of a self-decaying gene drive system with a fast homology-dependent repair (“HDR”)-mediated gene drive and slow single-strand annealing (“SSA”)-mediated self-decay. Two site-specific nucleases (upper left) are expressed in unequal quantities. DNA break induction by the first nuclease on the opposite chromosome is followed by homology-based repair increasing transgene copy number and resulting in gene drive. Lower expression of the second nuclease results in low level DNA break induction specifically in the inserted transgene; repair via the SSA pathway results in complete loss of all transgene sequence. Black bars indicate tandem duplicated sequences that drive SSA-based repair.

FIG. 2: Shows upstream activating sequence (“UAS”)-driven or tetOff-controlled nuclease expression constructs to trigger transgene self-elimination.

FIG. 3: Shows a diagram of parameters for optimization. Labeled parameters affect the rate of SSA-based repair of dsDNA breaks (self-decay). The length of the direct repeats (X), distance between repeat and DNA break (Y) and distance between repeats (Z) are expected to contribute to SSA efficiency.

FIG. 4: Shows constructs for evaluating the self-eliminating transgene in Tribolium. Shown are direct repeats (DR) flanking a fluorescent marker (enhanced green fluorescent protein, “EGFP”) following by either a heat-inducible promoter (hsp70) or the bipartite tetO system to control nuclease expression. Nuclease activation (homing endonuclease genes, “HEG”) triggers DNA break induction and repair using SSA to eliminate all transgenic sequences.

FIG. 5: Shows a construct for evaluating the self-eliminating transgene in A. palmeri. SSA-based repair between the two recognition sites results in the loss of the transgenes. Activities of DNA repair through NHEJ or HR-based repairs can be measured by the distance between repeats, detected by a PCR reaction using a set of transgene specific primers (indicated by arrowheads).

FIG. 6A and FIG. 6B: Show two types of repair events that were recovered in a prior study using HEGs to introduce double stranded DNA breaks in the Ae. aegypti germline: non-homologous end-joining (“NHEJ”) following cutting at each homing endonuclease (“HE”) recognition site flanking the EGFP gene (Y2-I-Anil only) and SSA-based repair following cutting a one HE site (I-SceI, I-CreI, Y2-I-Anil).

FIG. 7: Shows the parameters to be analyzed predicted to affect the rate of SSA-based repair of dsDNA breaks (selfelimination). The length of the direct repeats (DR), distance between repeat and DNA break (spacer).

FIG. 8: Shows transgene insertion in the D. melanogaster yellow (y) gene (black and grey boxes), with direct repeats (DR) and ISceI target site indicated.

FIG. 9: Shows increasing the length of direct repeats results in concomitant increases in SSA, after providing ISceI from a plasmid source.

FIG. 10: Shows a representation of the transgene insertion in the Ae. aegypti kmo gene (black and grey boxes), with 700 bp direct repeats (DR) indicated, along with the I-SceI target site (top panel) and a Table of the results showing Ae. aegypti larvae containing the starting transgene and expressing both EGFP and DsRed (WGR), after losing DsRED expression due to NHEJ (WG), or after losing both DsRED and EGFP markers and regaining eye pigmentation following SSA-based transgene elimination (B) (lower panel).

FIG. 11: Shows a representation of a transgene flanked by direct repeats (DR), with DSB sites (red) and distances to the near/far repeats (blue/purple) indicated.

FIG. 12: Shows sgRNAs (arrows) targeting each transgene. Representation of transgenes already integrated into the Drosophila (y-G, y-ISE) and Ae. aegypti (kmoRG) genome. Arrows indicate potential sgRNA groups.

FIG. 13: Shows ϕC31-mediated insertion of UAS.hsp70.I-SceI into y-G fly lines. Panel A shows steps 1 and 2 that generated multiple transgenic D. melanogaster lines containing the y-ISE construct with various direct repeat lengths. Panel B shows the yellow gene drive construct (y-MCR) that will be recombined into existing y-ISE lines through strategically placed attP.attB sites (steps 3 and 4) permitting elimination of an active gene drive from a population of insects.

FIG. 14: Shows self-elimination of a transgene. Heat shock (HS) of the y-ISE 250DR strain (grey dots) resulted in SSA-based elimination of the transgene, at significantly higher levels than were observed in control y-G flies (green dots), lacking the UAS.hsp70.I-SceI element. Precise elimination of the transgene in y-ISE flies was confirmed by the presence of a single nucleotide scar (TGG→TcG). Each dot represents an individual replicate experiment with n≥80. Statistical significance determined by Wilcoxon test and indicated by * (ns=not significant).

FIG. 15: Shows modeling a Self-eliminating gene drive. Proportion of transgene-free alleles after a single simulated release of individuals at 1% of a wild-type population after 60 generations for a gene drive targeting yellow (panel A) or DSX (panel B). Stars indicate rates of self-elimination already obtained, well within the range of predicted effectiveness (left of white line without numbers).

FIG. 16: Shows programmable self-elimination of a self-sustaining dsx gene drive. Panel A shows male- and female-specific transcripts of the D. melanogaster doublesex (dsx) gene. Shaded boxes represent coding sequences, while white boxes represent untranslated regions, straight lines represent introns, and bent lines represent splice acceptor sites. Panel B shows target site for CRISPR/Cas9-based gene drive targeting D. melanogaster doublesex gene.

FIG. 17: Shows homology-based gene insertion of a gene drive transgene into the Ae. aegypti kmo locus. The recipient strain (top) has been developed and validated site-specific insertion with two kmo^{RG} constructs (200+700 bp direct repeats). The donor constructs will be developed using the exact same homology arms and sgRNA.

FIG. 18: Mechanisms for a self-eliminating CRISPR/Cas9-based gene drive (GD). The GD transgene is linked to Marker (M) and Cargo (C) genes, with the self-elimination mechanism based on: (Panel A) a site-specific recombinase (REC) and corresponding recombination (R) sites, (Panel B) an integration-defective transposase (TE) and corresponding inverted terminal repeats (ITR), or (Panel C) single-strand annealing (SSA)-based DNA repair initiated by a nuclease (NUC) and enabled by direct repeats (DR). In all cases, the disrupted, non-functional host gene is indicated by white boxes, with the restored, functional gene indicated by filled boxes at the bottom of each panel. Vertical bars indicate the recoded sequences rendering the restored gene resistant to the GD.

FIG. 19: Modelling a self-eliminating gene drive. (Panel A) Six different allele types considered in the deterministic model. Two alleles contain the gene drive (GD), and either functional (g) or defective (s) self-elimination mechanism (SEM). Other alleles include wild-type, CRISPR-susceptible (w), wild-type, pre-determined CRISPR-resistant (v), CRISPR-resistant no cost (u), and CRISPR-resistant high cost (r). GD, gene drive; SE, self-elimination gene; M, marker gene; C, cargo gene. (Panel B) Structure and probabilities associated with the deterministic model and their relation to the six allele types: a, probability that self-elimination occurs; (3, probability that the transgene is not altered by the self-elimination mechanism; γ, probability that the self-elimination mechanism breaks down without removing the transgene, with no chance for self-elimination to occur in any future generation; q, probability that the nuclease responsible for gene drive induces a double-stranded break at its target site on the homologous chromosome; p, the probability that the double-stranded break is repaired via homology-dependent repair (HDR).

FIG. 20: Self-elimination mechanisms accelerate the reversal of gene drive systems without intervention. (Panel A) Fitness penalties applied to each potential genotype for a non-essential gene. (Panel B) Proportion of transgene-free alleles after a single simulated release of gene drive containing individuals at 1% or 10% of a wild-type population when selection of no-cost resistance alleles is possible, at four self-elimination mechanism rates (α=0, 0.1, 0.4, 0.8), with a self-elimination mechanism failure rate of 1%.

FIG. 21: Transgene self-elimination mechanisms are predicted to tolerate high failure rates. All plots are based on a starting population of gene drive containing individuals at 1% of the population and show four rates of transgene elimination (α=0, 0.1, 0.4, 0.8) considering higher rates of failure of the self-elimination mechanism (0.05, 0.1).

FIG. 22: As resistance allele formation becomes more difficult, gene drive transgenes last progressively longer in a simulated population. Proportion of each allele in a simulated population after a single release of gene drive (genotype gg) individuals corresponding to 1% of the starting population. Probability δ was set to 0.33, 0.1, 0.01 or 0 to simulate the increasing likelihood that a random indel results in a non-functional gene product. Shaded panel is from FIG. 25A, but is included here for comparison purposes.

FIG. 23: Self-elimination strategies are predicted to remove a strong, sex-biasing gene drive and avert complete population elimination. Based on the approach previously described, female genotypes rr, gr, sr, gg, ss, gs have fitness cost of 100%. Male genotypes gr, sr, gg, ss, gs have fitness cost of 10% and male genotype rr has a fitness cost of 5%. The gene drive is considered to be active in both male and female germline with no chance of producing a functional resistance allele (δ=0). (Panel A) Proportion of transgene-free alleles (wt), absolute population size (Panel B), and allele frequencies (Panel C) after a single simulated release of gene drive (GD) individuals at 1% (top) or 10% (bottom) of a wild-type population at four different rates of transgene self-elimination (α=0, 0.1, 0.4, 0.8). For (Panels A+B), simulations using γ=0.01 and γ=0.1 are shown; For (Panel C), γ=0.01. Shaded panels are from FIG. 24B and are printed here for comparison purposes.

FIG. 24: Self-elimination mechanisms reverse potent gene drive systems. (Panel A) Fitness penalties applied in the simulation for each genotype for a homing-based gene drive system targeting a gene critical for female fertility. (Panel B) Proportion of transgene-free alleles after a single simulated release of gene drive containing individuals at 1% or 10% of a wild-type population when the selection for gene drive-resistant allele is not possible. Model outcomes for four self-elimination mechanism rates (α=0, 0.1, 0.4, 0.8) are shown, all include a self-elimination mechanism failure rate of 1%.

FIG. 25: Self-elimination strategies are predicted to provide temporal control of gene drive transgenes over a broad parameter space, even when natural resistance alleles cannot be selected. Proportion of each allele in a simulated population after a single release of gene drive (genotype gg) males corresponding to 1% of the starting population when no-cost CRISPR-resistance alleles (u) cannot form (δ=0) in the absence (Panel A) or presence of a self-elimination mechanism (Panel B). (Panel C) Proportion of transgene-free alleles (w, wild-type; v, self-elimination mechanism-generated resistant; u, no-cost resistant; r, high-cost resistant) after 60 generations under a range of self-elimination mechanisms (α) and self-elimination mechanism failure (γ) rates in the absence of natural resistance alleles (δ=0).

FIG. 26: Self-elimination may provide spatial control of gene drive transgenes at low, but not arbitrarily low thresholds. (Panel A) A single self-elimination mechanism failure through imperfect NHEJ-based repair at the nuclease recognition site. (Panel B) The inclusion of multiple nuclease recognition sites (red arrows, n=5) allows multiple independent attempts at self-elimination. Fitness parameters (Panel C) used in simulated release (Panel D) of gene drive-containing males at 1% of the population with 5 failures of the self-elimination mechanism required to create a self-elimination mechanism resistant allele (s); the formation of no-cost resistant alleles (u) was not allowed (δ=0). Model outcomes for four self-elimination mechanism rates (α=0, 0.1, 0.4, 0.8) are shown, all include a self-elimination mechanism failure rate of 1%. Arrow indicates a lag phase where gene drive frequencies approach, but can never reach, zero. (Panel E) If the proportion of gene drive alleles fell below the indicated threshold it was considered lost, and the maximum proportion of transgenic individuals (From T₀ to Tₗₒₛₜ or, if never reached, T₀ to T_{end}) was calculated. (Panel F) Potential spatial control provided by a self-elimination mechanism that was repressed conditionally during a contained field trial.

FIG. 27: Aedes aegypti transgenic strains for SSA-based transgene elimination. (Panel A) Schematic representation of the eukaryotic single-strand annealing (SSA) mechanism. The DNA double-strand breaks (DSBs), resulted by developmental processes or external damaging stimuli, can be repaired by SSA pathway in the presence of flanking direct repeat (DR) motifs. Following extensive DNA end resection from the DSB site by the MRN (MRE11-RAD50-NBS1)/CtIP complex, two DRs are aligned parallelly by RAD52 based upon sequence homology, and then the intervening sequence with a DNA damage is degraded (dotted lines). (Panel B) Schematic representation of plasmid constructs pBR-KmoEx4 and pSSA-KmoDR for the development of stage 1 kmo^{EGFP} and stage 2 kmo^{RG} strains, respectively. For pBR-KmoEx4, sgRNA-KmoEx4 was designed to target the exon4 of the Ae. aegypti kmo gene (Fig. S1A) and flanking kmo sequences (˜0.7 kb) were included as homology arms, HA1 (exon4/5) and HA2 (exon2/3). PUb-EGFP and RED_{1/2} (3′-half of DsRED) were interposed between the two HAs as transgene cargos. For pSSA-KmoDR, sgRNA-HybRED was created to target to RED_{1/2} in the kmo^{EGFP} strain (Fig. S1B). The stage 2 kmo^{RG} strain carries the additional kmo exon2/3 (HA2) as the DR sequences (pink bars) and 3×P3-driven full-sized DsRED, which was modified to contain the I-SceI recognition sequence next to ATG translation start codon. (Panel C) Transgenic mosquito larvae and adults expressing fluorescent markers. The kmo^{RG} strain had white-colored eyes due to the transgene-trapped kmo-null allele, DsRED fluorescent eyes due to the synthetic 3×P3 promoter activity, and the EGFP fluorescent body due to the ectopic polyubiquitin (PUb) promoter activity. The kmo^{EGFP} strain did not show DsRED fluorescent eyes (arrow heads), because it has RED_{1/2}, a truncated DsRED gene. (Panel D) PCR analysis for chromosomal integration of donor plasmid constructs at the kmo locus in the transgenic mosquitoes. Two pairs of PCR primers (horizontal arrows in FIG. 27; Panel B and Table 5) were utilized to recognize the junction areas between cargo genes and kmo genomic sequences outside of HAs.

FIG. 28: SSA-based transgene elimination was triggered by microinjection of a plasmid DNA expressing a homing endonuclease, I-SceI. (Panel A) Schematic workflow representation of evaluating the SSA-based transgene removal system engineered in the kmo^{RG} strain. The kmo^{RG} pre-blastoderm embryos were microinjected with a plasmid construct expressing the I-SceI enzyme, and the transiently expressed I-SceI induces DSBs at DsRED, a transgenic cargo gene. Theoretically, these DSBs are destined to go through three main repair paths, each of which can be developed as phenotypes of fluorescence markers and eye pigmentation in G₁ progenies. 1) If the I-SceI site would be intact due to no DSB or an error-free repair, the corresponding G₁ offspring would maintain the parental phenotypes, WGR (Kmo⁻, EGFP⁺, DsRED⁺). 2) If the DNA damage would be repaired by error-prone NHEJ, the coding frame shift could occur in DsRED, resulting in WG progenies (Kmo⁻, EGFP⁺, DsRED⁻). 3) If the DSB ends would be resected enough to activate SSA pathway, all transgenic cargos would be removed flawlessly and the wild-type kmo allele regained, and thereby the corresponding G₁ mosquito become the wild type displaying black-eyes (Kmo+, EGFP−, DsRED−). (Panel B) Distinct DNA repair-associated phenotypes in eye pigmentation and marker fluorescence of G₁ larvae in the SSA test. The insert is a magnified image of black-colored eyes restored by SSA-driven transgene elimination from the targeted kmo gene. (Panel C) Summary of the SSA test using a plasmid-based SSA trigger. The kmo^{RG} pre-blastoderm embryos, which were obtained from self-cross of heterozygous mosquitoes, were microinjected by pSLfa-PUb-I-SceI (0.5 μg/μl). EGFP-positive G₀ survivors (˜75%) were outcrossed with kmo^{Δ4} in a ♂:♀ ratio of 1:3, and G₁ larvae were screened for the DNA repair-associated phenotypes. W, Kmo⁻; Blk, Kmo⁺; G, EGFP⁺; R, DsRED⁺. The G₀ embryos without microinjection were analyzed for experimental controls.

FIG. 29: Transgenesis of kmo^{RG} mosquitoes was erased by an SSA trigger strain, Nos-I-SceI. (Panel A) Schematic representation of evaluating the SSA-based transgene elimination in kmo^{RG} by reciprocal crossing with Nos-I-SceI. F₁ offspring mosquitoes (SceI:kmo^{RG}) were outcrossed with kmo^{m} to determine DNA repair pathways selected for repairing I-SceI-induced DSBs. Depending on DSB repairs, their associated phenotypes are varied in F₂ mosquitoes; WGR (Kmo⁻, EGFP⁺, DsRED⁺) for No DSB, WG (Kmo⁻, EGFP⁺, DsRED⁻) for NHEJ, and Blk (Kmo⁺, EGFP⁻, DsRED⁻) for SSA. (Panel B) Summary of the single-generation SSA test using the Nos-I-SceI strain (G₁₂) as an SSA trigger. Following parental reciprocal crossing between Nos-I-SceI and kmo^{RG}, F₁ males and females (SceI:kmo^{RG}) were outcrossed with kmo^{Δ4} in a ♂:♀ ratio of 1:3, respectively. F₂ larvae were scored for marker fluorescence and eye pigmentation to measure the selection frequencies of a DSB repair pathway, either NHEJ % (WG/[WGR+WG+Blk]) or SSA % (Blk/[WGR+WG+Blk]). The screening results were separately collected, based upon the sex-dependent lineage of the SSA trigger allele, Nos-I-SceI. Experimental data were obtained from triplicated tests. Tukey's multiple comparison test (One-way ANOVA): P<0.0001.

FIG. 30: The nos-driven SSA is heritable to erase transgenesis from the cage-based population of the kmo^{RG} strain. (Panel A) DNA repair pathway-dependent phenotypes in the multi-generation SSA test (G₄). The F₁ mosquitoes (SceI:kmo^{RG}) from a parental cross (Table 9) of ♂ Nos-I-SceI×kmo^{RG} or ♀ PUb-I-SceI×kmo^{RG} were self-crossed. From F₂ screening, DSB repair-associated marker phenotypes (NHEJ % and SSA %) were scored from >1,000 pupae at every generation up to the F₆ generation. (Panel B) The SSA trigger-related phenotype throughout generations in the multi-generation SSA test (G₄). Frequencies of each transgene, Nos-I-SceI or PUb-I-SceI, were scored by the BFP⁺ percentages out of total larvae in every generation. (Panel C) DNA repair pathway-dependent phenotypes in the multi-generation SSA test (G₁₂). The F₁ mosquitoes (SceI:kmo^{RG}) from a parental cross (FIG. 3) of ♂ Nos-I-SceI×♀ kmo^{RG} or ♂ PUb-I-SceI×♀ kmo^{RG} were self-crossed in triplicate. From F₂ screening, DSB repair-associated marker phenotypes (NHEJ % and SSA %) were scored at every generation up to the F₅ generation. (Panel D) The SSA trigger-related phenotype throughout generations in the multi-generation SSA test (G₁₂). Frequencies of each transgene, Nos-I-SceI or PUb-I-SceI, were scored by the BFP⁺ percentages out of total larvae in every generation.

FIG. 31: The sgRNAs used for the development of kmo^{EGFP} and kmo^{RG} strains (SEQ ID NO: 32 and SEQ ID NO: 33). (Panel A) The sgRNA-KmoEx4 was designed to target the 4^{th} exon of the Ae. aegypti kmo gene locus, which is the landing site for HDR-mediated knock-in to generate kmo^{EGFP} strain (FIG. 27; Panel B). High Resolution Melting Analysis (HRMA) using a PCR primer pair of KmoEx4-F and KmoEx4-R (horizontal arrows) showed efficient activity of sgRNA-KmoEx4 to result in DSB-induced indel mutations in the Lvp wild-type mosquito genome. (Panel B) The sgRNA-HybRED was designed to recognize RED 1/2 in pBR-KmoEx4 created by blunted-end fusion of AscI and SbfI cuts. This allows for the HDR-mediated integration of the donor DNA, pSSA-KmoDR, to generate the kmo^{RG} strain (FIG. 27; Panel B). HRMA using a PCR primer pair of KmoEx4-F and DsRED-5Ra (horizontal arrows) showed efficient activity of sgRNA-HybRED to result in DSB-induced indel mutations in kmo^{EGFP} strain.

FIG. 32: Verification of the indel mutation resulted by microinjection of a plasmid expressing I-SceI to kmo^{RG} embryos. (Panel A) Schematic representation of the transgene structure in the kmo^{RG} strain. The I-SceI recognition site was engineered into the next to ATG translation start codon in the DsRED gene. Two direct repeat sequences (exon2/3, pink bars) were engineered flanking the transgene cargos. (Panel B) HRMA of the I-SceI site in DsRED for G₁ mosquitoes scored as WGR or WG. The PCR primer pair of DmHsp70-F and RED-5Ra (horizontal arrows, FIG. 32; Panel A) was utilized to amplify sequence variations at the I-SceI-induced DSB site. (Panel C) Sequencing analysis revealed a 4 bp deletion mutation in G₁ mosquitoes scored as WG (FIG. 32; Panel B) (SEQ ID NO: 35 shown in reference to SEQ ID NO:34). The ATG in bold letters is the translation start codon of DsRED gene and the I-SceI recognition site is underlined.

FIG. 33: Aedes aegypti transgenic mosquitoes as SSA triggers. (Panel A) Schematic representation of Mariner Mos1-based plasmid DNA constructs expressing I-SceI under the control of various promoters: nos and β2-tublin for female- and male-specific germline cells, respectively, and PUb and Hsp70A for ectopic and heat-inducible gene expression, respectively. Mos1 IRR, Mos1 inverse repeat right; Mos1 IRL, Mos1 inverse repeat left. (Panel B) SSA trigger strains expressing BFP marker in their eyes in both adults and 4^{th} instar larvae. (Panel C) RT-PCR analysis for I-SceI gene expression in SSA trigger strains. Total RNAs were purified from embryos at 24 hr post oviposition and utilized for cDNA synthesis. The primer pair of SceI-F and SceI-R was utilized to identify Nos or PUb-driven I-SceI transcripts, and the S7 primer pair for 40S ribosomal protein gene (RPS7) was used as the RNA control. The kmo^{m} strain was included as the control of no I-SceI transgene. For the experimental control, the same analysis was performed on the side in the absence of the reverse transcriptase (RT−).

FIG. 34: Verification of DSB repair-associated phenotypes resulted by reciprocal crosses between kmo^{RG} and the Nos-I-SceI strain. (Panel A) Schematic representation of the transgene structure in the kmo^{RG} strain. The I-SceI recognition site was engineered into the next to ATG translation start codon in the DsRED gene. Two direct repeat sequences (DR: exon2/3, pink bars) were engineered flanking the transgene cargos. (Panel B) HRMA utilizing the PCR primer pair of KMR1 and KMF2 (horizontal arrows, FIG. 34; Panel A) identified kmo^{Δ4} allele variations in F₂ mosquitoes with distinct phenotypes. W, white eyes; G, EGFP body; R, DsRED eyes; Blk, black eyes. (Panel C) HRMA utilizing the PCR primer pair of DmHsp70-F and RED-5Ra (horizontal arrows, FIG. 34; Panel A) identified sequence variations generated by I-SceI-induced DSBs in F₂ mosquitoes scored as WGR or WG.

FIG. 35: Sequencing analysis showing various indel mutations resulting from a I-SceI-induced DSB in F₂ mosquitoes scored as WG (FIG. 34; Panel C) (SEQ ID NOs: 36-64). The ATG in bold letters is the translation start codon of DsRED and the I-SceI recognition site is underlined. Red-colored letters indicate the newly inserted nucleotides and green-colored letters indicate nucleotide changes.

FIG. 36: The emergence of SSA-resistant alleles in a cage population of WGR mosquitoes during the multi-generation SSA test. (Panel A) Schematic representation of the transgene structure in the kmo^{RG} strain. The I-SceI recognition site was engineered into the next to ATG translation start codon in the DsRED gene. Two direct repeat sequences (DR: exon2/3, pink bars) were engineered flanking the transgene cargos. DmHsp70-F and RED-5Ra (horizontal arrows) are PCR primers to identify sequence variations generated by I-SceI-induced DSBs. (Panel B to Panel E) HRMA for I-SceI-induced indel mutations in mosquitoes scored as WGR in F₂ (Panel B), F₃ (Panel C), F₄ (Panel D) or F₅ (Panel E) generation. Delta (A) indicates nucleotide base deletion, and the plus mark (+) indicates the intact DsRED sequence.

### DETAILED DESCRIPTION OF THE INVENTION

The present invention provides technologies using vectors that can be pre-programmed to self-remove from eukaryotic genomes. In embodiments described herein, the programming can be based on the ‘lit-slow-fuse’ model, whereby no additional stimulus is required and the transgene slowly disappears over a number of generations, or on a ‘short-fuse’ model, whereby an external chemical-based trigger is applied (or removed) to rapidly trigger transgene self-removal.

The present invention provides a solution to a number of problems in the art regarding gene drive. For instance, the present invention provides a solution to a significant problem in the art regarding the seemingly counterintuitive goals of gene drive, namely, of both spreading a gene into a population to fixation (gene drive) and then completely removing the gene from the population (reversal). In further embodiments, the present invention provides solutions to the regulatory and political difficulties and hurdles associated with gene drive technologies.

The concept of driving genes into wild populations to control vector-borne diseases is known in the art. Genetic strategies to control dengue virus based on the release of sterile, transgenic mosquitoes have been successful where attempted. These types of strategies provide effective mosquito control only as long as releases continue, and thus represent a long-term financial and administrative commitment that must be maintained even in the absence of continued transmission. For this reason, gene drive systems that permanently convert the target population into a refractory state by spreading effector genes have been long sought after, as the release scale, duration, and costs associated with such systems are expected to be dramatically lower.

Engineering or harnessing chromosomal translocations, meiotic drive systems, transposable elements, maternal-effect dominant embryonic arrest, engineered under-dominance, and homing endonuclease genes (“HEGs”) to achieve the goals of a gene drive-based vector control campaign have been slowed or prevented by the technical challenges associated with these systems. The rapid development of clustered regulatory interspaced palindromic repeat (“CRISPR”) editing reagents introduced a new programmable nuclease that did not suffer from the problems of HEGs (which are difficult to engineer) or transcription activator-like effector nucleases (“TALENs”) (which are poor repair substrates). The advent of site-specific gene editing using CRISPR/Cas9 reagents has produced a wave of successful gene drive experiments in yeast, flies, and mosquitoes.

With the ease of developing new Cas9-guided nucleases, the concept of gene drive is now spreading out to potentially control invasive or unwanted species, as well as other applications. However, there is at present no solution that addresses how one could achieve two seemingly counterintuitive goals of both spreading a gene into a population to fixation of a problem and then completely removing the gene from the population. The ease with which CRISPR nucleases can be generated, combined with the highly effective nature of CRISPR-based gene drive in Drosophila, has led to calls for increased regulatory capacity and institutional oversight of CRISPR-based gene drive approaches, with some even calling for the prohibition of public discussion of the details due to fears of bioterrorism.

Many of the proposed concepts and suggestions in the art to control or “reverse” the drive and transgenes introduced thereby are inadequate, as they would require coordinated additional large scale field releases of secondary or tertiary transgenic strains to fight against a first failed strain. The drawbacks are many. On the technical side, one would not know if the secondary strain will work until a field release occurs. From a regulatory perspective, each strain would likely be evaluated and approved (or not) on its own merits, and there is no precedent for a conditional deregulation for release of a product X contingent upon a product Y. Finally, from a political viewpoint, local authorities may choose to end a trial abruptly for any number of reasons, many of which may have nothing to do with the technical details. In such cases, those participating in the releases may simply not have the opportunity to release additional remediating strains.

To address the difficulties and shortcomings of the prior art, strategies have been crafted, as discussed herein. The present application describes gene drive-based technologies using vectors that are pre-programmed to self-terminate. In certain embodiments described herein, the programming can be based on the “Lit Slow Fuse Model,” whereby no additional stimulus is required and the transgene slowly disappears over a number of generations, or on a “Short Fuse Model,” whereby an external chemical-based trigger is applied (or removed) to rapidly trigger transgene self-removal.

Several independent mechanisms for use in the self-elimination, gene drive-based technologies of the present invention are also described in the present application. In certain embodiments, these mechanisms include a recombinase-based mechanism, a transposase-based mechanism, and a single-strand annealing (SSA)-based mechanism. The self-elimination systems of the present invention may be incorporated into a gene drive approaches to limit the transgene persistence in nature.

Such a biodegradable system would allow for extensive field-based trials of functional gene drive systems (or any transgene), while essentially setting a time limit on the presence of the transgene in nature. This would allow accurate and meaningful assessments of both risks and benefits of the technology, including effects on the target population (such as size, density, behavior, ability to transmit non-target pathogens), as well as any changes in the surrounding ecosystem or effects on human health.

### Gene Drive

In the context of the present application, “gene drive” refers to any mechanism that results in the inheritance of a gene at a probability greater than would be expected by strict Mendelian inheritance.

Compositions and methods are known in the art regarding programmable nucleases that can introduce a double stranded break (“DSB”) at predetermined locations in a genome to facilitate gene drive. Such breaks are then repaired using the homologous chromosome as a template, a process termed homology-dependent repair (“HDR”), resulting in duplication of the transgene into the repaired chromosome. However, other cellular repair pathways such as non-homologous end-joining (“NHEJ”) and single-strand annealing (“SSA”) compete for access to the DSB. Repair through these pathways does not result in duplication of the transgene into the repaired chromosome and thus does not lead to gene drive. In certain embodiments, the present invention takes advantage of these alternate repair pathways to halt gene drive and promote self-elimination of the inserted transgenes.

The various models, as well as embodiments demonstrating application of these models, are described below.

### A Lit Slow Fuse Model

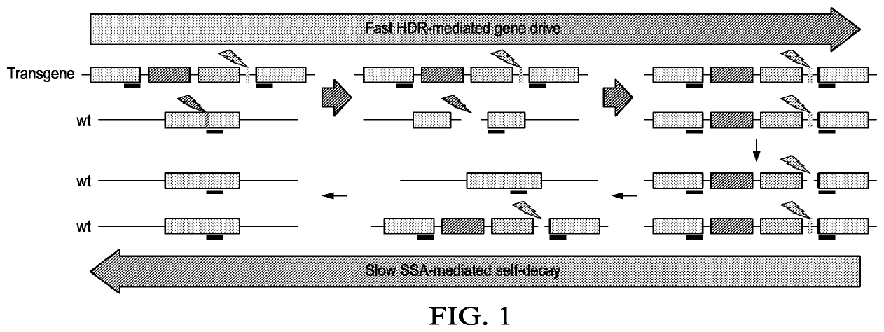

In certain embodiments, the present invention provides a self-elimination model referred to as “The Lit Slow Fuse Model.” This model enables removal of transgenic sequence from the target population without intervention. As shown in FIG. 1, under the Lit Slow Fuse Model, in one embodiment, a transgene includes two site-specific nucleases (as shown on the transgene in the upper left) expressed in unequal quantities. DNA break induction by the first nuclease on the opposite chromosome, followed by homology-based repair increases the transgene copy number and results in gene drive through methods understood in the art. Lower expression of the second nuclease results in low level DNA break induction specifically in the inserted transgene. The expression of each nuclease can be controlled by distinct regulatory elements (promoters), through the use of an IRES (Internal Ribosome Entry Site), the use of alternative splice acceptors, viral peptides, self-splicing inteins, or any other such method known to the art. Repair of the breaks within the inserted transgene via the SSA pathway results in complete loss of all transgene sequence. The black bars shown in FIG. 1 indicate tandem duplicated sequences that drive SSA-based repair. In particular embodiments, as part of the construct containing the inserted transgenes, nucleases, and duplicated sequences driving the SSA-based repair, a single nucleotide polymorphism is also included. Due to the nature of the subsequent SSA-based repair, this polymorphism is included in the repaired chromosome, resulting in a sequence that no longer contains the exact sequence recognized by the first nuclease and thus are no longer susceptible to the first nuclease, preventing re-invasion.

### A Short Fuse Model

In other embodiments, the present invention provides a self-elimination model referred to as “The Short Fuse Model.” This model enables removal of all transgenic sequence from the target population, but will require intervention such as the addition or removal of a chemical trigger. When the trigger is added or removed, such action will rapidly trigger self-removal of the transgenic sequence.

In certain embodiments, the Short Fuse Model involves conditional expression systems, such as those based on the bacterial tetO operon, which is efficiently repressed in the presence of tetracyline, and the yeast GAL4-UAS system. Such conditional expression systems allows for controlled activation of the nuclease resulting in the self-elimination of the transgene. This in turn provides conditional or controlled transgene self-elimination.

Many conditional expression systems are known in the art and are useful in present invention. These include the bacterial tetO operon, the yeast GAL4 system, the Neurospora Q system, simple heat-shock or metal-induced gene expression systems, GeneSwitch, and so forth.

### Vector Constructs

It is understood in the art that SSA-based DNA repair is triggered by direct repeats flanking a DNA break, and is influenced by the length of direct repeats, as well as their spacing. To effectively harness this pathway in order to pre-program the elimination of transgene sequences from an insect population, embodiments of the present invention involve the generation and insertion into the genome of certain organisms a synthetic construct containing, for instance, a reporter, a nuclease expressed in the germline, and a corresponding unique target site.

Reporters that may be employed in various embodiments described herein are known to those of ordinary skill in the art. Reporters generally expected to achieve the desired results as described herein include fluorescent proteins such as EGFP and DsRED, as well as physical mutations in the target organisms influencing pigmentation/coloration.

Nucleases that may be employed in various embodiments described herein are known to those of ordinary skill in the art. Nucleases generally expected to achieve the desired results as described herein include those based on CRISPR/Cas9 or related CRISPR nucleases, as well as homing endonucleases such as I-Sce (yeast), I-Cre (Chlamydomonas reinhardii), and I-Ani (Aspergillus nidulans).

Various unique target sites can be selected for use in the embodiments described herein. In general, the target sites described herein for the self-eliminating transgene are not found in the genome of the host organism. Once introduced into the organism's genome, they would be a unique target for the nuclease. Target sites described herein are capable of being cleaved by a nuclease, as described herein. In some embodiments, the target site includes a random synthetic string of 20-24 nucleotides.

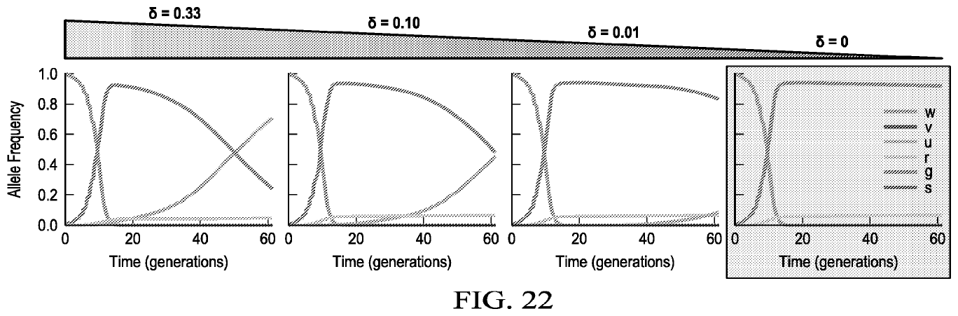

In certain embodiments, a vector construct of the present invention may contain, for instance, a desired gene drive transgene, any desired reporters or markers, and any desired cargo genes accompanied by a gene encoding a recombinase, wherein the entire cassette may be flanked with corresponding recombination sites. In such an embodiment, expression of the recombinase would result in intramolecular recombination between the two flanking regions resulting in the excision of the intervening gene drive transgene, as well as all other transgenes, and restoration of the host allele (FIG. 18A).

In another embodiment, a vector construct of the present invention may contain, for instance, a desired gene drive transgene and other transgenes, including, but not limited to any desired reporter, marker, and/or cargo genes, accompanied by a gene cassette encoding an integration-deficient transposase, and flanked with corresponding inverted terminal repeats (ITRs, FIG. 18B). In such an embodiment, expression of the transposase would result in binding of the transposase to the ITRs and initiation of targeted double-stranded DNA breaks, resulting in the loss of all transgene sequences. The subsequent repair of the gap would result in the restoration of the host allele.

In yet another embodiment, a vector construct of the present invention may contain, for instance, a desired gene drive transgene and other transgenes, including, but not limited to any desired reporter, marker, and/or cargo genes, flanked by a direct repeat corresponding to the wild type host allele. In this embodiment, all transgene sequences are susceptible to loss via SSA-based DNA break repair (SSA, FIG. 18C). Homology between the two repeated sequences may promote SSA-based repair following a double-stranded break, resulting in the loss of all transgene sequences and restoration of the host allele (FIG. 18C). In certain embodiments, a site-specific nuclease can be directed to generate a targeted DNA break, not in the host gene, but in the transgenic construct itself. Such a second nuclease could, in one embodiment be independently coded from the transgenes involved in the gene drive. In another embodiment, a DNA break could simply be generated from the inclusion of an independent synthetic guide RNA, different from that used for the gene drive.

The above embodiments result in in cis removal of all transgene sequences while simultaneously generating a transgene-free allele that is resistant to future cleavage by the same gene drive mechanism. While the use of a recombinase would leave behind a scar that might perturb the activity of the host gene, silent nucleotide changes may be incorporated into either the transposon- or SSA-based approaches to preserve the wild-type amino acid sequence at the target gene and still provide resistance to further cleavage by the gene drive mechanism.

Various embodiments described herein will further include a control sequence to initiate transgene elimination, as in some embodiments, the engineered nuclease is to be switched on in the developing germ cells. To accomplish this requires control sequences capable of regulating gene expression in this manner. Many such control elements have been functionally characterized for both Drosophila and Ae. aegypti and are known in the art. For example, the female germline specific promoter nanos has been used in Drosophila to efficiently drive the expression of ΦC31 integrase and now Cas9 in the female germline.

The Ae. aegypti nanos promoter has previously been shown to successfully drive transposase expression in the female ovaries. Other germline promoters are also known in the art. For instance, germline promoters have been validated in mosquitoes and include for example vasa, VgR, β-tub. Additionally, genes such as β-tubulin are conserved between Drosophila and Tribolium. In some embodiments, RNA-seq approaches can reveal other germline-specific gene candidates.

In other embodiments, in addition to directly controlling nuclease expression, conditional expression systems, such as those based on the bacterial tetO operon may be employed, which is efficiently repressed in the presence of tetracyline, and the yeast GAL4-UAS system. Thus, nuclease activity, and in turn transgene self-elimination, can be controlled by the experimenter.

The organism selected for use in various embodiments described herein can be any eukaryote. In some embodiments, the organism is an insect species such as Drosophila melanogaster, Aedes aegypti, and Tribolium castaneum, a plant species, or an animal. In further embodiments, the organism is a human. A person having ordinary skill in the art will understand that certain parameters may be adjusted for individual species, such as hormone concentrations, culture conditions, strains of Agrobacterium, and incubation periods.

Embodiments described herein may depend on the recognition of the direct repeats engineered to flank the synthetic construct by the cellular SSA-repair machinery prior to initiation of repair by the end-joining machinery, a result that would remove the nuclease target site (preventing any further cutting) while leaving the synthetic construct intact. It is anticipated that each organism may display different preferences for default DNA repair (as the genome architecture of each varies), and the optimal size and spacing of direct repeats, as well as their distance from the nuclease cleavage site may vary in each organism. It is expected that a common set of rules may be established regarding size and spacing of direct repeats for related genomes. Increasing the length of the direct repeats, decreasing the spacing between repeats, and decreasing the distance between the nuclease cleavage site and one of the repeats are all expected to shift the balance to some extent towards SSA-based repair and away from end-joining. Increasing the number of nuclease sites may also help to overcome low-level end-joining repair. Strategies that directly interfere with end-joining repair globally may be avoided in certain embodiments, as this may result in unknown changes elsewhere in the genome.

In certain embodiments, such as those involving A. palmeri, in order for gene-deletion to work in, the synthetic repeat regions within the introduced construct need to be recognized by the endogenous homologous recombination (“HR”)-based repair pathway with priority over the NHEJ pathway. Preference for these mutually exclusive pathways differs in each organism, and such preference should be assessed for each organism.

### Applications for the Models

Various embodiments described herein may be useful for efforts to use genetics-based strategies to control transgenic sequences in any organisms. The strategies described herein may be used in the field of agriculture, synthetic biology, and even human medicine. For instance, mosquito-borne diseases such as dengue, malaria, chikungunya, Zika, and so forth, may be controlled or addressed using the described gene drive-based strategies. An organization testing an experimental gene drive strategy to fight malaria may wish to pre-program the elimination of the transgene from any mosquito that escapes from the study. The embodiments described herein may also be useful to eliminate transgenes in seeds so that seeds are not used in an unauthorized or undesired manner. Additionally, the embodiments described herein may be used in human gene therapies in a manner so as to allow for triggering the removal of a transgene in the event of an adverse reaction in a patient. A person of skill in the art will understand the numerous applications that can employ embodiments described herein.

### EXAMPLES

The following examples provide illustrative embodiments of the invention. However, those of skill in the art should, in light of the present disclosure, appreciate that many changes can be made in specific aspects of these embodiments without departing from the concept, spirit, and scope of the invention. Moreover, it is apparent that certain agents which are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### Validation of a Self-Eliminating Transgene in Drosophila

### Example 1: Assembly of Donor Constructs with Direct Repeats Flanking

Site-specific gene insertion will be used to generate several cohorts of transgenic Drosophila. Each transgenic strain will contain a set of direct repeats flanking a visible fluorescent marker and a nuclease gene programmed to recognize the introduced transgene. Nuclease cutting followed by SSA-based repair using the engineered direct repeats is intended to eliminate all transgene sequences, resulting in restoration of body pigmentation. As flies have a shorter generation time than mosquitoes and are easier to rear, it is anticipated that the assessment of variant constructs and loss in the context of an active Cas9-based gene drive will occur fairly rapidly.

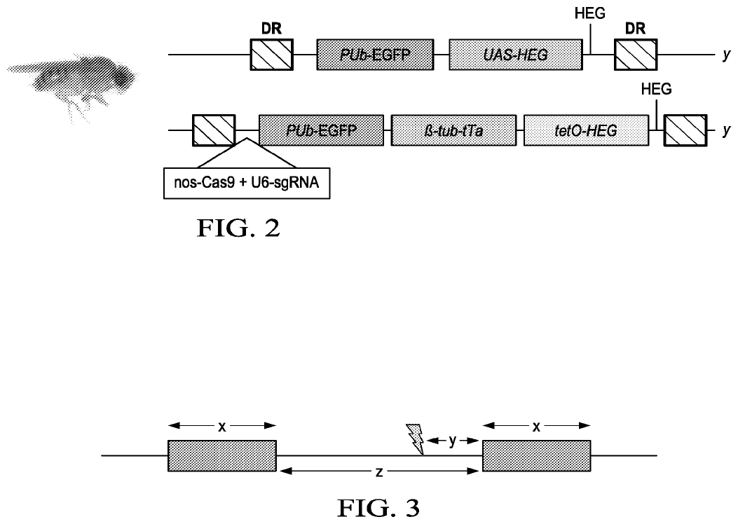

FIG. 2 shows the UAS-driven or tetOff-controlled nuclease expression constructs that will be used to trigger transgene self-elimination in this series of experiments.

Table 1 below lists the donor constructs for the generation of fly strains containing self-eliminating gene cassettes.

TABLE 1
Donor constructs.

| | Direct repeat | Control of | Active Cas9 |
|---|---|---|---|
| Donor Construct | length | nuclease | present? |
| 1 | 1000 bp | UAS/Gal4 | N |
| 2 | 1000 bp | UAS/Gal4 | Y |
| 3 | 1000 bp | Tet-Off | N |
| 4 | 1000 bp | Tet-Off | Y |
| 5 | 2000 bp | UAS/Gal4 | N |
| 6 | 2000 bp | UAS/Gal4 | Y |
| 7 | 2000 bp | Tet-Off | N |
| 8 | 2000 bp | Tet-Off | Y |
| 9 | 4000 bp | UAS/Gal4 | N |
| 10 | 4000 bp | UAS/Gal4 | Y |
| 11 | 4000 bp | Tet-Off | N |
| 12 | 4000 bp | Tet-Off | Y |

Constructs generated in Example 1 will be employed in Example 2.

### Example 2: Generation of Transgenic Fly Strains with Confirmed Nuclease Expression

Each plasmid construct generated in Example 1 will be injected into Drosophila embryos using standard techniques along with a synthetic guide RNA targeting the yellow gene and Cas9 mRNA. After crossing the surviving individuals, transgenic progeny will be identified by the expression of the fluorescent reporter. Such individuals will also present a loss of body pigmentation due to disruption of the yellow gene when made homozygous. The landing site of each integration event will be confirmed through PCR of genomic DNA. Only those strains bearing a transgene insertion into the yellow gene with all components intact will be retained. Pre-existing Gal4-driver lines will be obtained from stock centers to activate germline-specific expression of the nuclease for those strains under the control of the UAS. As the landing site is held constant, only a single verified homozygous strain for each construct will be employed in Example 3.

### Example 3: Evaluation of Transgene Loss and Phenotype Reversion at the Individual Level

For each fly strain, the percentage of progeny that contain or have lost the inserted transgene will be determined. For UAS-nuclease strains, each strain will be crossed with an appropriate Gal4-driver (nos-Gal4, vasa-Gal4 or similar) to yield expression of the nuclease in the fly germline. For strains with nuclease under the control of the tet-Off system, flies will be reared in the absence of tetracycline. In both scenarios, nuclease cutting of the transgenic construct followed by SSA-based repair will result in the loss of the fluorescent reporter and simultaneous restoration of body pigmentation. As only homozygous flies will be used for these experiments, the presence of the Cas9 transgene cannot result in any further drive, but effectively increases the length of the transgene sequence.

By varying the length of the direct repeats, the influence of this parameter on the successful use of SSA-based repair and transgene elimination will be determined. By varying the Gal4-driver line used (or the amount of tetracycline used), data is expected to yield information concerning the relationship between the strength of nuclease expression and the efficiency of transgene elimination.

### Example 4: Evaluation of Transgene Loss and Phenotype Reversion at the Cage Population Level

The ability of self-eliminating transgenes to remove themselves from a laboratory cage population in the presence of an active Cas9-based gene drive system will be assessed. Fly strains containing active Cas9 (based on constructs 2, 4, 6, 8, 10, 12) will be introduced at various frequencies (1%, 10%, 25%, 50%) into wild-type cages. For UAS-nuclease strains, cage populations will be fixed for a particular Gal4-driver. For tet-Off strains, flies will be reared on various levels of tetracycline (based on observations from Example 3). For each generation, the percentage of transgenic flies (fluorescent reporter, yellow) will be determined, with flies propagated blindly for 10 generations.

The output for this Example will be data concerning the rate of transgene loss that is expected to overcome the driving ability of site-specific nucleases. This is expected to provide a paradigm-shifting safety feature for working with driving transgenes in situations (such as field releases) where they otherwise could not be controlled or removed.

### Optimizing Parameters for SSA-Based Programmed Transgene Elimination in Ae. aegypti

Intrachromosomal deletions mediated by the SSA pathway can result in deletions of at least 80 kbp at high efficiency with the length of repeats and distance of separation strongly influencing this mode of repair in yeast, flies, and vertebrate cells. The length of sequence that can be effectively collapsed in turn dictates the length of a minimal nuclease-based gene drive system (along with any visual markers and anti-pathogen gene cassettes). It is known that critical factors for NHEJ, HDR, and SSA are conserved in mosquitoes, where complete deletion of more than 2 kb using very short (200 bp) repeats have been observed.

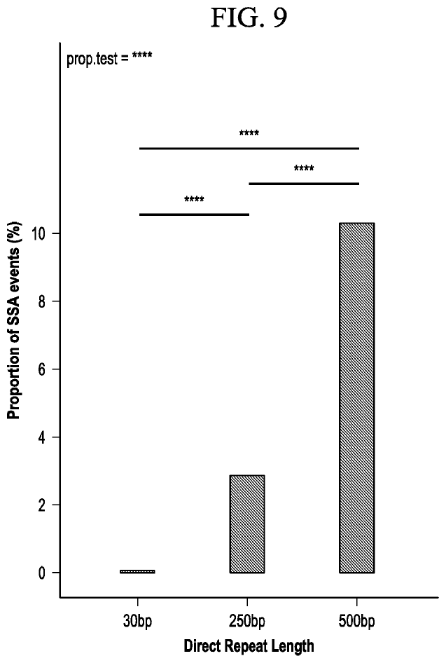

In prior studies, three different HEGs have been used to introduce double-stranded DNA breaks in the Ae. aegypti germline. Using two transgenic strains where the EGFP fluorescent marker was flanked by HEG recognition sites, progeny have been recovered that had lost EGFP expression following injection of HEG expression constructs into pre-blastoderm embryos. Two types of repair events were recovered: NHEJ following cutting at each HE recognition site flanking the EGFP gene (Y2-I-Anil only) and SSA-based repair following cutting a one HE site (I-SceI, I-CreI, Y2-I-Anil) (as shown in FIGS. 6A and 6B). Nucleases with greater activity are associated with both NHEJ and SSA, while those with a reduced activity appear to be exclusively repaired using SSA.

Other studies have shown repeats as short as 34 bp were able to direct the collapse of ˜1500 bp of intervening sequence, but not ˜2700 bp. A longer repeat length of 195 bp enabled the collapse of ˜2400 bp of transgenic sequence. These data suggest that extending the repeat length in Ae. aegypti will increase the efficiency of performing SSA-based repair over longer intervening sequences. It should be noted that, in this study, the site of DNA break induction was between 50-150 bp from the start of one (or both) of the repeats. Reported rates show targeted transgene integration into multiple loci in the Ae. aegypti genome using CRISPR/Cas9 at rates similar to traditional transposon-based methods.

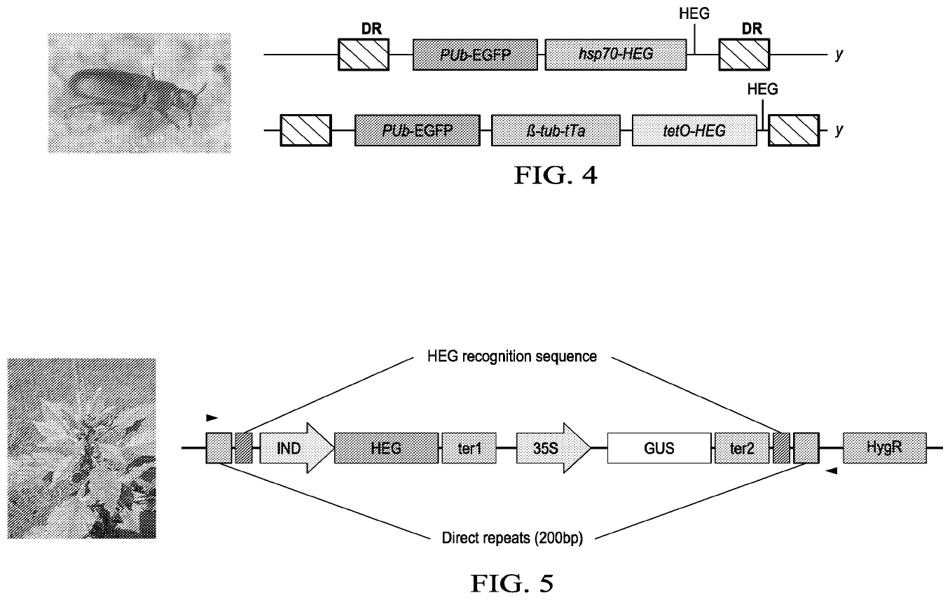

However, the SSA pathway has never been explored in any mosquito species. Thus, an analysis of each of these parameters on SSA-based repair in the Ae. aegypti germline is essential to realizing the full potential to engineer self-decaying gene drive systems for this organism. FIG. 3 shows a diagram of the parameters to analyze that may affect the rate of SSA-based repair of dsDNA breaks (self-decay). The length of the direct repeats (X), distance between repeat and DNA break (Y) and distance between repeats (Z) are expected to contribute to SSA efficiency. A series of experiments have been designed to assess and optimize these parameters.

### Example 5: Establish Transgenic Ae. aegypti Bearing the Pre-Programmed Self-Excising Cassette in the Genome

CRISPR/Cas9 will be used to stimulate dsDNA break induction adjacent to an existing PUb-EGFP transgene previously inserted into the kmo locus. Homology-dependent repair will be used to incorporate one of three variant transgene cassettes (varying only in the length of the direct repeat: 1000 bp, 2000 bp, 5000 bp), with each marked with DsRED. Each cassette will also include a germline-specific promoter (nanos, vasa or β-tubulin; all of which have been characterized in Ae. aegypti) driving the expression of the tTa transactivator; a homing endonuclease under the control of the tetO promoter, and the corresponding homing endonuclease target site. The integration of each cassette will be confirmed by the stable inheritance of DsRed in subsequent generations and through PCR/Southern analysis of the integrated transgene.

This Example 5 will yield three transgenic strains of Ae. aegypti, with each marked with a specific phenotype: white-eye (visible), red eye (fluorescent), green body (fluorescent).

### Example 6: Determination of the Effect of Repeat Length on Transgene Self-Elimination in Ae. aegypti

Once established, embryos from each of the three lines generated in Example 5 will be collected from females reared in the absence of tetracycline. This releases the tTa from repression and activates expression of the homing endonuclease which is expected to induce specific DSBs in the engineered transgene. While end-joining repair can result in the loss of DsRED fluorescence through disruption of the ORF, restoration of eye pigmentation can only occur following SSA-based collapse of the direct repeats. Thus, the rate of both SSA-based repair (Black eye, EGFP⁻,DsRED⁻) and NHEJ-based repair (White eye, EGFP⁺, DsRED⁻) can be determined by scoring progeny. It is expected that as the repeat length increases, so will the number of SSA-based events; inversely, NHEJ events are expected to decrease. Repair events will be confirmed through molecular analysis.

Example 6 will provide experimental verification of the role of repeat length in influencing SSA-based repair efficiency in Ae. aegypti. The most efficient construct will be chosen for use in Example 7.

### Example 7: Determination of the Effect of Distance Between the DSB Site and the Direct Repeats on Transgene Self-Elimination

Using the most efficient strain from Example 6, either HDR or recombination-mediated cassette exchange will be used to generate two additional variant strains, essentially replacing the initial HEG to one of two alternative HEGs with an independent target site located at varying distances from the direct repeats. Once established, mosquitoes bearing each of the three HEGs will be reared in the absence of tetracycline to activate HEG expression and initiate targeting at distances of ˜300 bp, 1000 bp, and 4000 bp from the direct repeats. Once again, successful SSA-based collapse will eliminate both fluorescent markers and restore eye pigmentation. For the HE site at the start of the EGFP ORF, NHEJ-based repair can result in loss of EGFP fluorescence, permitting tracking of both repair types. It is expected that as the distance between the dsDNA break site and repeats increases, the number of SSA-based events will decrease, while NHEJ events will increase.

Example 7 will provide experimental data concerning the practical effect of distance between nuclease cut site and direct repeats on the efficiency of pre-programmed transgenes to undergo self-elimination.

### Example 8: Determination of the Effect of Distance Between Repeats (Cargo Size) on SSA-Based Repair

One of the three transgenic strains generated in Example 7 will be selected and the ΦC31 integrase system will be used to incorporate one of two additional transgenes into the existing locus via attP:attB recombination. Homology-dependent integration cannot be used in this case, as SSA would compete for the free DNA ends. Each ΦC31-integrated transgene will be marked with a blue fluorescent protein (mTagBFP). The ΦC31-integrated transgenes will increase the spacing between the direct repeats from 4 kbp to 8 or 16 kbp; integrations will be confirmed by PCR on genomic DNA over the resulting attL and attR flanking regions. Once established, mosquitoes bearing each transgene will be reared in the absence of tetracycline to activate HEG expression and progeny scored for eye color, as well as EGFP, mTagBFP and DsRED fluorescence. Once again, successful SSA-based repair will eliminate all fluorescent markers and restore eye pigmentation. NHEJ-based repair will be tracked through the loss of DsRED (or EGFP, if an alternative HE site is used), again permitting tracking of both repair types. It is expected that increasing the distance between direct repeats may decrease the number of SSA-based events and increase NHEJ events, but it is highly possible that even at the distances used SSA-repair could remain extremely efficient.

Experimental data concerning the practical effect of distance between the direct repeats on the efficiency of pre-programmed transgenes to undergo self-elimination will be generated.

### Self-Eliminating Transgenes Control CRISPR-Based Gene Drive in the Mosquito Ae. aegypti

Germline promoters will be evaluated for their ability to produce functional Cas9 protein and initiate gene drive in Ae. aegypti mosquitoes. The best candidate (best homing rate, least effect on fitness) will be chosen for incorporation into the self-eliminating transgene locus developed in Example 5. In the presence of tetracycline, the HEG is repressed and Cas9-based gene drive through a laboratory population is expected to proceed rapidly. In the absence of tetracycline, activation of the HEG triggers DSB induction at the transgene and if followed by SSA-based repair is hypothesized to eliminate all transgene sequences, resulting in restoration of eye pigmentation and complete loss of the gene drive system.

### Example 9: Generation of Transgenic Mosquito Strains to Evaluate Various Promoters for their Ability to Produce Functional Cas9 in the Ae. aegypti Germline

While efficient gene drive constructs have been reported in Drosophila and Anopheles mosquitoes, this technology has not been developed for the Ae. aegypti, the primary vector of dengue, chikungunya and Zika viruses. Transgenic strains will be generated carrying an active Cas9 and sgRNA targeted to the kmo gene. The use of the kmo gene as a landing site simplifies the detection of homozygotes, as these individuals will be white-eyed. The completed construct from Example 5 will be modified to contain a nos-Cas9, vasa-Cas9 or β-tub-Cas9 cassette as well as a U6-sgRNA cassette. The resulting plasmid will be injected into pre-blastoderm Ae. aegypti embryos (kmoEGFP strain). DsRed+EGFP+ progeny containing the full set of transgenes will be crossed by the parental strain to establish the line, which will be referred to as kmosd (self-decay). A combination of Southern analysis and genomic DNA PCR/sequencing will be used to verify the landing site of the donor construct and the integrity of the components.

This experiment will generate three transgenic strains that vary only in the promoter controlling germline-specific Cas9 expression.

### Example 10: Evaluation of the Baseline Rate of Gene Drive in kmosd Ae. aegypti

For this experiment, the nuclease controlling transgene self-elimination will be repressed by tetracycline so that an assay on the performance of the gene drive components may be completed. For each strain containing active Cas9 from Example 9, kmosd male mosquitoes will be mated with wild-type females and all progeny scored for fluorescent markers and eye pigmentation. Similar experiments will be performed by mating kmosd females with wild-type males to assess the effect of maternal versus paternal gene drive. Those strains displaying the expected super-inheritance of the transgene will be assessed for fitness costs. kmosd individuals will be assessed for effects on longevity, ability to procure a bloodmeal, time to oogenesis, and number of viable progeny produced. The strain displaying the best compromise of successful gene drive and lowest fitness cost will be introduced at various frequencies (1%, 10%, 25%, 50%) along with the corresponding number of wild-type individuals into large cages with a cohort of the opposite gender for large-scale laboratory cage trials. For each generation, the percentage of kmosd mosquitoes (DsRED+, EGFP+, white eye) will be determined, with mosquitoes propagated blindly for 5 generations.

An optimal control sequence will be selected for performing Cas9-based gene drive in the Ae. aegypti germline, and establish initial parameters for both the effectiveness of this drive and its effect on mosquito fitness.

### Example 11: Evaluation of the Baseline Rate of Gene Decay in Homozygous kmo^{sd} Mosquitoes

Mosquitoes carrying two copies of the kmo^{sd} allele will be generated through standard crossing and reared in the absence of tetracycline to activate the HEG and stimulate transgene self-elimination. After mating with homozygous kmo^{sd} males, kmo^{sd} females will be offered a bloodmeal; 50-100 fully fed females will be transferred individually into single tubes for egg collection. For each female, progeny will be screened for the black eye phenotype, as well as EGFP and DsRED markers, both of which would be lost upon SSA-mediated repair. At least three replicate experiments will be performed per generation, with a target of at least three generations. The percentage of black-eyed individuals divided by the total number screened will be calculated (the rate of decay) for each female. To confirm that identified black-eyed mosquitoes are indeed the result of SSA-mediated self-decay, and not simply contaminants from a wild-type genotype, HRMA and/or sequencing on PCR amplicons derived from where the duplicated region was collapsed in each individual will be performed. SSA-mediated collapse will result in an in-frame silent base substitution that was built into the donor construct, enabling differentiation from wild-type.

It is expected that empirical data will be generated on the rate of pre-programmed transgene self-elimination in the mosquito germline after fixation of a Cas9-containing gene drive Ae. aegypti, both in the context of a single generation and through the analysis of a multi-generational large cage population.

### Self-Eliminating Transgenes from an Agricultural Pest

Described herein are experiments that will rely on the ability to perform site-specific gene integration into each described target species. This technology described has commonly been used for the model organism Drosophila, and has been successful with mosquito, as well. Strategies similar to those known in the art for performing perform site-specific gene integration into model organisms such as Drosophila and the mosquito will be used to perform such manipulations in other organisms, including beetles and plants. The appropriate length/spacing of the direct repeats needed for single-strand annealing-based DNA repair, and determining the optimal expression level of the controlling nuclease to achieve transgene self-elimination on the desired timescale will be determined for each organism tested. Various synthetic biology approaches will be used to vary both parameters extensively in a number of target organisms.

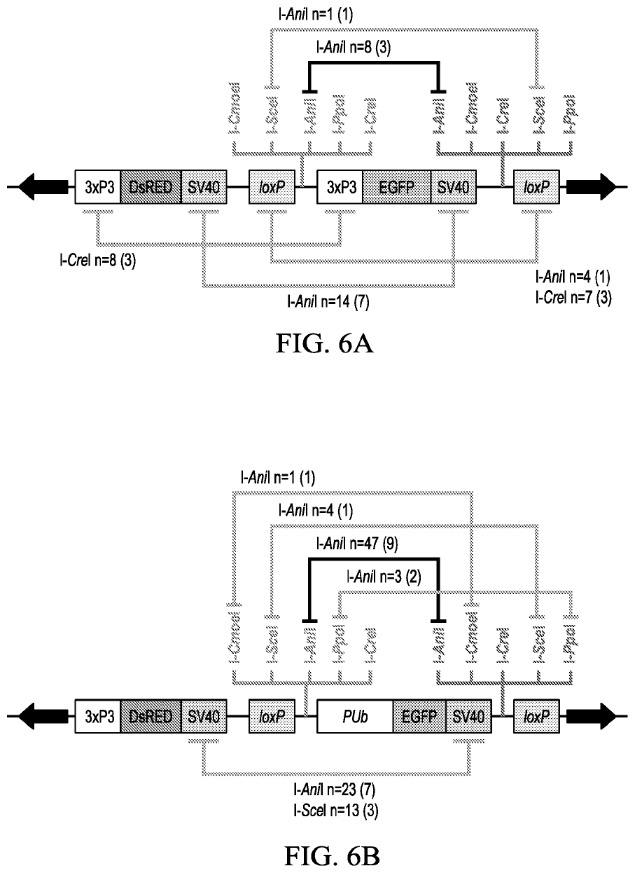

Tribolium castaneum is a model insect species and a pest of stored grain. Transformation of this insect is routine, and both it and many of its coleopteran relatives are major pests of agriculture. Transgenic T. castaneum will be generated with the self-eliminating transgene construct and demonstrate use of the system in pests of agriculture. The experimental approach and timeline will be similar to that described for flies and mosquitoes. FIG. 4 shows the self-eliminating transgene in Tribolium. Shown are direct repeats (“DR”) flanking a fluorescent marker (EGFP) following by either a heat-inducible promoter (hsp70) or the bipartite tetO system to control nuclease expression. Nuclease activation (HEG) triggers DNA break induction and repair using SSA to eliminate all transgenic sequences.

### Example 12: Validation of Germline-Specific Promoters in Transgenic Tribolium

Transgenic technology is well established for Tribolium, and despite the availability of a number of characterized promoters, no work has yet been published concerning germline-specific promoters in this organism. The male germline-specific gene β-tubulin has been identified in Tribolium and is a clear ortholog of the Drosophila gene; β-tubulin promoters have been extensively characterized and used for driving transgene expression in flies and mosquitoes. To verify that the Tribolium β-tub is expressed in a similar manner, and to potentially identify other candidate promoters capable of driving transgene expression in the germline, RNAseq will be performed on dissected ovaries, testes, newly deposited eggs and carcasses. Genes highly expressed in male/female gametes and/or early embryos compared to adult carcasses will serve as sources of new control elements. Each putative control element will be placed upstream of a fluorescent reporter and transgenic beetles will be generated, which is a highly efficient process in this insect. Three transgenic strains will be evaluated for EGFP expression for each candidate promoter.

The germline-specific expression of the Tribolium β-tub promoter will be confirmed, and at least three other candidate genes possessing germline-specific expression will be identified.

### Example 13: Generation of Transgenic Tribolium Containing the Self-Eliminating Transgene Cassette

Easily scored eye-color mutants (vermillion) for Tribolium are available and have been extensively characterized. Also identified is a clear ortholog of the Drosophila yellow gene that controls body pigmentation. Both of these genes may be used as landing sites for the site-specific integration of the self-eliminating transgene cassette, as described for flies and mosquitoes. CRISPR-Cas9-based gene editing will be employed to introduce a double-stranded DNA break in either vermillion or yellow to allow incorporation of each of the constructs listed shown in FIG. 4. Molecular analyses will be used to confirm the integrity of the transgene and the landing site. The use of a heat-inducible promoter (hsp70) allows an alternative to control the activity of the nuclease in addition to a germline (β-tub or other) promoter controlling the bipartite tet-Off system. Two transgenic insertions of the self-eliminating transgene will be established.

### Example 14: Programmed Transgene Self-Elimination in Tribolium

Beetles homozygous for the self-eliminating transgene from Example 13 will be reared in the absence of tetracycline (or subject to heat shock) to activate the expression of the HEG. Progeny will be analyzed and scored for restoration of eye/body pigmentation along with loss of the fluorescent marker, indicating successful transgene self-elimination. Beetles will be kept off tetracycline (or heat shocked each generation) for at least three generations to establish the rate of transgene loss. Molecular analyses such as PCR and sequencing will confirm the form of repair. Empirical data will be obtained on the rate of pre-programmed transgene loss from an important agricultural pest and model genetic species.

### Self-Eliminating Transgenes from a Highly Invasive Weed

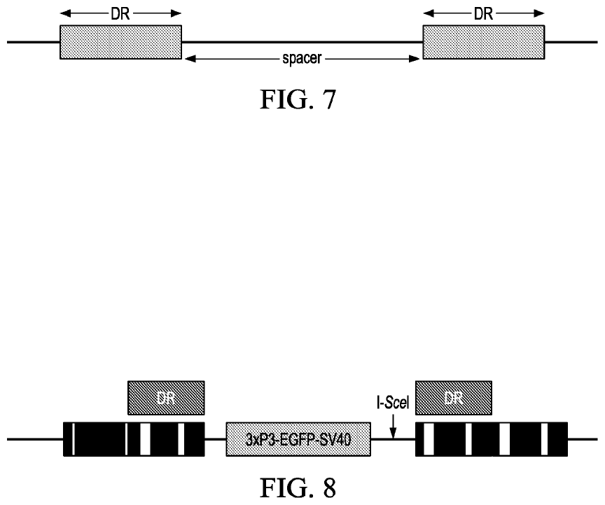

Amaranthus palmeri is a major pest to cotton and soybean production in the United States. The emergence of glyphosate resistance in this noxious weed combined with its obligate sexual reproduction (plants are either male or female only) makes it an excellent candidate for gene drive-based approaches. Transgenic A. palmeri will be generated with the self-eliminating transgene construct and to establish that the system can be effective in highly invasive weeds. FIG. 5 shows constructs for evaluating gene deletion in A. palmeri using a self-excising nuclease construct.

### Example 15: Establishment of a Gene Transformation for A. palmeri

A gene transformation methodology for A. palmeri will be developed. Two types of gene transformation techniques (callus and female gametophyte infection with Agrobacterium) will be developed. Two sets of vectors harboring the β-glucuronidase (GUS) gene under different promoters will be generated using synthetic biology or conventional cloning methods. A constitutive promoter (CaMV35S), heat-shock inducible, ethanol inducible, and dexamethasone inducible system will be used for the construction. For each construct, two sets of vectors (a set in a high-copy, small plasmid for a transient expression and a set in a binary vector for Agrobacterium-mediated transformation) will be generated. For gene transformation, either calli derived from a mature embryo or female gametophytes will be infected with Agrobacterium harboring the plasmid containing a constitutive or inducible promoter driving GUS. A direct infection of female gametophyte will be tested in parallel. As a positive control, A. hypochondriacus plants will be transformed using protocols known to those skilled in the art. The potential transformants will be selected with an antibiotic marker, followed by a PCR analysis to confirm the transgene integration. For plants transformed with an inducible promoter, the promoter activities will be assayed by adding increasing concentrations of induction reagents. Plants carrying the transgene will be regenerated, and the GUS activity will be confirmed. A methodology will be established to transform A. palmeri, as well as identification of the inducible promoter that work well in this species.

### Example 16: Evaluation of Inducible Transgene Deletion in Am. palmeri

The activity of DSB-induced transgene deletion in A. palmeri will be examined. Deletion of an antibiotic-resistance marker gene, induced by a homing endonuclease, has been reported in a model plant Arabidopsis. Transgene deletion in A. palmeri and the dominant repair pathway in this species will be examined. A set of binary vectors harboring an endonuclease (HEG or CAS9) and 35S promoter-GUS reporter sequence flanked by the recognition sequences will be constructed. In addition to the recognition sequences, the constructs will carry a set of direct repeat sequences outside of the nuclease recognition sequence. The nucleases will be expressed under an inducible promoter, as evaluated in Example 15. It is predicted that the excision efficiency will be influenced by the distance between the two nuclease recognition sequences. The self-excision of the nuclease sequence will be tested by placing the two recognition sequences flanking both the nuclease and reporter expression construct. This increases the distance between the two sequences from −3 kb to −5.5 kb. Once transgenic lines are established, leaf discs will be isolated from the transgenic plants and cultured under non-inducible and inducible conditions. Gene excision activities will be measured by the loss of GUS reporter activity within the leaf discs. In addition, PCR will be performed using primers that recognize the transgene, as NHEJ-based repair and HR-based repair will produce different fragment sizes.

It is anticipated that inducible gene deletion will be observed. The efficiency of inducible gene deletion system will be established, as well as the relative frequency of NHEJ- and HR-based repair events. In case NHEJ-based repair predominates and no targeted gene insertion is observed, siRNA for ku70 or DNA ligaseIV genes may be included to promote HR-based repair.

### Example 17: Establishing Transient Gene Expression System in A. palmeri

Another embodiment of the invention will be demonstrated with a self-excising gene drive unit with a targeted gene insertion in A. palmeri. A system to transiently express an exogenous gene in protoplasts, with the ability to then regenerate the entire plant, has several merits. Firstly, the turnaround time for transgene expression using such a method is much faster than gene transformation (<1 wk versus 3 months), enabling much higher throughput. In addition, macromolecules such as proteins and RNAs can be delivered to the cells during the procedure, enabling the donor sequence for HR-based repair, as well as reagent that enhances HR-based repair to be delivered during gene transformation. Protoplasts will be prepared from young mesophyll cells of A. palmeri, using protocols known in the art for other species as a starting point. This experiment will demonstrate that a transgene can be expressed in mesophyll protoplasts from A. palmeri. Once the protoplast transformation protocol is further optimized, it will be used to optimize the targeted gene deletion protocol. For this experiment, appropriate target genes on the A. palmeri genome will be identified. Since no genome or RNAseq data is available for this species, RNAseq on RNA samples extracted from representative tissues (roots, leaves and flowers) will be performed and used to determine potential target genes. The ideal target genes will allow a simple screen for loss-of-function, such as loss of color in certain tissues or loss of an enzymatic activity. Candidate targets include, but are not limited to, chalcone synthase (CHS), whose loss-of-function results in yellow seed coat, and alcohol dehydrogenase (ADH), which results in the loss of alcohol dehydrogenase function. Target genes from A. palmeri will be mined from the RNAseq data. It is known in the art that the genome of a closely related species, A. hypochondriacus, has a single, conserved CHS gene.

Next, the donor plasmid will be assembled by gene synthesis and/or conventional PCR-based cloning. A CRISPR/Cas9 construct carrying an appropriate sgRNA will be constructed using golden-gate cloning system for sgRNA. Both constructs will be introduced in the protoplasts using the methods optimized in Examples 15-16. The correct insertion of the transgene will be detected by a PCR reaction spanning the genome and transgene.

In parallel, a construct carrying the necessary components for self-deletion, flanked by the repeats of endogenous target sequences, will be constructed and introduced into protoplasts. This experiment will allow for evaluation of whether a much larger deletion compared to what is tested in Example 16 feasible. In addition, when combined with a technique to regenerate transgenic plants from protoplasts (as known in the art for other species), the protoplast mediate method offers a possibility of regenerating a whole plant that carries the transgene at the target locus without creating the second site carrying the nuclease construct. The possibility of inserting a relatively large construct will be tested. It is expected that there will be successful reporter expression in A. palmeri protoplasts and targeted gene insertion will be detected using PCR reactions, thus verifying targeted gene insertion, which is a prerequisite for self-eliminating gene drive strategy.

### Modeling the Self-Elimination of Transgenes in the Context of Population Reduction and Population Conversion Strategies

In order to determine whether the empirical data obtained in each of Examples 1 through 24 is sufficient to justify field-based trials and continued pursuit of the self-eliminating transgene technology, known continuous and stochastic models of gene drive will be updated to incorporate the additional parameters of spontaneous transgene loss with or without target site regeneration.

### Example 18: Inundative Releases to Achieve Population Replacement Modified to Incorporate Transgene Self-Elimination at a Range of Efficiencies

Modeling known in the art has shown that even in the absence of an active gene drive mechanism, the large scale inundative release of mosquitoes carrying a dominant anti-pathogen molecule can result in the fixation of the transgene in nature. However, such releases face the same predicament as do gene drive scenarios: how to test the effectiveness of the anti-pathogen mosquitoes in a real-world field setting without the permanent introduction of transgenic individuals into the wild. For example, if the anti-pathogen transgene(s) did not perform as expected, it would be optimal to remove the transgene from the wild (both to re-use any marker genes and to prevent the evolution of the pathogen in the case of a partially-effective anti-pathogen gene). Thus, strategies based on inundative release would benefit from a pre-programmed self-eliminating transgene. Known stochastic models will be updated to include the spontaneous reversion of transgenic individuals to wild-type at a variety of rates. For each rate, the model will show how long the transgene is predicted to remain in the population after releases stop, based a variables such as population size and inundative release rate.

### Example 19: Medea, Underdominance, and Other Threshold-Based Gene Drive Approaches Modified to Incorporate Transgene Self-Elimination at a Range of Efficiencies

Known modeling suggests that threshold-based gene drives will be harder to establish in nature, making them robust against accidental releases. Once established in a target population, such constructs may potentially be removed from the wild through the subsequent release of wild-type individuals, until the threshold is reached, further pushing the transgene out completely. However, it is possible that in the case of an abrupt end to a trial, remediative releases may not be possible. The self-elimination of a Medea or underdominance-based transgene would serve the same function, but would not require any remediation. The introduced transgene could potentially slowly disappear from the population until the threshold was reached, at which point it would be expected to rapidly disappear. The Medea and underdominance-based models known in the art will be updated to include the spontaneous reversion of transgenic individuals to wild-type at a variety of rates. For each rate, the model will show how long the transgene is predicted to remain in the population after releases stop, based a variables such as certain population size and initial release rate.

### Example 20: HEG-Based Chromosomal Shredding, Modified to Incorporate Transgene Self-Elimination at a Range of Efficiencies

HEG-based X-specific shredding has been developed for An. gambiae. CRISPR/Cas9 based targeting of other X-specific sequences or the overexpression of male-determining genes would yield the same practical result—the shift in population towards extreme male bias. Propagating such bias has been predicted through stochastic or continuous modeling known in the art to result in the local elimination of the target population. As it is unclear what “local” may mean in this context, it is possible that the introduction of an active gene drive construct capable of driving male bias might send a species (and any others capable of productive breeding) to extinction. To prevent this, particularly during the investigational and testing phase, the incorporation of self-eliminating transgene technology may substantially reduce the risk of an unintended global extinction event. The stochastic models and the continuous propagating wave model (reaction-diffusion) which includes the spontaneous reversion of male-biasing transgenes to wild-type at a variety of rates which are known in the art will be updated. For each rate, the model will show how long the male bias is predicted to remain in the population after releases stop, based a variables such as population size and initial release rate.

### Example 21: CRISPR/HEG Based Gene Drive Coupled with a Pre-Programmed Self-Eliminating Transgenes at a Range of Efficiencies

Recent reports that Cas9-mediated gene drive is highly efficient in Drosophila, An. stephensi and An. gambiae have raised hopes that mosquito populations may be rapidly converted to a plasmodium-resistant state, breaking the cycle of malaria transmission while also addressing concerns regarding control of transgenes once released. Known models suggest that such a gene drive system would quickly become established, even with the accidental release of just a few individuals. The current models will be adjusted to set a finite limit of the presence of the introduced transgene in nature. The adjustments will address, given certain release rates and spatial patterns, the spread of the gene drive system before self-elimination, as well as the parameter space whereby a self-eliminating transgene can allow sufficient drive to evaluate the technology in a field setting, while eventually overcoming the robust nature of the gene drive system and eliminating it from the study area.

### Example 22: External Stimulus-Triggered Transgene Elimination

Each of the previous scenarios assumes a constant rate of transgene elimination beginning immediately upon release, the so-called slow fuse model where the activating HEG is expressed at a low level all of the time. Models also will be modified to include a time restriction, whereby transgene self-elimination does not occur without the application of an external stimulus (either the removal or addition of a chemical agent).

### Example 23: Cas9-Resistant Genotypes

While all known work to date has been focused on the use of gene drive systems for the benefit of human health and prosperity, it is possible that as the technology matures it could be used with intent to do harm. A critical question then is whether the vulnerable species may be protected from an invading gene drive system proactively (without knowledge of where it might attack the genome), or in direct response to a detection. Models also will be modified to address this issue and include the introduction at various times pre- and post-establishment of an active Cas9-based gene drive construct in the context of genotypes that transcriptionally/post-transcriptionally silence Cas9 expression.

### Example 24: Feedback from Experimental Data to Further Inform the Models

As data are generated, a number of other parameters such as release size, spatial dimensions, population structure, migration rates, release timing, and so forth, may be added to the models to further predict the performance of self-eliminating transgenes. Each set of models can be parameterized with the life history traits, generation time, reproductive capacity and empirical data obtained for each organism.

At the conclusion of Examples 18-24, rigorous predictions for how pre-programming transgene elimination may affect various gene drive scenarios will be available for use in preparation for field-based trials.

### Example 25: Determine the Contribution of Length and Spacing of Direct Repeats to Self-Elimination

The experiments in this Example make use of both the dipteran model organism Drosophila melanogaster and the disease vector Ae. aegypti. Using both systems is important to fully evaluate the self-elimination mechanism to control gene drive transgenes. With its short generation time, ease of rearing and a suite of highly tractable genome manipulation tools, using Drosophila allows for the expansion of the scope and scale of these experiments (particularly the number of transgene varieties that can be tested) well beyond what is practical for mosquitoes. In addition, successful gene drive approaches are readily available for Drosophila but have not yet been developed for Ae. aegypti. On the other hand, relying entirely on Drosophila would not be ideal either, as it these experiments should be applicable to disease vectors. Together, these experiments highlight similarities and differences between these two dipterans, in turn informing how this technology might be applied to other species such as Anopheline vectors of malaria or Culex vectors of West Nile virus.

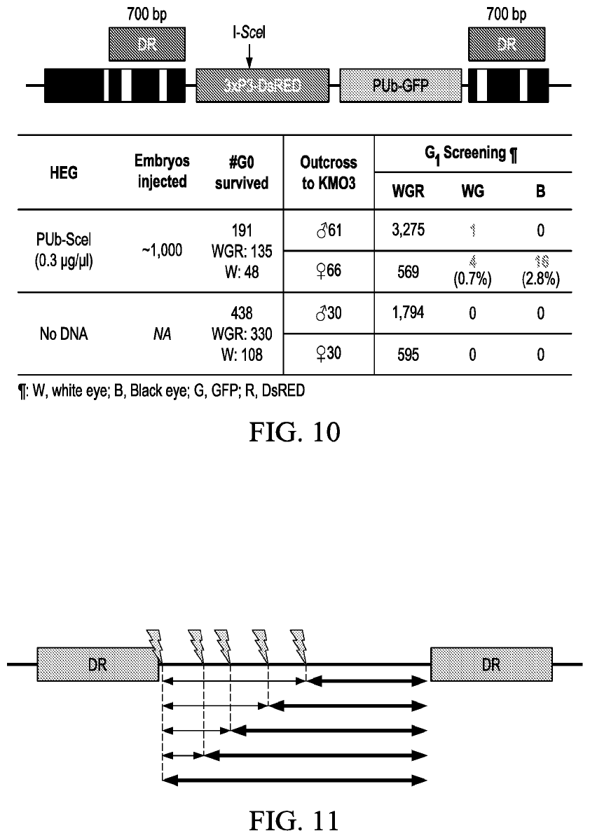

Intrachromosomal deletions mediated by the SSA pathway can result in deletions of at least 80 kbp at high efficiency with the length of repeats and distance of separation strongly influencing this mode of repair in yeast, flies and vertebrate cells. The length of sequence that can be eliminated in turn dictates the length a minimal nuclease-based gene drive system along with any visual markers and anti-pathogen genes can be. Previously reported deletions of more than 2,000 bp using very short (200 bp) repeats have been shown by the inventors, while previous work in Drosophila has been limited to short repeats (˜250 bp) and spacers (<2.5 kb). Thus, an analysis of these parameters (FIG. 7) at the scale of current gene drive constructs (10-16 kbp) is important to realizing the full potential to engineer self-eliminating gene drive systems.

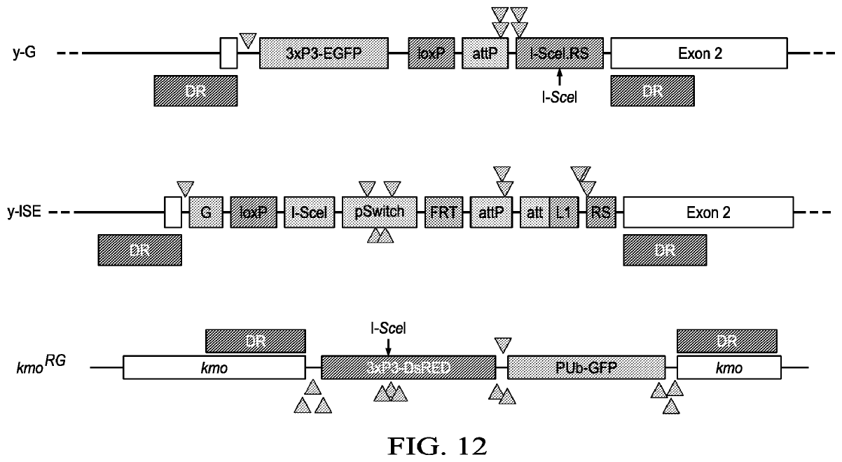

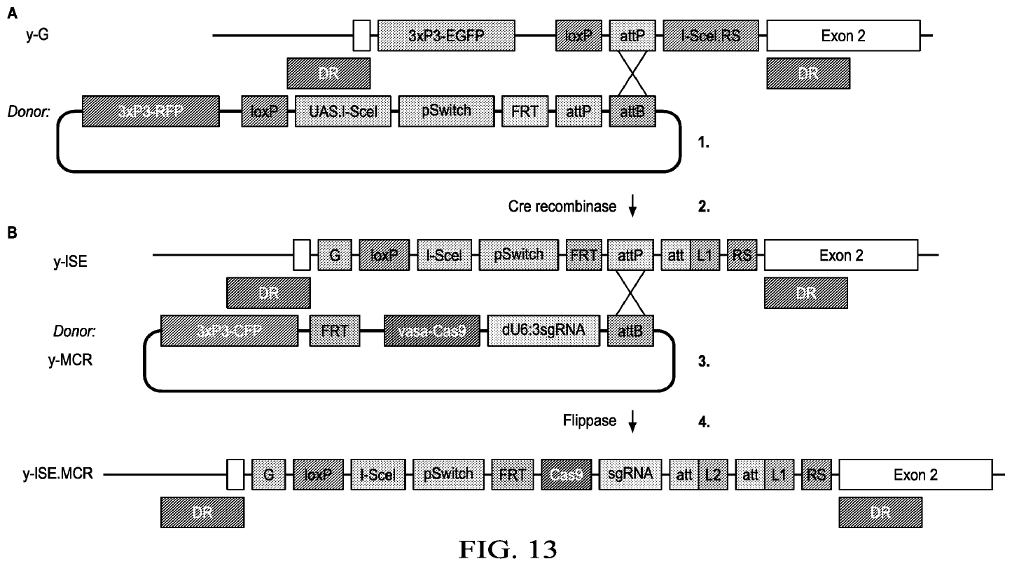

An EGFP transgene flanked by directs repeats of three different lengths (30 bp, 250 bp, 500 bp) was successfully engineered into the Drosophila yellow (y) gene (FIG. 8). Introduction of a DSB, mediated by the homing endonuclease I-SceI, between these repeats results in SSA-based repair, loss of EGFP fluorescence, and restoration of wild-type body pigmentation. Indeed, treatment with the I-SceI nuclease resulted in rates of SSA that could be directly correlated with the length of the direct repeat (FIG. 9). While the initial construct contained only ˜1.5 kb of sequence between the direct repeats, SSA-based transgene elimination was also observed with 7.2 kb of spacing between direct repeats 500 bp in length.

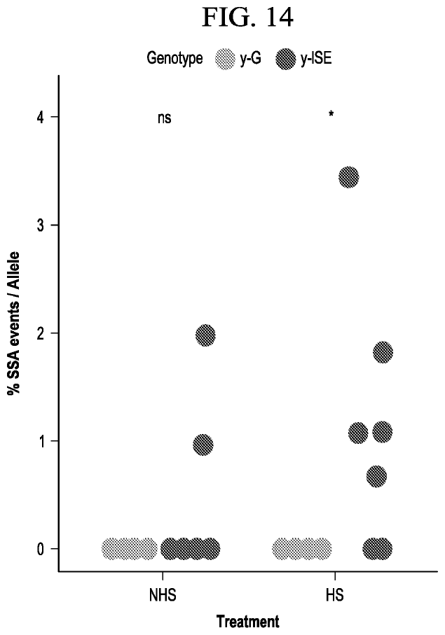

Similarly, direct repeats as short as 34 bp were able to eliminate ˜1.5 kbp of intervening sequence in Ae. aegypti, but not ˜2.7 kbp, while a repeat length of 195 bp enabled the elimination of ˜2.4 kbp of transgenic sequence. Two additional transgenic strains were generated, whereby a set of two marker genes (DsRED and EGFP) were integrated into the Ae. aegypti kmo gene flanked by direct repeats of 200 bp or 700 bp. Introduction of a DSB in between the 700 bp repeats resulted in the elimination of both marker genes (˜4 kb) and the restoration of wild-type eye pigmentation (FIG. 10).

These data show that extending the repeat length permits more efficient transgene elimination over longer intervening sequences. The experiments detailed below will methodically analyze the effects of repeat length and repeat distance on transgene elimination in both the model dipteran Drosophila melanogaster and the disease vector Ae. aegypti. The ease of generation, maintenance, and experimenting with transgenic fly strains permits testing of many more combinations than would be possible in the mosquito alone. As work in Drosophila proceeds more rapidly, results obtained from experiments in the genetic model organism will in turn refine the design of constructs used in Ae. aegypti, as described below.

### Determining the Effect of Direct Repeat Length on SSA-Based Self-Elimination in Flies.

It is likely that there is a maximum direct repeat length, at which point further increases in SSA mediated repair of the dsDNA break will not occur. With an extremely tractable fly model, it will be determined if and where this point exists, at least with regard to sizes where direct repeats can practicably be employed to control nuclease-based gene drives. To do this, CRISPR/Cas9 will be used to knock-in a series of transgenes into existing fly strains containing direct repeats of varying length (FIG. 8). Additionally, new fly strains will be generated with repeat lengths ranging between 1 and 5 kbp. New transgene sequences will be engineered to include a DsRED sequence with an I-SceI recognition site that will both serve as a marker of transformation and enable the scoring of NHEJ events, which was not possible in the experiments detailed above. Expression of both EGFP and DsRED in G₁ progeny will suggest stable integration of the donor construct, to be confirmed through PCR analysis and sequencing. Once established, I-SceI will be provided either by plasmid or through crossing with an I-SceI expressing transgenic line that has been generated. While NHEJ-mediated gene disruption at the I-SceI cut site can be scored through loss of DsRED fluorescence (y−, EGFP+, DsRED−), restoration of wild-type body pigmentation in G₁ progeny can only occur following SSA-based repair using the direct repeats (y+, EGFP−, DsRED−). Flies not exposed to I-SceI will serve as controls. These experiments will allow better definition of the relationship between direct repeat length and SSA-based repair at a scale and resolution not practical in mosquitoes.

### Determination of the Effect of Direct Repeat Length on SSA-Based Self-Elimination in Mosquitoes.

These experiments will begin by using the two strains already developed (200 bp and 700 bp repeats) to more fully analyze the rate of SSA-based repair in Ae. aegypti. Additionally, CRISPR/Cas9-stimulated gene insertion will be used to generate two additional transgenic Ae. aegypti strains with provisional direct repeat lengths of 1500 bp and 3000 bp. However, the length of direct repeats will be finalized based on data obtained from Drosophila as detailed above. Each new integration will be identified by DsRed and EGFP, with confirmation obtained through PCR analysis of the integrated transgene. Once established, embryos from each line will be injected with a plasmid-based I-SceI expression construct. Survivors will be crossed with a kmoΔ4 strain and progeny screened for fluorescence and eye pigmentation. While end-joining repair can result in the loss of DsRED fluorescence through disruption of the DsRED ORF, restoration of eye pigmentation can only occur following SSA-based repair. Thus, the rate of both SSA-based repair (Black eye, EGFP−, DsRED−) and NHEJ-based repair (White eye, EGFP+, DsRED−) can be determined by scoring G1 progeny. As the repeat length increases, likely so will the number of SSA-based events; inversely, NHEJ events are expected to decrease. Repair events will be confirmed through PCR and sequencing. Uninjected embryos or those injected with a non-functional I-SceI (no ATG) will serve as a negative control to estimate spontaneous transgene elimination rates.

### Determination of the Effect of Distance Between Repeats on SSA-Based Self-Elimination in Flies.

Fly lines as detailed above that demonstrate the highest efficiency of SSA-based transgene elimination will be selected, and the ϕC31 integrase system will be used to incorporate 5-10 additional spacer sequences between the direct repeats. The blue fluorescent protein, mTagBFP, will serve as the marker of transformation into the existing lines. The additional transgenic sequences will increase spacing between the direct repeats from ˜1.5 and 7.2 kbp to ˜15-21 kbp. Integration of each donor construct will be confirmed through PCR and sequencing over attL and attR flanking regions. Once each line is established, I-SceI will be expressed as described above, either from a plasmid or via an independent locus. Flies not injected with plasmid expressing functional I-SceI will again serve as controls. These experiments will permit rapid determination of the relationship between the type of DSB repair that occurs and spacing of the direct repeats. For example, increasing the distance between direct repeats might favor DSB repair pathways other than SSA, decreasing the frequency of transgene elimination. If true, then any strategy for eliminating a nuclease-based drive with this technology may need to be optimized for the size of the construct, which might vary with cargo size, by altering direct repeat sizes or location of DSBs. However, answering these questions in Drosophila will permit determination of how much time, effort and resources should be devoted to optimization of this parameter in the non-model disease vector, Ae. aegypti.

### Determination of the Effect of Distance Between Repeats on SSA-Based Self-Elimination.

Dependent on the results above, two of the four transgenic strains of Ae. aegypti (those already in hand and/or those generated above) will be selected and the ΦC31 integrase system will be used to incorporate an additional transgene via attP:attB recombination, with transformants identified with mTagBFP. The ΦC31-integrated transgenes will increase the spacing between the direct repeats from ˜4 to 8 or 16 kbp; integrations will be confirmed by PCR on genomic DNA over the resulting attL and attR flanking regions. Once established, embryos of each transgenic strain will be injected with plasmid expressing I-SceI as detailed above. Survivors will be crossed with kmoΔ4 mosquitoes and progeny scored for eye color, EGFP, mTagBFP and DsRED fluorescence. Once again, successful SSA-based repair will eliminate all fluorescent markers and restore eye pigmentation while end-joining repair will result in the loss of DsRED only. As in flies, increasing spacing between direct repeats will likely reduce the rate of SSA-based repair. If results in the fly indicate little or no relationship between the distance of the direct repeats and SSA-based elimination, a single line with the largest spacer (16 kbp) may be generated to confirm findings in the model organism.

The data above demonstrates effective SSA-mediated transgene elimination in both Drosophila and Ae. aegypti. As an alternative to injection of I-SceI plasmid into embryos, transgenic Ae. aegypti strains may be generated that express I-SceI and perform crosses to induce DSB formation. Transgenes or constructs may become unstable if the direct repeats become too large. Rather than being a problem, this could indicate the possibility of a self-eliminating transgene that does not even require a second nuclease, substantially simplifying the approach to controlling and eliminating gene drives from a population.

### Example 26: Determination of the Contribution of Distance Between DNA Break Induction and Direct Repeats to Transgene Self-Elimination

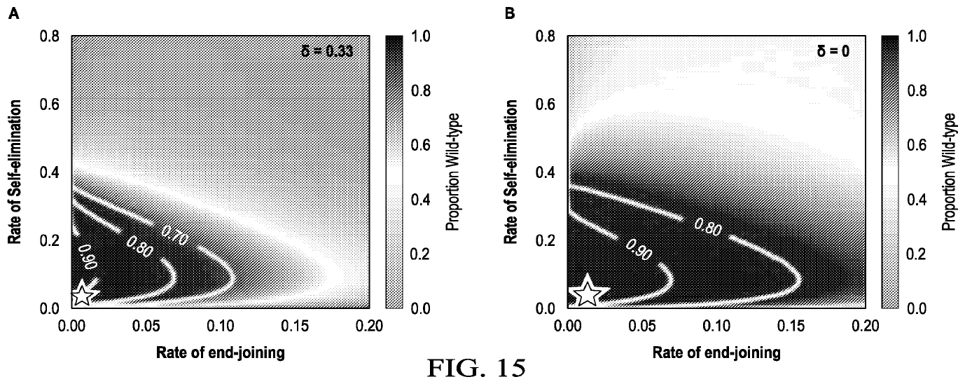

One of the first steps in SSA-based DNA repair is resection of one or both ends of the break to generate single-stranded tails used in a homology-based search. However, if resection ceases prior to revealing at least one direct repeat, SSA may not occur. Thus, the distance between the site of DSB induction and one or both direct repeats represents an important parameter for the development of a self-eliminating strategy (FIG. 11). In this Example, advantage is taken of the ease of developing new CRISPR/Cas9 guide RNAs to probe the effects of moving the site of DSB induction closer or farther from each of the two direct repeats. While the initial approach utilized a single I-SceI recognition event to trigger transgene elimination, it is relatively simple to engineer multiple nuclease recognition sites as part of the transgene self-elimination approach. Using multiple nuclease recognition sites will likely increase the rate of transgene elimination. It will be observed either: (1) an additive effect, where each DSB introduced is associated with an independent probability of being repaired via NHEJ or through SSA-mediated transgene elimination. Thus, more DSBs would mean more chances for the transgene to be removed. Or (2), a synergistic effect, for example if multiple simultaneous DSBs in close proximity to each of the direct repeats increases the rate of transgene elimination beyond what would be expected if each acted alone.

In the inventors' initial observation of SSA-based transgene elimination in Ae. aegypti, the site of DNA break induction was between 50-150 bp from the start of one of the repeats. In the updated transgenes with 200 bp and 700 bp repeats, the site of DSB induction (I-SceI target site) was ˜300 bp from one of the direct repeats. Multiple transgenic lines have been developed for assessing transgene elimination in Drosophila melanogaster, with the site for DSB induction 20 bp from the nearest direct repeat.

### Distance Between DSB Induction and Direct Repeat in Flies and Mosquitoes.

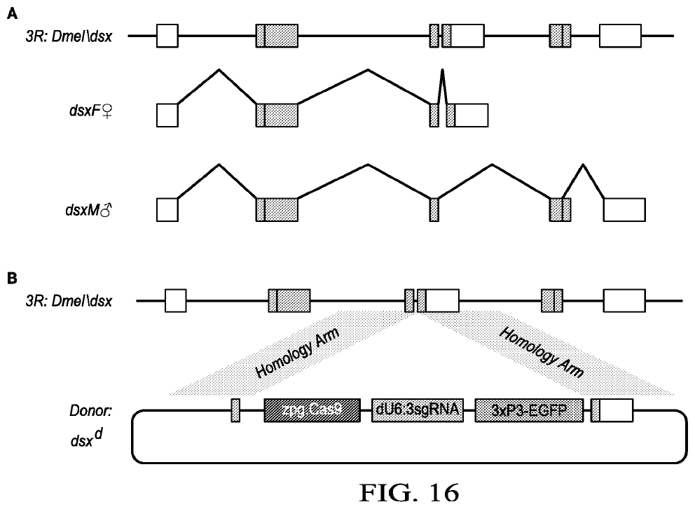

As described above, Drosophila strains have been generated with transgenes that vary with respect to the size of the direct repeats (30 bp, 250 bp or 500 bp) and intervening sequences (1.5 kbp or 7.2 kbp). A series of sgRNAs will be designed that target the intervening sequences (FIG. 12). These sgRNAs will be used with CRISPR/Cas9 to introduce DSBs at various distances from the direct repeat sequences. Likewise, a series of 10-20 sgRNAs will be generated in 4-6 groups (3-4 sgRNAs per group) targeting the DsRED or EGFP transgenes of Ae. aegypti kmoRG strains already developed and described above containing 200 bp or 700 bp direct repeats (FIG. 12). For both flies and mosquitoes, one group of sgRNAs will be coincident with the I-SceI target site, allowing the comparison of the effectiveness of transgene elimination using CRISPR/Cas9 (which generates a blunt DSB) and I-SceI (which generates a 3′ overhang). The same sgRNAs can be used to induce DSBs with either blunt (Cas9) or sticky ends (Cas9-nickase) by substituting different Cas9 variants, permitting assessment of both the nature of the DSB, as well as the distance from the direct repeat, on the efficiency of SSAbased repair. Using multiple sgRNAs per transgene position will allow better separation of the contribution of the target site location, as some variability in sgRNA performance is expected. Some sgRNA groups will be targeted very close to the direct repeats (0-50 bp); others will be further from the direct repeats, with one located as close to the center of the transgene set as possible. All sgRNAs will first be validated for effectiveness by injection into pre-blastoderm embryos, followed by DNA extraction, PCR and high-resolution melt analysis. Designing multiple sgRNAs for each location targeted in the transgene will ensure assessment of the contribution of DSB location while controlling for variability in the performance of any single sgRNA. For flies, validated sgRNAs will be injected into homozygous embryos (y-G with 250DR or 500DR or y-ISE with 250DR or 500DR) along with a source of Cas9. Surviving individuals will be mated and G1 progeny scored for loss of fluorescent markers and restoration of wild-type body pigmentation. The genotypes of a subset of the phenotypically scored flies will be confirmed by PCR and sequencing. The rates of SSA-based excision measured in each of these groups will be compared to those mediated by I-SceI in previous experiments. For mosquitoes, each sgRNA will be injected into kmoRG/kmoRG embryos along with Cas9 protein, with surviving individuals mated with white-eyed kmoΔ4 strain mosquitoes. As detailed above, transgene elimination will result in the loss of both fluorescent markers and the restoration of pigmentation in the eye. A subset of each phenotypic class will be subject to PCR/sequencing to confirm the associated genotype. Progeny will be scored from at least 70 fertile founders for each sgRNA and compare the rate of transgene elimination to that obtained for I-SceI.

### Number of Induced DSBs and Transgene Elimination in Flies and Mosquitoes.

For flies, effective sgRNAs identified above will be combined and injected with Cas9 into homozygous embryos (y-G 250DR; 500DR or y-ISE 250DR; 500DR). The progeny of surviving embryos will be mated and screened as described above, with the genotypes of a subset of the phenotypically scored flies again confirmed through PCR and sequencing. For mosquitoes, at least 4 pairs of effective sgRNAs from independent groups developed above will be delivered together with Cas9 protein into kmoRG embryos, with surviving individuals mated with white-eyed kmoΔ4 strain mosquitoes as before. Again, progeny will be screened for DsRED, EGFP and eye pigmentation, with the expectation that transgene elimination will result in loss of both fluorescent markers and restoration of eye pigmentation. As above, a subset of each phenotypic class will be subject to PCR/sequencing to confirm the associated genotype and progeny will be scored from at least 70 fertile founders for each sgRNA pair and the rates of transgene elimination compared to that obtained for each sgRNA alone above. sgRNA pairs will be selected based on effectiveness in mediating transgene elimination when injected individually, and proximity to the direct repeats. Rates of self-elimination will likely be higher when sgRNAs close to each direct repeat are combined. It will be particularly interesting to determine if inducing DSBs at regular intervals, or simultaneous targeting of multiple locations in close proximity to the direct repeats at both the 3′ and 5′ homology arms, can increase the efficiency of SSAbased repair.

These experiments do not depend on the generation of any new transgenic mosquito or fly strains, and can be effectively completed with the existing strains already developed. Strains developed above with larger spacer regions between the direct repeats may be incorporated into these experiments as well, further enriching the dataset. The data obtained here is useful not just for transgene self-elimination, but for any genome engineering approach where naturally occurring repetitive sequences are present around a region of interest.

### Example 27: Evaluation of the Pre-Programmed Elimination of an Active Gene Drive

Genetic strategies to control dengue based on the release of sterile, transgenic individuals are currently underway and have been successful where attempted. These strategies provide effective mosquito control only as long as releases continue, and thus represent a long-term financial and administrative commitment that must be maintained even in the absence of continued transmission. For this reason, gene drive systems that permanently confer on the target population a refractory state have long been sought after. Once released, such systems cannot be contained, and this limitation, along with the unknown effects on natural ecosystems of the introduced transgene, likely precludes effective field-testing of engineered strains. Thus, there is a compelling argument for technologies that permit deployment and evaluation of gene-drive based approaches while simultaneously being self-limited and in essence, biodegradable.

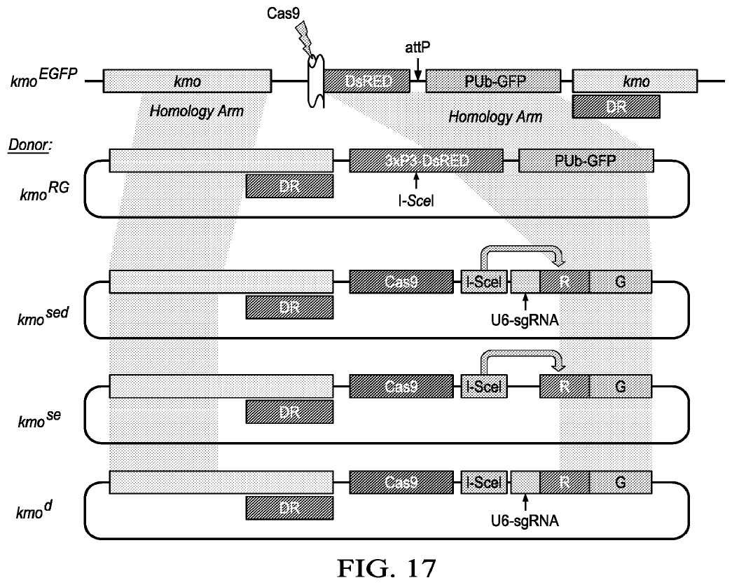

Building on the preliminary data presented above (FIG. 9), multiple D. melanogaster lines containing a self-eliminating transgene (constructs contain both an active nuclease and ISceI site) were generated with direct repeats of varying length (30 bp, 250 bp and 500 bp). The strategy for assembling these constructs is shown in FIG. 13. ϕC31-mediated recombination (step 1) was used to insert I-SceI under the control of an inducible heat shock promoter into the previously constructed y-G transgenic lines (FIG. 8 and FIG. 12). Subsequent excision of RFP, a marker of transgenesis, and ampicillin gene via Cre-lox recombination (step 2) resulted in the y-ISE strains (30DR, 250DR, and 500DR) illustrated in FIG. 12 and FIG. 13.

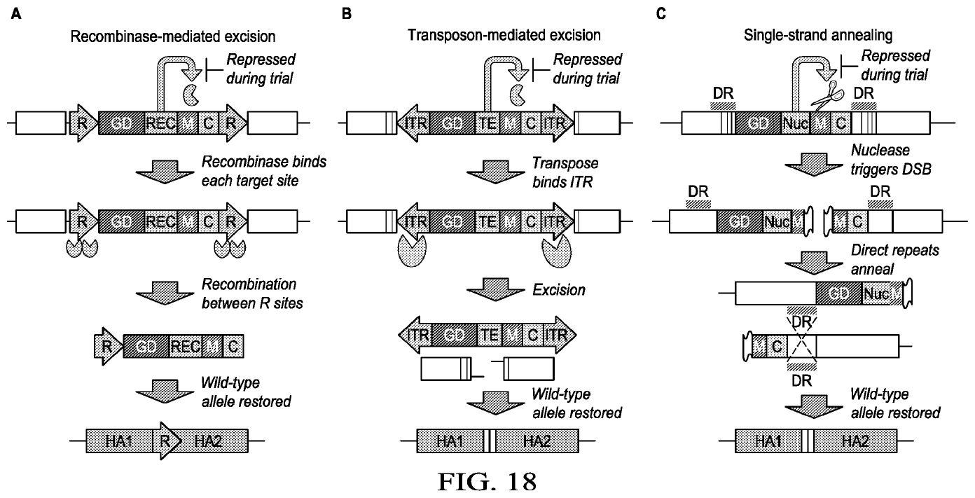

Heat shocking the y-ISE 250DR strain resulted in SSA-based self-elimination of the transgene in a subset of flies exposed to the higher temperatures, which was scored by a loss of EGFP and reversion to wild-type body color (y+G−; FIG. 14). A silent mutation (TGG→TcG) incorporated into the transgene to destroy the gene drive sgRNA target site, leaves a single nucleotide scar that serves to differentiate SSA-mediated elimination of the transgene from wild-type yellow alleles, which would be otherwise indistinguishable. The presence of this single nucleotide scar in y+G− flies confirmed that precise elimination of the transgene had indeed occurred. Importantly, SSA-mediated elimination of the transgene was not observed in y-G control flies lacking the UAS.hsp70.I-SceI element, indicating that the DSB induced by the homing endonuclease was necessary for SSA-based elimination of the transgene (FIG. 14). These results clearly demonstrate that a transgene can be programmed to self-eliminate.

In order to extend the findings demonstrating the programmable self-elimination of a transgene to an active gene drive, the previously described CRISPR/Cas9-based y-MCR gene drive was reconstructed. This gene drive system is based on homology-dependent integration into the X-linked yellow locus, converting a heterozygous recessive loss-of-function mutation in female flies into a homozygous mutant phenotype (y−), yellow body color. Through this process of autocatalytic allelic conversion, which has been termed a mutagenic chain reaction (MCR), the transgene converts the target population from the wild type y+ to the mutant y-phenotype. Table 3 summarizes the genetic transmission of the y− phenotype through two generations after mating of flies harboring the y-MCR transgene. A total of five G0 parents were identified (F9, F12, F17, F33, and M19) for these experiments, four females and one male. When the G0 parents were outcrossed with y+ flies they produced y− progeny that were scored as likely carrying the y-MCR construct, of which a subset was tested for skewed inheritance of the y-MCR construct. For example, four female and one male progeny of F33 was tested for propagation of the y-MCR transgene by outcrossing to y+ flies and scoring G2s for a y-phenotype. While the results of this analysis are summarized in Table 3, it is worth noting that the percent of female y-MCR progeny, 93.3%, and the percent of allelic conversion by homology directed repair (HDR) estimated from female progeny, 98.7%, are remarkably similar to the percentages previously reported (Gantz and Bier, Science 348:442-444, 2015), which were 97.3% and 94.5%, respectively. Additionally, the lone outcross involving a G1 male parent, where all female progeny would be expected to inherit an X-linked y-MCR construct, did indeed demonstrate HDR conversion with 100% of female progeny exhibiting a y− phenotype (Table 3). Also similar to the results previously reported, some instances where a y allele inherited from a y-MCR parent escaped allelic conversion was observed, presumably because the allele contained a nucleotide change at a locus homologous to the sgRNA PAM site, a so-called resistance allele (Table 3; S. No. 11).

TABLE 3
Summary for genetic transmission of a y- phenotype in two generations of y-MCR flies.

| | | | | | | | | | | | | | [symbol] 2(X [symbol] 0.5N)/ |
|---|---|---|---|---|---|---|---|---|---|---|---|---|---|
| | | | | | | | | | | | | | N [symbol] 100 |
| | | | | | | | | | | | | | HDR germline |
| | | | G2s | | | | | | | N | X | X/N [symbol] 100 | conversion |
| | y- mosaic | y- | y- | | y [symbol] | | Mosaic | | | Total G2 | Total HDR | % y [symbol] MCR [symbol] | rate(%) |
| S. No. | G0 | G1 | ♀ | ♂ | ♀ | ♂ | ♀ | ♂ | Total G2 | ♀ | ♀ | ♀ | ♀ |
| 1 | 9♀ | 1♀ | 2 | 2 | 0 | 2 | 0 | 0 | 6 | 2 | 2 | 100 | 100 |
| 2 | 12♀ | 1♀ | 9 | 4 | 0 | 0 | 0 | 0 | 13 | 9 | 9 | 100 | 100 |
| 3 | | 2♀ | 11 | 10 | 0 | 0 | 3 | 0 | 24 | 14 | 14 | 100 | 100 |
| 4 | 17♀ | 1♀ | 14 | 15 | 0 | 0 | 0 | 0 | 29 | 14 | 14 | 100 | 100 |
| 5 | 33♀ | 1♀ | 18 | 10 | 0 | 0 | 0 | 0 | 28 | 18 | 18 | 100 | 100 |
| 6 | | 2♀ | 21 | 11 | 0 | 0 | 0 | 0 | 32 | 21 | 21 | 100 | 100 |
| 7 | | 3♀ | 6 | 6 | 0 | 0 | 1 | 0 | 13 | 7 | 7 | 100 | 100 |
| 8 | | 4♀ | 16 | 11 | 0 | 0 | 1 | 0 | 28 | 17 | 17 | 100 | 100 |
| 9 | | 1♂ | 8 | 0 | 0 | 13 | 0 | 0 | 21 | 8 | 8 | 100 | 100 |
| 10 | 19♂ | 1♀ | 15 | 14 | 0 | 0 | 0 | 0 | 29 | 15 | 15 | 100 | 100 |
| 11 | | 2♀ | 15 | 9 | 1 | 0 | 0 | 0 | 25 | 16 | 15 | 93.75 | 87.5 |
| 12 | | 3♀ | 9 | 8 | 0 | 0 | 0 | 0 | 17 | 9 | 9 | 100 | 100 |
| | | Total | 144 | 100 | 1 | 15 | 5 | 0 | 265 | 150 | 149 | 99.3333 | 98.66666667 |

[symbol]

indicates data missing or illegible when filed

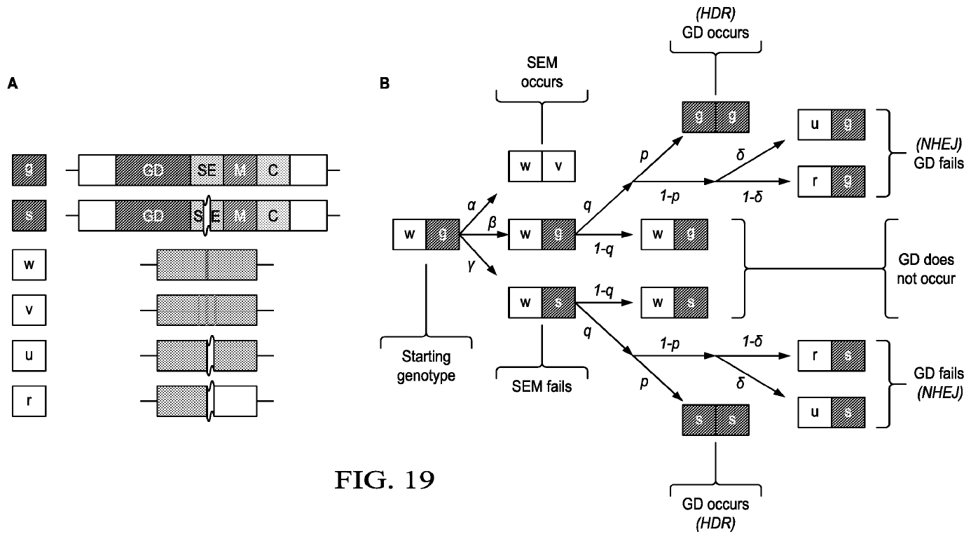

Separately, deterministic models of gene drive have been generated to determine the effect of self-elimination on the spread and long-term stability of a CRISPR/Cas9-based gene drive in a target population. In both cases, optimal rates of transgene self-♀♂ elimination range from just greater than 0 to about 20%, while tolerating rates of failure of the self-elimination mechanism as high as 5-10% (FIG. 15, panels A and B). Thus, these models predict that the rates of self-elimination currently observed are sufficient to control a CRISPR/Cas9 gene drive, no matter the target gene. While at first glance it might seem like the rate of self-elimination would need to be substantial, this is not the case. Essentially, the self-elimination mechanism creates an allele that is both resistant to the gene drive and has wild-type fitness—this is enough for selection to act on.

### Self-Elimination to Control a Yellow Gene Drive System in Flies.

Now that both the self-eliminating transgene and the MCR-based gene drive has been validated, the two will be combined. The y-MCR construct will be recombined into existing y-ISE transgenic lines through engineered attP/attB sites (FIG. 13, steps 3 and 4). Following integration, the CFP (marker of transgenesis) and ampicillin genes will be excised through FLP-FRT recombination as described above, creating multiple fly lines containing a self-eliminating gene drive construct (y-ISE.MCR) and direct repeats of varying length, which will be determined by, but not dependent on, the studies detailed above. Integrations will be confirmed by PCR and sequencing. Following the establishment of homozygous stocks, expression of I-SceI can be induced, either by heat shock, or through a pSwitch control element engineered into the construct (FIG. 13), which can be induced by ingestion of the chemical RU486. A series of y-ISE.MCR parents will be independently outcrossed with y+ flies and the genetic transmission of the y− phenotype monitored over multiple generations of y-ISE.MCR flies, as described above (Table 3). A subset of these outcrosses will be exposed to heat shock conditions or RU486. The progeny of both sets of crosses will also be scored for SSA-based elimination of the y-ISE.MCR construct by a loss of EGFP and reversion to wild-type body color (y+G−). Sequencing for the presence of the introduced single nucleotide scar in the y-ISE.MCR construct (discussed above), which creates a resistance allele following SSA-based elimination, will permit distinguishing of these events from naturally occurring end-joining based resistance alleles. Comparing the results of outcrosses held under standard conditions with those of the outcrosses in which I-SceI was induced, will permit demonstration of programmable self-elimination of an active gene drive.

### Self-Elimination to Control a DSX Gene Drive in Flies.

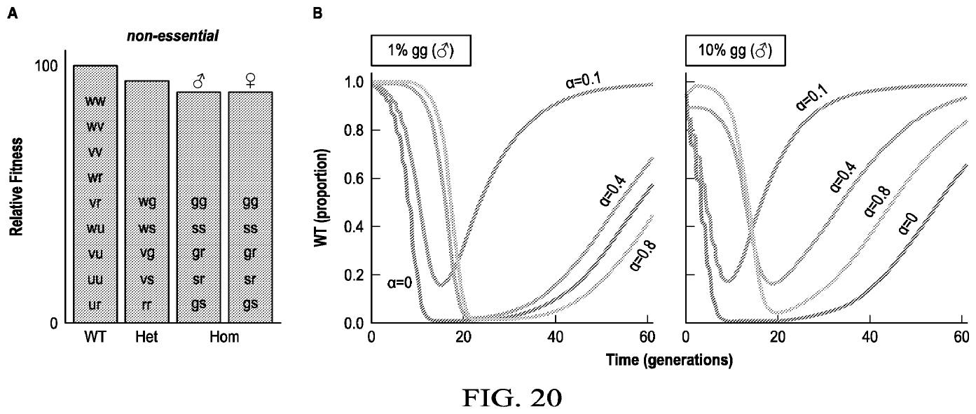

A CRISPR-Cas9 gene drive targeted to the female Anopheles gambiae doublesex gene was recently reported to reach 100% prevalence in caged mosquito populations, with no selection of resistance alleles. While resistant variants still arose, they were selected against due to the functional constraints associated with target sequence. Thus, application of the programmable self-elimination construct to a completely self-sustaining dsx gene drive would provide a much more rigorous test of the technology. Similar to An. gambiae, the somatic sexual differentiation of D. melanogaster is also regulated by the dsx gene, which has 6 exons (FIG. 16, panel A), of which the first three are common to both the sexes. The fourth exon is female-specific, while the fifth and sixth are male-specific. A dsx gene drive (dsxd) in Drosophila will be created and validated using a similar strategy, targeting the female-specific exon 4 (FIG. 16, panel B). In this construct, Cas9 will be under the control of the highly specific germline promoter, zero population growth (zpg), which has been shown to confer lower female fertility cost and reduce the formation of resistance alleles. Once validated, the y-ISE construct with direct repeats of optimum length will be recombined into dsx transgenic lines, and SSA-based elimination of the transgene assayed in experiments similar to those described above, with the exception that genetic transmission will be monitored solely with an eye-specific GFP phenotype rather than the y-phenotype. Introduction of the single nucleotide scar sequence will also be omitted, as this would introduce resistance alleles in the functionally constrained gene that would be selected against. Finally, larger scale population studies will be performed with both the active gene drive and self-eliminating gene drive, where the spread of the constructs, sex ratios, and population levels will be tracked over 10 generations.

### Evaluate the Baseline Rate of Transgene Elimination in Kmo^{EGFP} Gene Drive Mosquitoes.

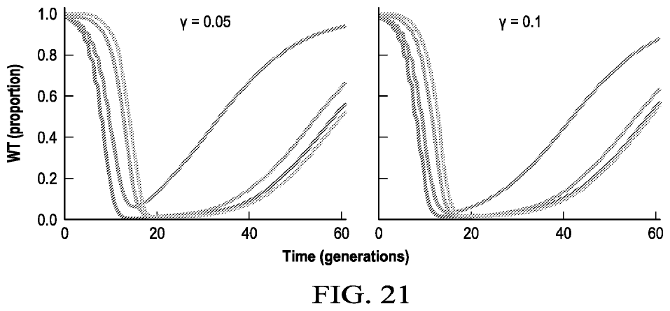

In order to translate the results from flies to disease vector mosquitoes, Cas9 protein and sgRNA will be injected into kmo^{EGFP} embryos along with a donor construct encoding the Cas9 ORF under the control of an Aedes germline promoter, an sgRNA under the control of an Aedes U6 promoter, the DsRED marker gene and a direct repeat to create strain kmo^{sed} (self-elimination drive; FIG. 17). An identical construct but without the U6-sgRNA cassette will be used to develop a negative control strain (kmo^{se}; self-elimination, but not drive), as well as with a version without the I-SceI target site (kmo^{d}; capable of drive but not self-elimination). To increase rates of homology-dependent integration, the injection mix will also include dsRNA targeting the end-joining factor ku70. Surviving individuals will be mated with the parental strain and progeny screened for DsRED. As the insertion site is pre-specified, only a single transgenic event is required for each construct. The resulting DsRed+EGFP+ progeny will be crossed by the parental strain to establish each line. Genomic DNA PCR/sequencing will be used to verify the landing site of the donor construct and the integrity of the components. To evaluate the baseline level of self-elimination in each line, kmo^{sed}, kmo^{d}, or kmo^{se} homozygotes will be crossed with the kmo^{Δ4} white-eyed mutant strain and score progeny as described in FIG. 10. At least three test crosses will be performed, with about 4000 progeny screened per cross. Self-elimination rates will likely be similar between kmo^{sed} and kmo^{se} strains, with little to no elimination observed from kmo^{d} mosquitoes.

### Self-Elimination to Control a DsRED Gene Drive in Mosquitoes.

Males homozygous for the kmo^{sed}, kmo^{se}, or kmo^{d} transgenes will be introduced into cages with kmo^{EGFP} males of the same age at various ratios (10:90, 25:75, and 50:50), along with kmo^{EGFP}/kmo^{EGFP} females. Following bloodfeeding and egg collection, a portion of the resulting embryos will be set aside for future genotyping, with a random subset hatched and all resulting larvae reared to adulthood without phenotypic scoring. This process will be continued for 10 generations. Allele frequencies for kmo^{sed}/kmo^{se}/kmo^{d} (white eye, EGFP⁺, DsRED⁺), kmo^{EGFP} (white eye, EGFP⁺) and kmo⁺ (black eye) will be determined for each generation. PCR/sequencing will be used to characterize a subset of each genotype. Both kmo^{d} and kmo^{sed} alleles will likely increase rapidly in the population due to gene drive, but kmo^{sed} alleles will subsequently decrease due to self-elimination. Both kmo^{d} and kmo^{sed} should generate traditional NHEJ-based drive-resistant alleles at the same rate, allowing control of these events. This ill generate empirical data on the rate of gene drive counterbalanced by transgene elimination in the germline of cage populations of Ae. aegypti, setting up large-scale cage trials and further optimization of the system. The gene drive described here targets a portion of the DsRED gene only.

The inventors have published several reports documenting the ability to use CRISPR/Cas9 technology to edit the Ae. aegypti genome. including several instances of efficient gene insertion. Germline promoters to drive Cas9 expression in Aedes aegypti have been described. While information concerning direct repeat length, spacing and the number and position of nuclease target sites will be used to inform construct assembly prior to generating transgenic strains as much as possible, these proof-of-principle experiments can be completed using the data already in hand if needed. The use of repressible (tet-off) or heat-inducible (HSP70) systems will be pursued to control the expression of the self-elimination nuclease. Upon escape from a contained laboratory or from a trial site the self-elimination mechanism could likely become activated, decreasing the likelihood of the transgene becoming established in nature. Unlike other proposed forms of molecular containment where active gene drive transgenes are split into multiple pieces (each of which can be collected and used to reform the gene drive), self-elimination leaves no transgene sequence behind and thus nothing to reconstitute. For Ae. aegypti, many of the transgenic strains that are required for these experiments have been developed by the inventors, including maternal/zygotic expression of the Tta transactivator, the kmo^{EGFP} recipient strain, and transgenic strains that validate the HSP70 promoter.

### Example 28: Evaluation of Self-Elimination Mechanism on the Spread and Persistence of a Gene Drive Transgene in a Randomly Mating Population

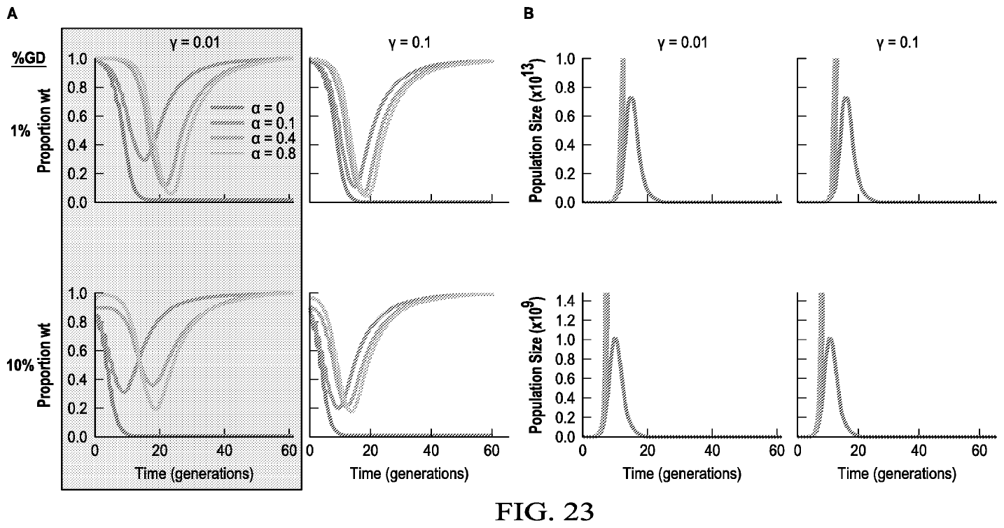

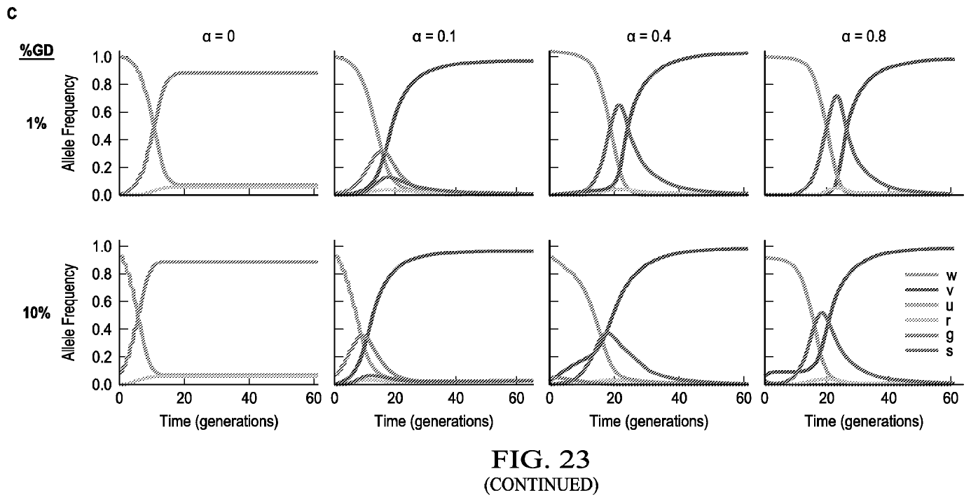

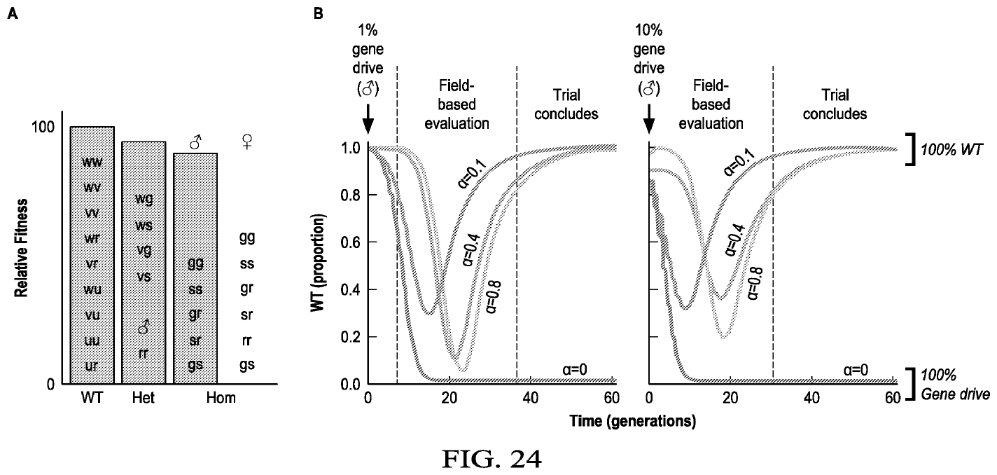

To evaluate how such a self-elimination mechanism might affect the spread and persistence of a gene drive transgene in a randomly mating population, previously developed deterministic models for homing-based gene drive were modified to incorporate a probability for both successful and failed transgene elimination. In total, the model considered six allele types (w, v, g, s, u, and r; FIG. 19A) and six rates of self-elimination mechanisms (FIG. 19B) that govern how each of the alleles can be generated or lost. Previous models and accumulating biological data agree that when the fitness cost associated with disruption of a host gene by a gene drive transgene is small (FIG. 20), resistance alleles arise and displace the invading gene drive. For a gene drive targeted to a non-essential gene, the addition of a self-elimination mechanism acting at just 10% efficiency is predicted to dramatically accelerate the displacement of gene drive alleles (FIG. 20), which could be slowed, but not prevented, by increasing the self-elimination mechanism failure rate (FIG. 21).

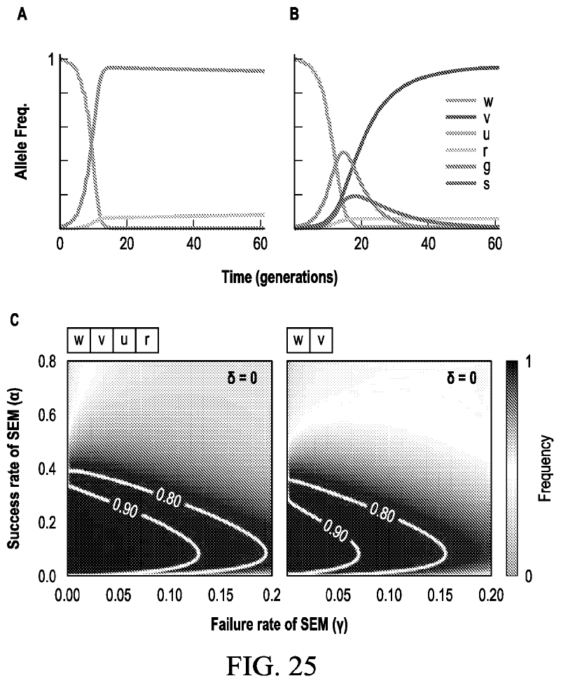

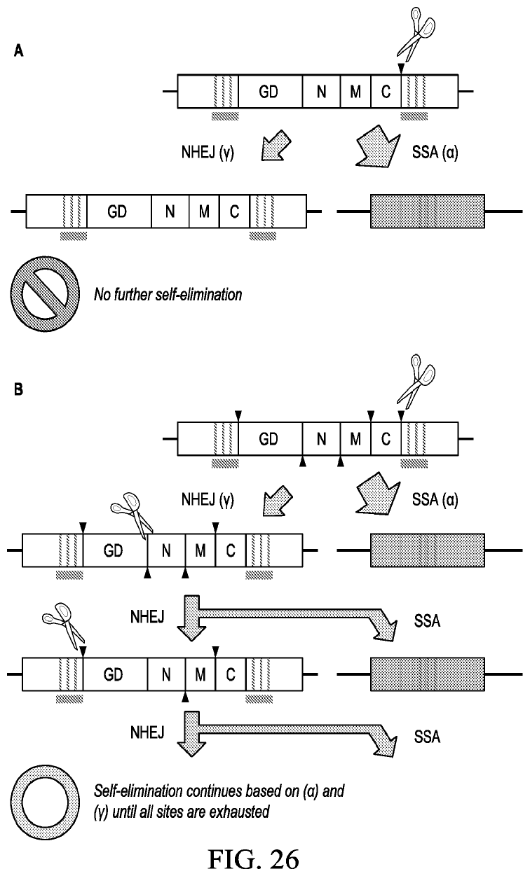

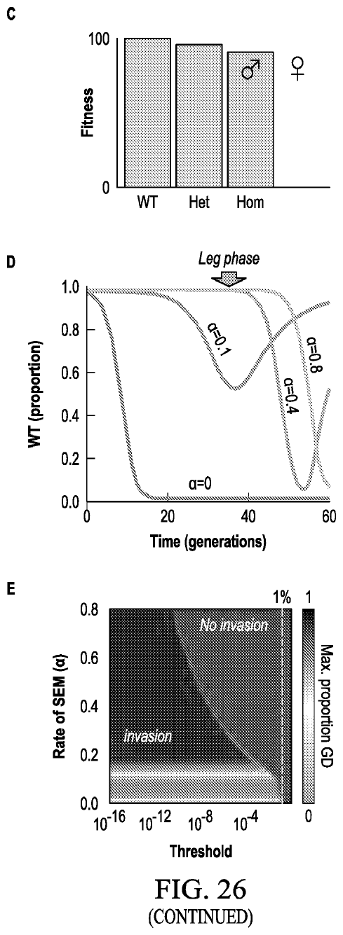

As the probability of generating low-cost resistance alleles decreases, the expected persistence of a gene drive transgene in a population is expected to increase (FIG. 22). However, the incorporation of a self-elimination mechanism prevented the fixation of such a strong gene drive transgene and rapidly restored wild-type genotypes across a wide range of efficiencies (10-80%, FIG. 24). Dsx-like gene drive transgenes were removed from the population even at a self-elimination mechanism breakdown rate of 10%; this was sufficient to averted complete population collapse (FIG. 23). Importantly, the inclusion of an active self-elimination mechanism did not prevent the initial invasion of the target population by the gene drive transgene, but rapidly reversed its prevalence (temporal control).

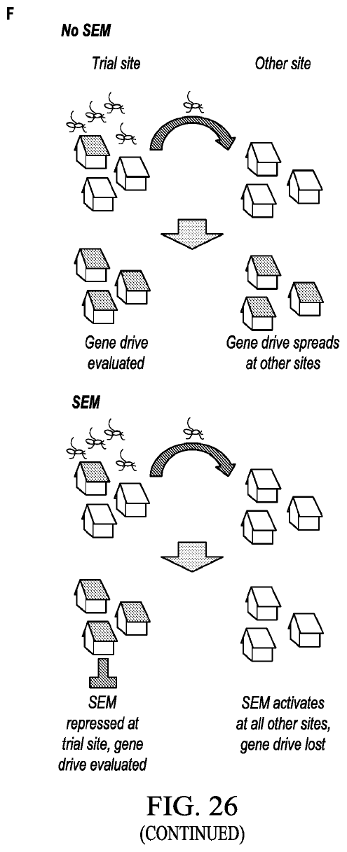

To better understand the underlying dynamics, individual allele frequencies were calculated in the absence (FIG. 25A) or presence (FIG. 25B) of a self-elimination mechanism when naturally occurring resistance alleles cannot be selected for due to their high fitness costs. Without the self-elimination mechanism, gene drive alleles rapidly dominate the population, with a small percentage of high-cost resistance alleles making up a consistent low-level minority. In contrast, no-cost resistance alleles (v) generated by the self-elimination mechanism quickly overtook gene drive alleles, which were lost from the population (FIG. 25B, FIG. 23C). This was true for a broad range of rates for both self-elimination mechanism (0-80%) and self-elimination mechanism failure (0-20%), despite the absence of selection for natural resistance alleles (δ=0), as the inclusion of a self-elimination mechanism led to the restoration of the population to a transgene-free status FIG. 25C, FIG. 23C). Incorporating a self-elimination mechanism approach into a homing-based gene drive transgene can potentially provide unprecedented control over the persistence of these invasive genetic elements while still allowing their temporary spread into a target population during field-based evaluation and risk assessment.

### Example 29: Evaluation of Effect of Multiplexing Self-Elimination Mechanism

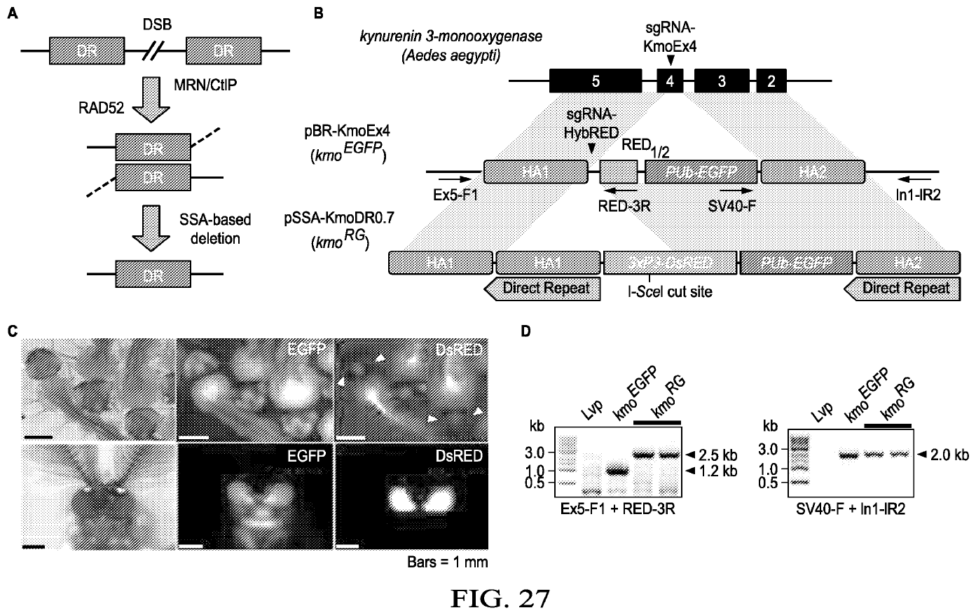

The potential for multiplexing to increase self-elimination efficiency and prevent gene drive invasion into sites outside of any potential trial area (spatial control) was next evaluated, as currently proposed methods for spatial control of gene drive require multiple independently segregating transgenes, bioremediation, or both. In one embodiment, self-elimination mechanisms based on a nuclease-induced double-stranded DNA break and SSA repair (FIG. 26A) could be multiplexed by simply increasing the number of nuclease recognition sites in the gene drive transgene (FIG. 26B).

A gene drive scenario based on the disruption of a gene critical for female fertility such as dsx (FIG. 26C) was modelled, and this time allowed five independent attempts at transgene elimination. Multiplexing of the self-elimination mechanism substantially delayed, but never prevented, invasion of the gene drive transgene in the simulated population (FIG. 26D). This model only allows allele frequencies to approach, but never actually reach zero, it has been considered that during the extended lag phase observed for even moderate values of self-elimination mechanism (0.4), the allele frequency of the gene drive transgene might fall so close to zero as to be considered practically zero. The maximum frequency was plotted of the gene drive transgene at any point during the simulation for arbitrary thresholds (not to be confused with the threshold for invasion of the gene drive transgene itself) down to 10-16, below each of which it was considered lost due to a stochastic event (FIG. 26E). While a relatively crude method of introducing stochasticity, the inclusion of a multiplexed self-elimination mechanism reduced the frequency of the dsx gene drive transgene in the target population by up to 6-7 orders of magnitude below the initial release frequency. Altogether, these data suggest that at high rates (>0.8), a multiplexed self-elimination mechanism may serve as a form of biocontainment for low-threshold gene drives (spatial control, FIG. 26F), while at lower rates (>0-0.2) even a single self-elimination mechanism renders the gene drive essentially biodegradable (temporal control).

### Example 30: Model Structure and Equation Generation

For each of the gene drive mechanisms, a system of delayed differential equations was developed that predicted the number of offspring generated during each time step. Malthusian population growth was assumed with a daily time step through the models. Differential equations were concatenated and analysed using MATLAB 2017b. A single core with 8 GB of memory was sufficient for running MATLAB models to capture the proportions of wild-type individuals and allele progressions for all models. Parameter spaces for the remaining models utilized 112 cores with 392 GB of memory for up to 24 hours from the Texas A&M University High Performance Research Computing (HPRC) Terra cluster for the computation of these parameter spaces. Model outputs were saved to a comma-separated values (.csv) file and plotted using Python 3.7.

The system dynamics models returned the number of adult and juvenile individuals of each genotype for every time step throughout the simulation. Initial model parameters are provided in Table 4.

TABLE 4
Variable Definitions

| Variable | Description | Value |
|---|---|---|
| λ | Female reproduction rate (per day) | 7 |
| σ | Proportion of female offspring | 0.5 |
| cᵢ | Fitness cost of genotype i | Varies |
| μ_{A} | Adult mortality rate (per day) | 0.3 |
| μⱼ | Juvenile mortality rate (per day) | 0.03 |
| η | Development time (in days) | 12 |

Using the fitness costs (c) associated with each genotype and sex (i), adult and juvenile mortality rates (μ_{A} and μ_{J}, respectively) were adjusted such that the mortality rate could not be more than 1, giving:

$${μ}_{{A}_{i}} = \frac{{μ}_{A}}{( 1 - {c}_{i} )} for ( 1 - {c}_{i} ) \geq {μ}_{A} , otherwise {μ}_{Ai} = 1 \\ {μ}_{{J}_{i}} = \frac{{μ}_{J}}{( 1 - {c}_{i} )} for ( 1 - {c}_{i} ) \geq {μ}_{J} , otherwise {μ}_{Ji} = 1$$

Mortality rates were applied at each time step, where the surviving number adult individuals of each genotype Aᵢ(T) was calculated by reducing the number of adult individuals of each genotype at the previous time step Aᵢ(T−1) by the mortality rate, such that:

Aᵢ(T)=(1−μ_{A}_{ᵢ})Aᵢ(T−1)

Juvenile mortality was applied at the time the juveniles became adults, where the number of juvenile individuals surviving the development period (η) was defined as:

Jᵢ(T−η)(1−μ_{J}_{ᵢ})^{η}

Combining the surviving adults with the fully developed juveniles (also now adults), the number of adults with a particular genotype at time T can be defined as the number of adults surviving a single time increment (from time T−1) and the number of surviving juveniles (from time T−η), such that:

Aᵢ(T)=(1−μ_{A}_{ᵢ})Aᵢ(T−1)+Jᵢ(T−η)(1−μ_{J}_{ᵢ})^{η}

The number of females with a particular genotype Fᵢ was directly used in calculating the number of offspring produced. Since males do not directly produce offspring, the proportion of adult males with a particular genotype Mᵢ was calculated such that:

$${M}_{i} = {A}_{{M}_{i}} {\sum }_{i = 1}^{n} \frac{1}{{A}_{{M}_{i}}}$$

Utilizing the equations generated for the calculation of the number of offspring of each genotype, the fitness costs, initial input, self-elimination (α, β, γ), the probability of double-stranded break induction (q, 0.95) and the probability of homology-dependent repair (p, 0.95), the number of offspring created for each time step were calculated.

For equation generation a two-dimensional matrix was generated of all the possible genotypes of females (Fᵢ) and males (Mᵢ). A third dimension was added to capture every possible outcome of offspring (gᵢ). The value of each index within this three-dimensional matrix corresponded to the probability that the combination of the two parental genotypes would produce the respective offspring of the genotype. Iterating through all possible combinations of Fᵢ, Mᵢ, and gᵢ, a matrix of probabilities was generated. Once the matrix was fully populated, a string was concatenated with the parental genotypes and probability of producing an offspring, resulting in the form:

Fᵢ*Ψ(gᵢ|Fₗ,Mₗ)*Mₗ

This was utilized in the calculation of the number of offspring in the system dynamics model. All combinations of parental genotypes to create a particular offspring genotype k were concatenated in the form:

$${g}_{i} = {\sum }_{j = 1}^{l} {\sum }_{k = 1}^{n} {F}_{j} * Ψ ( {g}_{i} \vert {F}_{j} , {M}_{k} ) * {M}_{k}$$

Equations were simplified using MATLAB's str2sym function to reduce the additional computations necessary when referencing and calculating equations from the system dynamics model. To calculate the daily number of offspring of genotype i that were being produced, daily reproduction rates, sex ratio, and fitness costs were additionally concatenated into the equation following the simplification of the equations, for females giving:

$$\frac{\partial {g}_{i}}{\partial t} = λ * σ * ( 1 - {c}_{i} ) {\sum }_{j = 1}^{l} {\sum }_{k = 1}^{n} [ {F}_{j} * Ψ ( {g}_{i} \vert {F}_{j} , {M}_{k} ) * {M}_{k} ]$$

and for males giving:

$$\frac{\partial {g}_{i}}{\partial t} = λ * ( 1 - σ ) * ( 1 - {c}_{i} ) {\sum }_{j = 1}^{l} {\sum }_{k = 1}^{n} [ {F}_{j} * Ψ ( {g}_{i} \vert {F}_{j} , {M}_{k} ) * {M}_{k} ]$$

### Example 31: Establishment of an SSA-Based Transgene Removal System in Aedes aegypti

To control vector mosquito populations, genetics-based control methods have been proposed based on Sterile Insect Technique (SIT), Release of Insects carrying a Dominant Lethal (RIDL) and/or gene drive. In gene drive approaches, the modified organism carries one or more genetic elements that permits the rapid introgression of the genetic trait into the target species population via super-Mendelian inheritance. The development of the Clustered Regularly Interspaced Short Palindromic Repeats/CRISPR-associated protein 9 (CRISPR) system dramatically accelerated homing gene drive strategies in malaria or dengue transmitting mosquitoes. CRISPR-based homing gene drive approaches have been proposed that could permanently alter the genomes of disease vectors for the purposes of either population suppression or population replacement (rendering vectors unable to transmit pathogens). Meanwhile, concerns have been raised that gene drive transgenes could potentially invade non-target populations, and given their invasive nature it may be impossible to remove such transgenic material once out in the field, while potential hazards to ecosystems are still uncertain. The use of split-drives or other mitigation approaches have been proposed to make gene drive both confinable and potentially reversible. While these approaches could limit the process of gene drive, removing the transgenes themselves is not simple and in many cases would require remediation in the form of mass release of wild-type insects.

Mosquitoes, like all eukaryotes, rely on DNA repair systems to process DNA double-strand breaks (DSBs) by mainly two pathways; non-homologous end joining (NHEJ) or homology-directed recombination (HDR). In NHEJ, the Ku complex initially binds the DSB site and subsequently recruits the DNA-PKcs/Artemis complex and the XRCC4-DNA Ligase IV complex to repair the broken DNA ends, potentially generating insertions or deletions in the process. In contrast, the HDR pathway can repair DSBs by using a homologous template sequence from a sister chromosome. In the latter case, DNA end-resection at the DSB site results in a 3′ single-stranded DNA (ssDNA) tail that allows other necessary factors including the MRN/X complex, RAD51, and BRCAs to be recruited for strand invasion during the repair process.

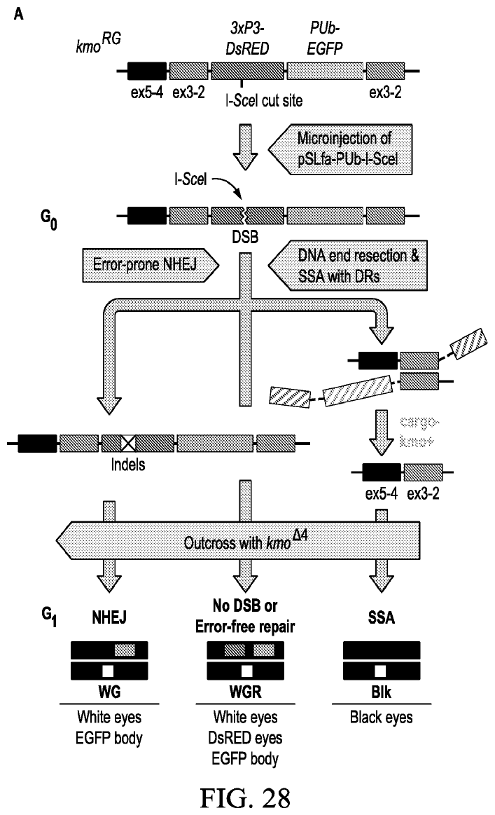

Interestingly, when the DSB-induced ssDNA resection occurs between two parallelly identical sequences, known as direct repeats (DRs), the single-strand annealing (SSA) pathway allows the DRs to be annealed and triggers the intervening sequences to be deleted (FIG. 27; Panel A).

The example describes an exemplary system to pre-program the elimination of transgene cargos in the mosquito Aedes aegypti. Site-specific recombination was used to insert two transgenes within the Ae. aegypti kmo locus. DSB induction triggered SSA-based repair, removing all exogenous cargo and flawlessly restoring the wild-type gene and the normal eye pigmentation phenotype from the transgenic white-eyed mosquitoes. Moreover, multigenerational tests indicate that the rate of SSA-based transgene elimination assisted by natural selection substantially increased the number of wild-type individuals in the test populations. In certain embodiments, the SSA-based biodegradable transgene system described herein exemplifies a rescue strategy for transgenesis-based mosquito population control. For instance, an SSA-based rescuer strain (kmoRG) was engineered to have direct repeat sequences (DRs) in the Ae. aegypti kynurenine 3-monooxygenase (kmo) gene flanking the intervening transgenic cargo genes, DsRED and EGFP. Targeted induction of DNA double-strand breaks (DSBs) in the DsRED transgene successfully triggered complete elimination of the entire cargo from the kmo^{RG} strain, restoring the wild-type kmo gene and thereby normal eye pigmentation.

In this example, the Aedes aegypti Liverpool wild-type strain (Lip), the TALEN-generated kmo-null mutant strain (kmo^{Δ4}) (Aryan et al., PLoS ONE 8 (2013)), and all transgenic strains were maintained at 27° C. and 70% (±10%) relative humidity, with a day/night cycle of 14 hours light and 10 hours dark. Larvae were fed on ground dry fish food (Tetra), and adult mosquitoes were fed on 10% sucrose solution. The mated females were fed on defibrinated sheep blood (Colorado Serum Company) using the artificial membrane feeder.

To generate pSSA-KmoDR0.7, the donor DNA for kmo^{RG}, three plasmids (pGSP1-KmoHA1-DR0.7, pGSP2.3-DsRED-SV40, and pGSP3.8C-EGFP-KmoHA2) were modified from the synthesized plasmid templates (GenScript) and assembled by Golden Gate Assembly (NEB). pGSP1-KmoHA1-DR0.7 contained kmo exon4/5 (homology arm 1 [HA1]) and kmo exon2/3 (homology arm 2 [HA2], direct repeat [DR]). pGSP2.3-DsRED-SV40 encoded 3×P3-DsRED-SV40, in which the homing endonuclease I-SceI recognition site (5′-TAGGGATAACAGGGTAAT-3′) (SEQ ID NO: 1) was engineered in-frame next to ATG translation start codon of DsRED. pGSP3.8C-EGFP-KmoHA2 included the PUb-EGFP-SV40 and kmo exon2/3 (HA2, DR). For the donor DNA for kmo^{EGFP}, Golden Gate Assembly using pGSP1-KmoHA1, pGSP2-REDh-SV40, and pGSP3.8C-EGFP-KmoHA2 generated pBR-KmoEx4. pGSP1-KmoHA1 was made by replacing the kmo exons 2-to-5 sequence in pGSP1-KmoHA1-DR0.7 with the KpnI-AgeI fragment of kmo exon4/5. pGSP2-REDh-SV40 was modified from pGSP2.3-DsRED-SV40 by removing the AscI and SbfI fragment of the 3×P3 promoter and the 5′-half of DsRED containing the I-SceI site. Sequential blunting and ligation of both enzyme-cut ends (AscI and SbfI) created the sgRNA-HybRED site that is unique to REDh.

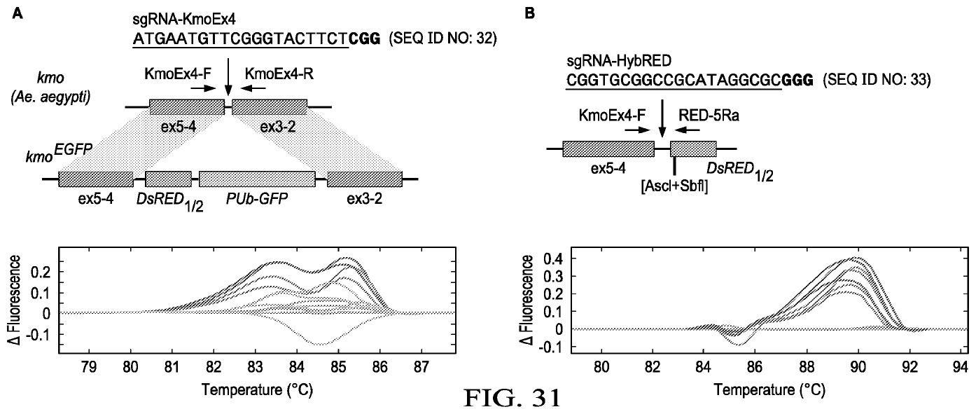

To establish an SSA-based transgene removal system in Aedes aegypti, site-specific insertion of transgene sequences targeting the kynurenine 3-monooxygenase (kmo) gene as the recipient locus in a two-stage process (FIG. 27; Panel B and Table 5) was performed. For the 1^{st} stage, a polyubiquitin-EGFP (PUb-EGFP) reporter cassette and the 3′ portion of the DsRED (RED_{1/2}) gene were flanked by homology arm (HA) sequences (771 bp from exon4/5 for HA′ and 684 bp from exon2/3 for HA2) with DSB induction triggered by Cas9 complexed with a single synthetic guide RNA (sgRNA-KmoEx4; FIG. 31; Panel A and Table 6). EGFP⁺ individuals were used to establish a strain referred to as kmo^{EGFP}. In the second stage, a new sgRNA (sgRNA-HybRED) was designed to recognize the boundary sequence of the RED_{1/2} in the kmo^{EGFP} strain (FIG. 31; Panel B), with the new transgene sequences flanked by corresponding homology arms (FIG. 27; Panel B). More specifically, site-specific integrations at the Ae. aegypti kmo site were obtained by microinjection into pre-blastoderm embryos as previously described (Aryan et al., Methods 69 (2014); Kistler et al., Cell Reports 11 (2015); Basu et al., Methods in Molecular Biology, (2016)). For the kmo^{EGFP} strain, the injection mix included 0.4 μg/μl of CRISPR/Cas9 enzyme (PNA Bio), 0.1 μg/μ1 of sgRNA-KmoEx4, and 0.3 μg/μl of donor plasmid pBR-KmoEx4 was microinjected to the Lvp wild-type embryos. The G₂ kmo^{EGFP} strain was utilized as a recipient for a second round of microinjections using sgRNA-HybRED, Cas9, and pSSA-KmoDR0.7 (same concentrations as above) to generate the kmo^{RG} strain. Chromosomal integration of the transgenes at the kmo locus was confirmed by PCR analysis using genomic DNAs purified from a single G₂ individual larva as the template and a primer set that is specific to the transgene or kmo (FIG. 27; Panel B and Table 6). PCR was performed using the Phusion High-Fidelity DNA polymerase (NEB) for 35 cycles: 95° C. for 30 sec, 58° C. for 30 sec, and 72° C. for 2 min.

The result of this integration was that the HA2 region was duplicated next to HAL creating direct repeats (DRs) of approximately 700 bp that could be utilized by the SSA pathway. This two-stage process prevented competition in repair between the two HA2 motifs, as use of the HA2 in proximity to HA1 could result in repair of the kmo gene with no integration of the transgenes. As expected, the stage 2 kmo^{RG} mosquitoes displayed DsRED fluorescence in the eyes, EGFP fluorescence in the body, and white-colored eyes due to loss of kmo (FIG. 27; Panel C). The site-specific insertion of each cassette was verified by PCR analysis for both kmo^{EGFR} and kmo^{RG} strains (FIG. 27; Panel D). In order to trigger a DSB in the transgene sequence and initiate SSA, an I-SceI recognition site was included in-frame following the ATG translational start codon of the DsRED gene. This position was advantageous in that it could potentially allow the identification of NHEJ-based repair events (DsRED⁻, EGFP⁺, white eye) in addition to SSA-based events (DsRED⁻, EGFP⁻, black eye).

TABLE 5
Generation of CRISPR/Cas9-driven transgenic lines.

| Transgenic | Donor | | Recipient | # Embryos | # G₀ Larvae | | | # G₁ Larvae w/phenotypes^{b} | | | |
|---|---|---|---|---|---|---|---|---|---|---|---|
| mosquitos | DNAs | sgRNAs | strains | injected | survived | # G₀ outcrossed^{a} | | Blk | BlkG | WG | WGR |
| kmo^{EGFP} | pBR- | KmoEx4 | Lvp | ~2,000 | 125 | ♂51 | ×Lvp | 12,045 | 78 | | |
| | KmoEx4 | | | | (6.25%) | ♀60 | | | (0.64%) | | |
| kmo^{RG} | pSSA- | HybRED | kmo^{EGFP} | ~2,080 | 258 | ♂109 | ×kmo^{Δ4} | | | 14,949 | 32 |
| | KmoDR | | | | (12.4%) | ♀126 | | | | | (0.2%) |

^{a}kmo^{Δ4} is the TALEN-generated kmo -null mutant strain (29).
^{b}Marker phenotypes: W, white eyes; Blk, black eyes; G, EGFP; R, DsRED.

TABLE 6
List of oligonucleotides for SgRNAS, PCR, and subcloning.

| Oligonucleotdes | Sequences (5′ to 3′)^{a} |
|---|---|
| sgRNA-KmoEx4 | GAAATTAATACGACTCACTATAGGATGAATGTTCGGGTACTTCTGTTTTAGAGC |
| | TAGAAA (SEQ ID NO: 2) |
| sgRNA-HybRED | GAAATTAATACGACTCACTATAGGCGGTGCGGCCGCATAGGCGCGTTTTAGAGC |
| | TAGAAA (SEQ ID NO: 3) |
| KmoEx4-F | TGTGAGTAGRTTCCTTCGTCGTTGG (SEQ ID NO: 4) |
| KmoEx4-R | ATTCCGTAGCAAGTTTACCTTGGGC (SEQID NO: 5) |
| DmHsp70-F | AGCAAAGTGAACACGTCGCTAAGCG (SEQ ID NO: 6) |
| DsRED-5Ra | TCACCTTCAGCTTCACGGTCTTGTCC (SEQ ID NO: 7) |
| KMF2 | TTCTTCAAGACCAGGCCTCAATC (SEQ ID NO: 8) |
| KMR1 | TCACTAAACTCAGCCAGTATCCTAT (SEQ ID NO: 9) |
| Ex5-F1 | ACGACCGCATACAAAACGTACG (SEQ ID NO: 10) |
| RED-3R | TCGTACTGCTCCACGATGG (SEQ ID NO: 11) |
| SV40-F | AATCAGCCATACCACATTTGTAGAGG (SEQ ID NO: 12) |
| In1-IR2 | AATCATGGGTAGGACGAATGTCTTAGTCAGG (SEQ ID NO: 13) |
| KmoHA1-F-Kpn | TTTTGGTACCGCCAGATCGCAGATAGAGTGTGC (SEQ ID NO: 14) |
| KmoHA1-R-Age | TTTTACCGGTACCCGAACATTCATCTTTATTTC (SEQ ID NO: 15) |
| KmoHA2-F-Av2 | TTTTCCTAGGCGGCCGCTAAAATAAACAACATTATCAG (SEQ ID NO: 16) |
| KmoHA2-R-Av2 | TTTTCCTAGGGTTGGCTCTCTATTTGCACTCCACC (SEQ ID NO: 17) |
| NosPro-F-M | GTCAACGCGTGGATCACTATCAAACCCCTAAGGAC (SEQ ID NO: 18) |
| NosPro-R-B | GTCAGGATCCAGACATCCTCTAGATTTGTTCGTTGATC (SEQ ID NO: 19) |
| Scel-F-B | GTCAGGATCCATGCCCAAGAAGAAGCGCAAGG (SEQ ID NO: 20) |
| Scel-R-S | GTCAGTCGACTTATTTCAGGAAAGTTTCGGAGGAG (SEQ ID NO: 21) |
| Nos3UTR-F-N | GTCAGCGGCCGCTCTAGACGTAATCGAAGTGTTGGAC (SEQ ID NO: 22) |
| Nos3UTR-R-XR | GTCAGAATTCCTCGAGCGCCCTTTTCGTCATAAAATCGTAG (SEQ ID NO: |
| | 23) |
| MRF1 | AAGACGATGAGTTCTACTGGCGTGGAATCC (SEQ ID NO: 24) |
| MRR1 | CTTGCCGTATGTGATGCAGCGTTGTCATGG (SEQ ID NO: 25) |
| MLF1 | TTGTTTACTCTCAGTGCAGTCAACATGTCG (SEQ ID NO: 26) |
| MLR1 | TTCGACAGTCAAGGTTGACACTTCACAAGG (SEQ ID NO: 27) |

^{a}sgRNA target sequences are shown in bold letters. Restriction enzyme site sequences were underlined

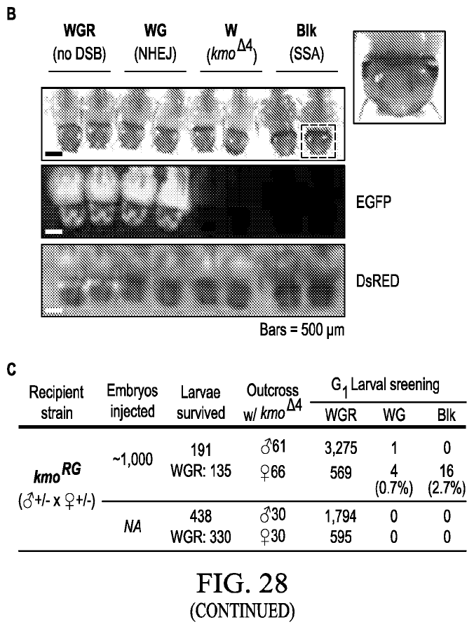

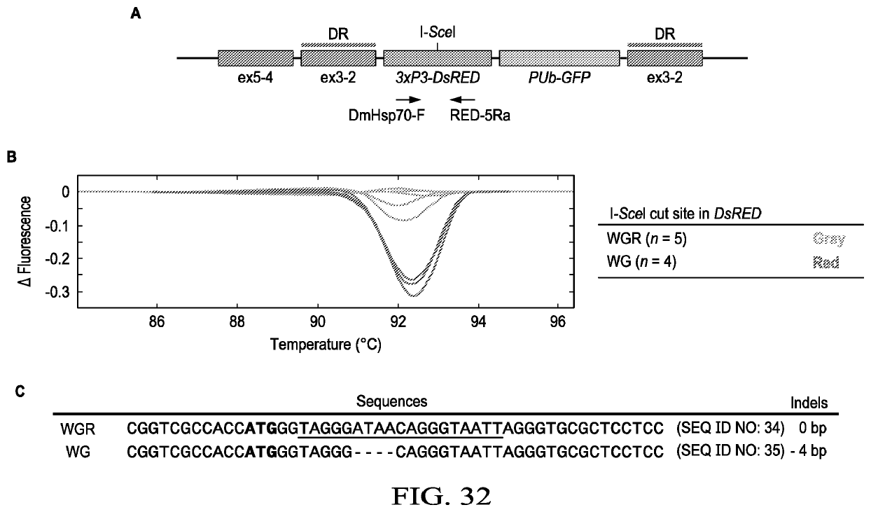

As an initial test of SSA-driven elimination of the transgene in the kmo^{RG} strain, pre-blastoderm embryos were microinjected with a donor plasmid expressing the homing endonuclease (HE) I-SceI to induce DSB formation in the transgene (FIG. 28; Panel A). G₀ survivors of the injection procedure were crossed with kmo^{Δ4}, a white-eyed non-transgenic strain with a characterized disruption in kmo, with G₁ progeny scored for both fluorescent markers and eye pigmentation to determine the rates of DNA repair proceeding through either the NHEJ or SSA pathways. Consistent with SSA-driven elimination of the transgenes, ˜2.7% of the progeny of female G₀ survivors were restored to black eyes (FIG. 28; Panel B and C). In contrast, the NHEJ-driven loss of the DsRED marker alone was observed in just 0.7% of female progeny. No SSA-based events were recovered from male progeny, potentially due to the inability of the injected HE donor plasmid to be inherited through the male germline. It was confirmed that the loss of DsRED in two G₀♀-G₁ mosquitoes identified as DsRED⁻/EGFP⁺/white-eye (WG) was indeed due to imprecise repair at the I-SceI target site resulting in a 4 bp deletion (FIG. 32). Therefore, SSA-based repair mechanisms can be at least as efficient as NHEJ, if not more so, and can trigger the complete elimination of transgene sequences.

### Generation of Transgenic Strains Expressing Nucleases in Aedes aegypti

Mos1-based plasmid constructs were assembled with 1-SceI under the control of several promoters known to function in Aedes aegypti; nos (Adelman, et al, Proceedings of the National Academy of Sciences of the United States of America 104 (2007); Calvo, et al., Insect Biochemistry and Molecular Biology, (2005)), β2-tublin (Smith et al., Insect Molecular Biology 16 (2007)), PUb (Anderson et al., Insect Molecular Biology 19 (2010); Carpenetti et al., Insect Molecular Biology 21 (2012)), and Hsp70A (Anderson et al., Insect Molecular Biology 19 (2010); Carpenetti et al., Insect Molecular Biology 21 (2012)). Two steps were taken for assembling the donor plasmid constructs. First, the MluI-BamHI fragment of nos (˜1.56 kb) or β2-tublin (˜1.0 kb) promoter, the BamHI-SalI fragment of the I-SceI coding region (˜0.85 kb), and the NotI-EcoRI fragment of nos (˜0.5 kb) or β2-tublin (˜0.2 kb) 3′UTR were obtained by PCR amplifications using primer sets providing the corresponding enzyme sites (Table 6) and sequentially assembled into a universal insect plasmid backbone pSLfa-PUb-mcs (Addgene #52908) to generate pSLfa-Nos-I-SceI or pSLfa-β2T-I-SceI. For pSLfa-PUb-I-SceI, the I-SceI coding sequence was ligated to BamHI and SalI sites in pSLfa-PUb-mcs. For pSLfa-Hsp70A-I-SceI, the MluI-NcoI fragment of Hsp70A promoter (˜1.5 kb) was replaced for PUb promoter (˜1.4 kb) in pSLfa-PUb-I-SceI. Second, the whole DNA piece of Promoter-I-SceI-3′UTR was taken out from the individual pSLfa-based plasmid construct and inserted to MluI and EcoRI sites in pM2-3×P3-BFP, a Mariner Mos1-based plasmid backbone.

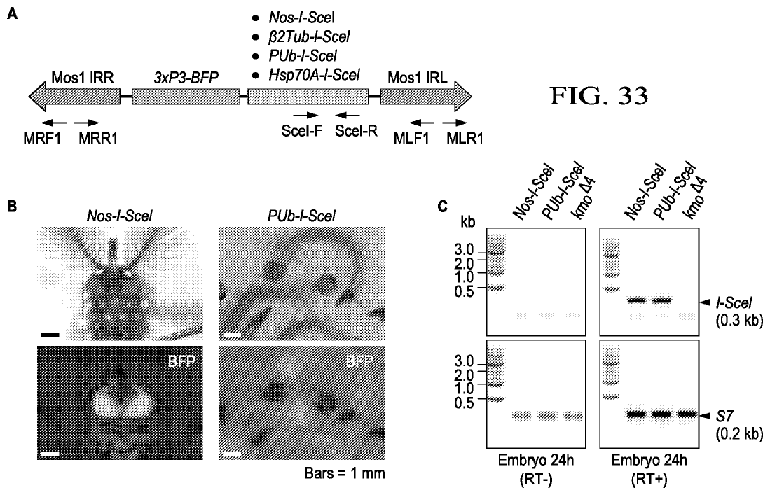

To generate transgenic strains expressing I-SceI, each donor plasmid (0.5 μg/μl), pMOS-3×P3-BFP-Nos-I-SceI, pMOS-3×P3-BFP-32T-I-SceI, pMOS-3×P3-BFP-PUb-I-SceI, or pMOS-3×P3-BFP-Hsp70A-I-SceI, was microinjected into pre-blastoderm embryos of the kmo^{m} strain (Aryan et al., PLoS ONE 8 (2013)), along with the Mos1 helper plasmid (0.2 μg/μl), pKhsp82M (Coates et al., Molecular and General Genetics 253 (1997)). For BFP-positive transgenic mosquitoes, transposon-chromosome junction sequences were identified by inverse PCR using Sau3AI-digested genomic DNA and primers indicated in FIG. 33; Panel A and Table 6. For the evaluation of I-SceI transcripts, total RNA was extracted from 200 embryos at 24 hours after oviposition using the Trizol reagent (Invitrogen). First-strand cDNAs were synthesized from 1 μg of total RNAs using the SuperScript IV VILO Reverse Transcription Kit (Life Technologies). To amplify the transcript-derived cDNA of I-SceI, PCR was performed using the Q5 High-Fidelity DNA polymerase (NEB) and I-SceI gene-specific primers (Table 6) with 35 cycles; 95° C. for 30 sec, 60° C. for 30 sec, and 72° C. for 1 min.

The timing, level and tissue specificity of I-SceI expression is variable when introduced transiently through plasmid injection. Therefore, transgenic strains that express I-SceI under the activity of germline-specific nos and beta2-tubulin (β2T), whole-body constitutive polyubiquitin (PUb), or heat-inducible heat shock protein 70A (Hsp70A) promoters (FIG. 33; Panel A) were sought to be generated as detailed above. Following microinjection to kmo^{Δ4} embryos, one transgenic mosquito strain each for Nos-I-SceI and PUb-I-SceI (FIG. 33; Panels B and Table 7) was obtained. Both Nos-I-SceI and PUb-I-SceI strains were shown to successfully express I-SceI transcripts in embryos at 24 hr post oviposition by RT-PCR analysis (FIG. 33; Panels C), and transgene integration into the mosquito genome was validated by inverse PCR analysis (Table 8).

TABLE 7
Generation of Mariner Mos1-driven transgenic lines

| Transgenic | Donor | Recipient | # Embryos | # Larvae | # G₀ Adults × | # G₁ Larvae w/phenotypes^{b} | |
|---|---|---|---|---|---|---|---|
| mosquitoes | Plasmids | strains^{a} | injected | survived | kmo^{Δ4} | W | WB |
| Nos-I-SceI | pMOS-3xP3-BFP-Nos- | kmo^{Δ4} | ~1,450 | 183 | ♂87 | 570 | 1 |
| | I-SceI | | | (12.6%) | ♀69 | | (0.18%) |
| PUb-I-SceI | pMOS-3xP3-BFP- | kmo^{Δ4} | ~1,150 | 263 | ♂107 | 1,704 | 96 |
| | PUb-I-SceI | | | (22.9%) | ♀116 | | (5.3%) |
| β2T-I-SceI | pMOS-3xP3-BFP-β2T- | kmo^{Δ4} | ~1,150 | 137 | ♂61 | 2,240 | 0 |
| | I-SceI | | | (11.9%) | ♀60 | | |
| | | | ~1,600 | 240 | ♂128 | 19,253 | 0 |
| | | | | (15%) | ♀98 | | |
| | | | ~1,900 | 207 | ♂103 | 11,869 | 0 |
| | | | | (10.9%) | ♀104 | | |
| Hsp70A-I-SceI | pMOS-3xP3-BFP- | kmo^{Δ4} | ~1,600 | 111 | ♂58 | 10,357 | 0 |
| | Hsp70A-I-SceI | | | (6.9%) | ♀41 | | |
| | | | ~1,750 | 353 | ♂165 | 16,919 | 0 |
| | | | | (20.2%) | ♀188 | | |

^{a}kmo^{Δ4} is the TALEN-generated kmo -null mutant strain (29).
^{b}Marker phenotypes: W, white eyes; B, BFP.

TABLE 8
Inverse PCR analysis reveals chromosomal sequences flanking
the transgene in the Nos-I-scel or Pub-I-Scel strain.

| Mariner Mos1 | | |
|---|---|---|
| transgenic | Contig | |
| lines | Identification No. | Flanking Sequences |
| MOS-3xP3-BFP- | aag2_ctg_260:1:1761520:1 | GATCCAATGCTGAGGAAATTACAAATGTTTTTTCAGTGTGTTTTTTGA |
| Nos-I-Scel | | AAAATGTCACAACTTTAAGTTAAAGTTTAGTATACAAAATTCAAAAAA |
| | | TGTTTTTGGAAAAATACATACGAAGTAAGAATAACTCTTTGCCTTTCG |
| | | AATGCGGCTTAGAGAGTTTCATTAGACGCGTAATCACAGAGATATGGA |
| | | CAGAACACTTTTGTATGTTGTTGAGGGGGTGAACCCAAACTTTTGCAC |
| | | GGGAGTGTA=MosRH-3xP3:BFP-Nos:SceI-MosLH-TATATGGC |
| | | GAATACAGAACGAAACTACATCTTGAAATTTGAAAAGAACAAAATTGT |
| | | GAATCGAACAGCCTTGAATGTTTAGAATTTGATTGGGTACTTATTCGT |
| | | CTGTAGTGCTTTTTATCTCTCTCTCTGCTGATGCATAATTTGAACGCA |
| | | TAACGACTTTCAGACTTCACAAGTTCTAGCAACATTTCAACTTATAAT |
| | | CGTTGAAAAGTATACAACATTAAGATTTCAAAATGATGATC (SEQ |
| | | ID NO: 28 (left ar), SEQ ID NO: 29 (right arm)) |
| MOS-3xP3-BFP- | aag2_ctg_279:1:1681516:1 | GATCCAGAATGAGCAAAGTGTCACTTTTA-MosLH-3xP3:BFP- |
| PUb-I-Scel | | PUb:SceI-MosRH-TACTTGCGGAATAATTGAGCGGAACATTTTTCC |
| | | GTACGGAATAGTGACAGCTCCATTTGATTTGTACAGCAGGCGTTACCA |
| | | ATGTTACGAAATCAGCTCTACTTGTCAACTGGATACAGTTCAAGTAAT |
| | | TTGAACAGCTGAAGTATTTCTTGACCATTACTTGTCCTATCCTTTTGC |
| | | ACAGTACTTACGAAGTGGATACCAACCATTA (SEQ ID NO: 30 |
| | | (left arm), SEQ ID NO: 31 (right arm)) |

### SSA-Driven Transgene Elimination in Aedes aegypti

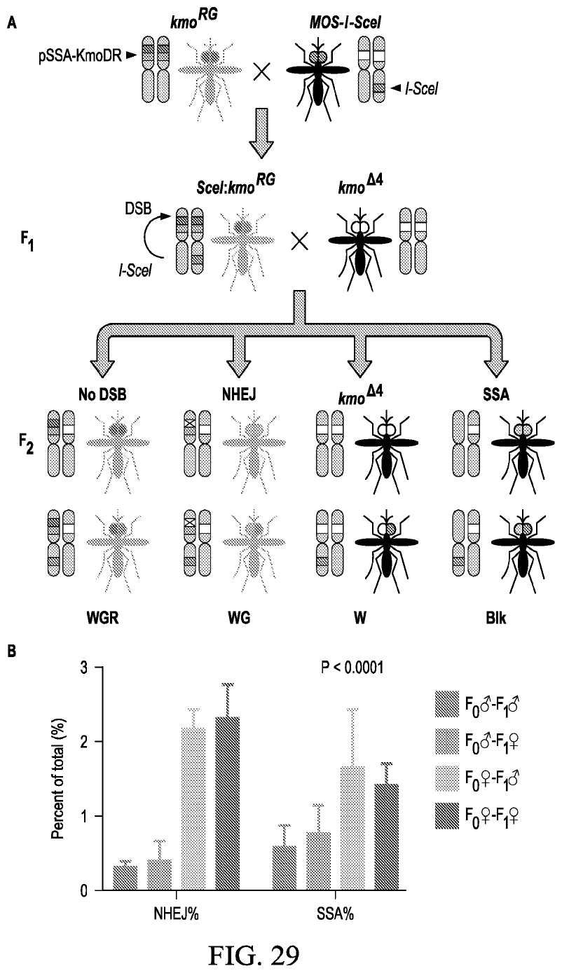

A single-strand annealing (SSA)-based transgene removal system successfully erasing transgenes from the Ae. aegypti genome is described, representing a novel pathway for engineering safety features into approaches for genetic control of vector mosquito populations. To determine the potential for each strain generated to initiate SSA-driven transgene elimination, Nos-I-SceI or PUb-I-SceI mosquitoes were reciprocally crossed with kmo^{RG} (FIG. 29; Panel A). F₁ individuals that contained both sets of transgenes (SceI:kmo^{RG}) were outcrossed to kmo^{Δ4} and F₂ progeny scored for SSA and NHEJ events. More specifically, homozygous kmo^{RG} mosquitoes were reciprocally crossed with the Nos-I-SceI or PUb-I-SceI mosquitoes in a cage of 30 males and 100 females or 20 males and 50 females in triplicate. Fifty male or female F₁ progenies (white-eye, EGFP⁺, DsRED⁺, BFP⁺) were outcrossed with the kmo^{Δ4} strain in a ♂:♀ ratio of 1:3. Female mosquitoes were blood-fed three times, and all subsequent embryos were hatched for F₂ larval screening.

In single-generation SSA tests (Table 9, FIG. 29; Panel B, and Table 10), kmo gene restoration and complete loss of all transgenes in 0.5-1% of transgenic progeny was observed when the grandfather (F₀♂) provided the Nos-I-SceI transgene. Likewise. SSA-based repair events constituted 2-3% of transgenic progeny when the Nos-I-SceI cassette was provided by the grandmother (F₀♀), a potential indication that maternal inheritance increases the absolute number of DSBs induced. Interestingly, though the Nos-I-SceI cassette was not inherited, the F₀♀-F₁ mosquitoes (BFP⁻) were still able to produce DNA repair-associated phenotypes in F₂ progeny (Table 9), providing evidence that significant numbers of DSBs were induced by the dominant maternal effect of the nuclease. In contrast, no NHEJ or SSA events were recovered when using the PUb-I-SceI strain (Table 9), suggesting that expression of I-SceI was insufficient for inducing DSB formation, despite the fact that its transcript was present in embryos (FIG. 33; Panel C). While this result was somewhat unexpected as plasmid-expressed PUb-I-SceI did trigger SSA (FIG. 28; Panel C), the microinjection procedure into pre-blastoderm embryos might have allowed the transiently expressed I-SceI enzyme access to the germ cells, enabling DSB repair events to be transmitted to G₁ progeny, whereas PUb-driven I-SceI gene expression from the chromosome may be restricted in the germline cells, as PUb-driven EGFP mRNA was not detectable in the ovarian tissue. For experiments using a plasmid-based source of I-SceI, 0.5 μg/μl of pSLfa-PUb-SceI (Traver et al., Insect Molecular Biology 18 (2009)) was microinjected into kmo^{RG} recipient embryos obtained from parental self-crossing between heterozygous mosquitoes. Since a mixture of transgenic (75%) and non-transgenic (25%) offspring were expected from this cross, only EGFP⁺/DsRED⁺ survivors were further outcrossed to the kmo^{Δ4} strain. G₁ larvae were scored for either white or black eyes under visible light, and for eye-specific DsRED or whole body EGFP fluorescence using the appropriate excitation/emission filters.

TABLE 9
Single-generation tests for SSA-based transgene elimination induced by
the ISce I-expressing trigger strains (G₄), Nos-I-SceI and Pub-I-SceI.

| | | | F₂ Larval screening^{c} | | | | |
|---|---|---|---|---|---|---|---|
| Parental cross | Lineage of | I-SceI inherited | | # WGR | # WG | # W | # Blk |
| (♂30 × ♀100) | the SSA trigger^{a} | to F₁ adults^{b} | # Total | No DSB | NHEJ | kmo^{Δ4} | SSA |
| Nos-I-SceI × | F₀♂-F₁♂ | + | 7500 | 4122 | 23 | 3315 | 40 |
| kmo^{RG} | | | | | (0.56%) | | (0.97%) |
| | | − | 7252 | 3609 | 1 | 3642 | 0 |
| | | | | | (0.03%) | | |
| | F₀♂-F₁♀ | + | 2588 | 1608 | 9 | 957 | 14 |
| | | | | | (0.56%) | | (0.87%) |
| | | − | 4867 | 2410 | 0 | 2457 | 0 |
| | F₀♀-F₁♂ | + | 7828 | 4268 | 74 | 3380 | 106 |
| | | | | | (1.73%) | | (2.48%) |
| | | − | 7477 | 3839 | 93 | 3528 | 17 |
| | | | | | (2.42%) | | (0.44%) |
| | F₀♀-F₁♀ | + | 4676 | 2722 | 36 | 1835 | 83 |
| | | | | | (1.32%) | | (3.05%) |
| | | − | 5749 | 3077 | 10 | 2652 | 10 |
| | | | | | (0.32%) | | (0.32%) |
| PUb-I-SceI × | F₀♂-F₁♂ | + | 494 | 310 | 0 | 184 | 0 |
| kmo^{RG} | | − | 1211 | 617 | 0 | 594 | 0 |
| | F₀♂-F₁♀ | + | 1370 | 823 | 0 | 547 | 0 |
| | | − | 2904 | 1473 | 0 | 1431 | 0 |
| | F₀♀-F₁♂ | + | 1850 | 1133 | 0 | 717 | 0 |
| | | − | 1302 | 684 | 0 | 618 | 0 |
| | F₀♀-F₁♀ | + | 1450 | 820 | 0 | 630 | 0 |
| | | − | 1277 | 660 | 0 | 617 | 0 |

^{a}nos -driven germline cell-specific expression of the homing endonuclease, I-SceI.
^{b}The Mariner MosI -based transgenic I-SceI allele, which is inherited from the parental SSA trigger strain, provides the eye-specific BFP fluorescence.
^{c}W, white eye; Blk, black eye; G, EGFP; R, DsRED; B, BFP.

TABLE 10
The single-generation test for SSA-based transgene elimination induced by
the ISce I-expressing trigger strains (G₁₂), Nos-I-SceI and Pub-I-SceI

| | | F₂ Larval screening^{b} | | | | |
|---|---|---|---|---|---|---|
| Parental cross | Lineage of | | # WGR | # WG | # W | # Blk |
| (♂20 × ♀50) | SSA trigger (G₁₂)^{a} | # Total | No DSB | NHEJ | kmo^{Δ4} | SSA |
| Nos-SceI × | F₀♂-F₁♂ | 2000 | 990 | 3 | 1002 | 5 |
| kmo^{RG} | | 2080 | 1120 | 3 | 953 | 4 |
| | | 2240 | 1220 | 5 | 1004 | 11 |
| | F₀♂-F₁♀ | 1380 | 700 | 1 | 675 | 4 |
| | | 1635 | 780 | 4 | 847 | 4 |
| | | 1350 | 661 | 4 | 677 | 8 |
| | F₀♀-F₁♂ | 2340 | 1260 | 32 | 1035 | 13 |
| | | 980 | 490 | 10 | 473 | 7 |
| | | 2240 | 1140 | 25 | 1045 | 30 |
| | F₀♀-F₁♀ | 1880 | 950 | 22 | 891 | 17 |
| | | 1240 | 673 | 13 | 546 | 8 |
| | | 2140 | 994 | 29 | 1103 | 14 |
| PUb-SceI × | F₀♂-F₁♂ | 6153 | 3075 | 2 | 3074 | 2 |
| kmo^{RG} | | 6630 | 3480 | 0 | 3150 | 0 |
| | | 9327 | 5170 | 0 | 4157 | 0 |
| | F₀♂-F₁♀ | 1060 | 546 | 0 | 514 | 0 |
| | | 1570 | 954 | 0 | 616 | 0 |
| | | 1610 | 880 | 0 | 730 | 0 |
| | F₀♀-F₁♂ | 269 | 139 | 0 | 130 | 0 |
| | | 1416 | 678 | 0 | 738 | 0 |
| | | 2280 | 1310 | 0 | 970 | 0 |
| | F₀♀-F₁♀ | 2398 | 1300 | 0 | 1098 | 0 |
| | | 2080 | 1100 | 0 | 980 | 0 |
| | | 1783 | 927 | 0 | 856 | 0 |

^{a}nos -driven germline cell-specific expression of the homing endonuclease, I-SceI.
^{b}W, white eye; Blk, black eye; G, EGFP; R, DsRED; B, BFP.

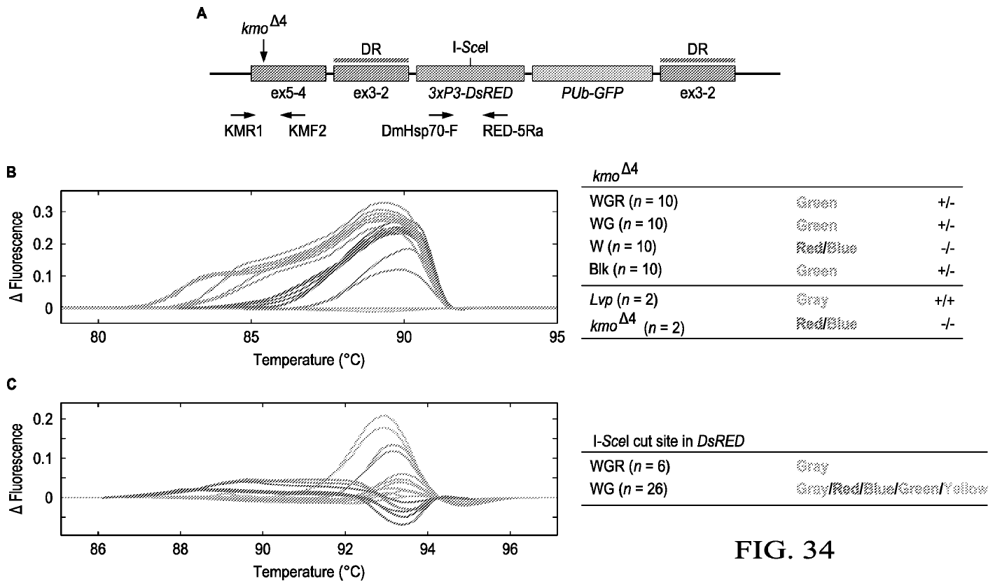

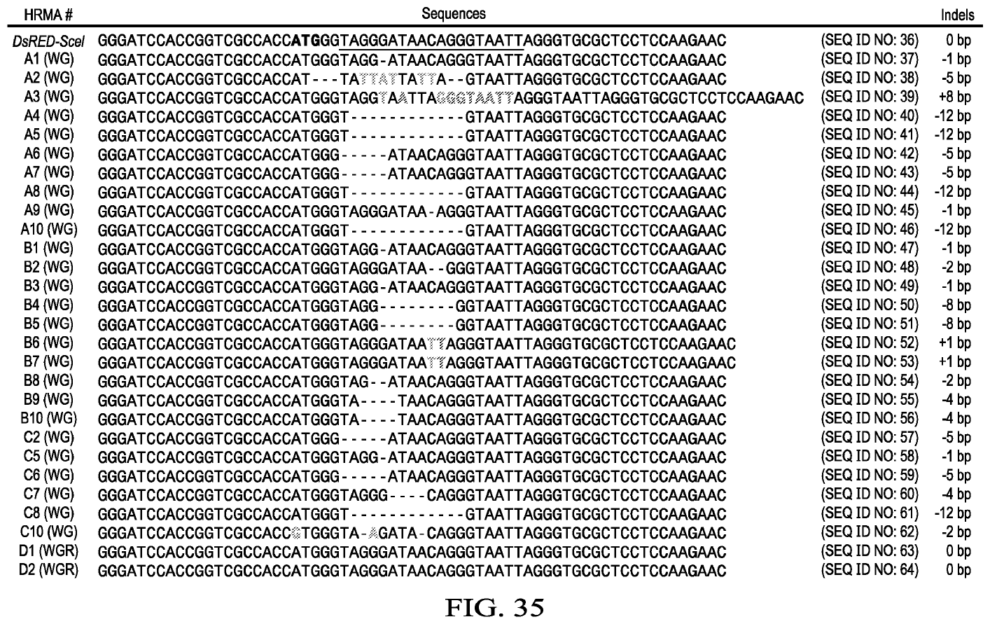

Mosquitoes scored as WG (NHEJ) and Blk (SSA) were confirmed to be heterozygous for the kmo^{Δ4} mutation (FIG. 34; Panel B). In addition, mosquitoes scored as WG were associated with a range of melt-curve profiles (FIG. 34; Panel C), indicative of highly diversified indel mutations caused by the NHEJ pathway. Sequencing analysis of F₂ mosquitoes scored as WG revealed that most indel mutations shifted the DsRED gene out-of-frame (FIG. 35). However, one WG group was shown to have 12 bp in-frame deletion was still scored as phenotypically DsRED-negative. Thus, while missing about ⅓ of NHEJ events was anticipated (in frame deletions that leave DsRED intact), the true number of missed events was likely less than that.

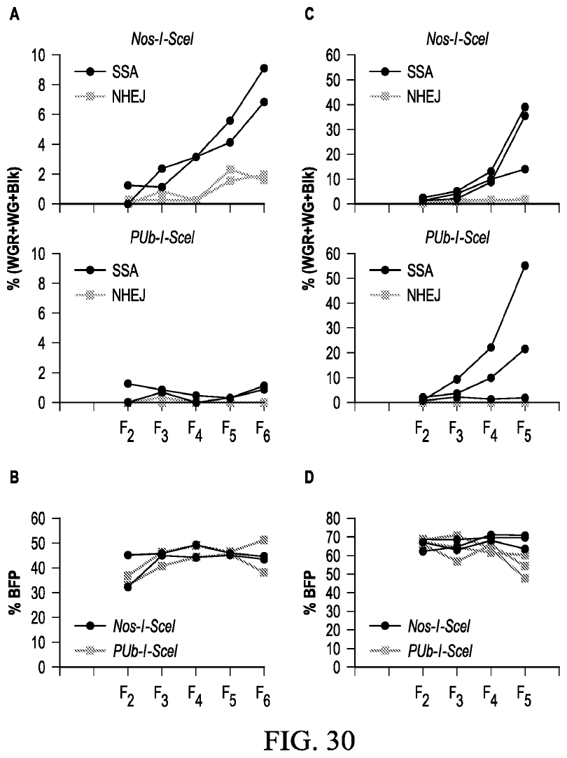

In homing-based gene drive, the conversion of wild-type alleles to transgenics must be a highly efficient process in order to sustain drive. However, basic models suggest even modest SSA efficiencies of 1-3% should be sufficient to restore a population invaded by a homing-based gene drive transgene to a non-transgenic state. For example, kmo^{RG} mosquitoes were allowed to interbreed with Nos-I-SceI or PUb-I-SceI mosquitoes in order to observe if the SSA-based rescue system would be capable of removing transgenes from the kmo^{RG} mosquito population over multiple generations (FIG. 30). To do this, F₁ mosquitoes heterozygous for each transgene (SceI:kmo^{RG}) inherited from F₀ crossing between ♂ Nos-I-SceI or PUb-I-SceI and ♀ kmo^{RG} mosquitoes were self-crossed (Table 9 and Table 10). For each generation starting from F₂, about 1,000 embryos were hatched and all pupae were scored for eye pigmentation and fluorescence to determine DSB repair events (FIG. 30, Table 11. and Table 12), with all individuals placed into a large cage to establish the next generation. The cages were kept in complete darkness for one week to reduce any competitive advantage provided by those individuals with wild-type eye pigmentation during mating. More specifically, thirty Nos-I-SceI or PUb-I-SceI males were crossed with one hundred kmo^{RG} females, to establish each F₀ cage. Only individuals scored positive for all marker phenotypes (white-eye, EGFP⁺, DsRED⁺, BFP⁺) were selected for the F₁ cage of 50 males and 150 females. For each generation, approximately 1,500 embryos were hatched for phenotypic examinations. Male or female pupae were first separated based on eye pigmentation [black-eyed (Blk, kmo⁺) or white-eyed (W, kmo⁻)]. Blk pupae were next screened for EGFP and DsRED fluorescence to identify Blk (kmo⁺, EGFP⁻, DsRED⁻), BlkGR (kmo⁺, EGFP⁺, DsRED⁺) or BlkG (kmo⁻, EGFP⁺, DsRED⁻). The same procedure was repeated for W pupae, which allowed us to identify phenotypic variations of W (kmo⁻, EGFP⁻, DsRED⁻), WGR (kmo⁻, EGFP⁺, DsRED⁺), or WG (kmo⁻, EGFP⁺, DsRED⁻). All groups were then subsequently screened for BFP to track the frequency of the I-SceI transgene in each phenotypic group. Once scored, all pupae regardless of phenotype were placed in cages for the next generation. Both male and female pupae were kept in complete darkness for one week, when the adults emerged and completed mating, to reduce any competitive advantage provided by those individuals with wild-type eye pigmentation during mating, after which they were returned to the normal day/night light cycle.

TABLE 11
The multi-generation test for transgene elimination induced by the SSA trigger strains (G₄)

| | | Nos-I-SceI | | | | | | | | | |
|---|---|---|---|---|---|---|---|---|---|---|---|
| DSB repair-associated | | F₂ | | F₃ | | F₄ | | F₅ | | F₆ | |
| marker phenotypes^{a} | | ♂ | ♀ | ♂ | ♀ | ♂ | ♀ | ♂ | ♀ | ♂ | ♀ |
| | # Total pupae | 496 | 495 | 470 | 445 | 832 | 763 | 1215 | 1181 | 1222 | 1121 |
| No DSB | # WGR | 219 | 272 | 185 | 190 | 274 | 292 | 426 | 383 | 409 | 359 |
| | # WGRB | 155 | 119 | 153 | 142 | 274 | 227 | 314 | 332 | 325 | 280 |
| NHEJ | # WG | | 1 | 2 | | 1 | | 3 | 1 | 2 | 4 |
| | # WGB | | | 1 | 1 | | 1 | 15 | 11 | 12 | 10 |
| kmo^{Δ4} | # W | 51 | 63 | 63 | 54 | 138 | 123 | 207 | 241 | 225 | 238 |
| | # WB | 66 | 40 | 62 | 50 | 127 | 104 | 217 | 170 | 194 | 165 |
| SSA | # Blk | 2 | | 1 | 1 | 8 | 8 | 13 | 18 | 29 | 23 |
| | # BlkB | 3 | | | 5 | 3 | 6 | 10 | 8 | 12 | 16 |
| | # BlkGR | | | 2 | | 2 | 2 | 5 | 9 | 15 | 7 |
| | # BlkGRB | | | 1 | 2 | 5 | 3 | 5 | 10 | 7 | 18 |
| | # BlkG | | | | | | | | | | |
| | # BlkGB | | | | | | | | | | 1 |

| | | | | | | | | | | |
|---|---|---|---|---|---|---|---|---|---|---|
| % WG(WGR + WG + Blk) | 0 | 0.3 | 0.9 | 0.3 | 0.2 | 0.2 | 2.3 | 1.6 | 1.7 | 2 |
| % Blk(WGR + WG + Blk) | 1.3 | 0 | 1.2 | 2.4 | 3.2 | 3.2 | 4.2 | 5.6 | 6.9 | 9.1 |
| % WGR/Total | 75.4 | 79 | 72 | 74.6 | 65.9 | 68 | 60.9 | 60.5 | 60.1 | 57 |
| % BFP/Total | 45.2 | 32.1 | 46.2 | 45 | 49.2 | 44.7 | 46.2 | 45 | 45 | 43.7 |

| | | PUb-I-SceI | | | | | | | | | |
|---|---|---|---|---|---|---|---|---|---|---|---|
| DSB repair-associated | | F₂ | | F₃ | | F₄ | | F₅ | | F₆ | |
| marker phenotypes^{a} | | ♂ | ♀ | ♂ | ♀ | ♂ | ♀ | ♂ | ♀ | ♂ | ♀ |
| | # Total pupae | 479 | 563 | 495 | 439 | 977 | 927 | 1172 | 1149 | 976 | 849 |
| No DSB | # WGR | 292 | 318 | 242 | 184 | 402 | 325 | 447 | 450 | 435 | 275 |
| | # WGRB | 92 | 111 | 94 | 113 | 220 | 260 | 262 | 276 | 188 | 268 |
| NHEJ | # WGR | | | | 1 | | | | | | |
| | # WGB | | | | | | | | | | |
| kmo^{Δ4} | # W | 29 | 39 | 49 | 49 | 142 | 149 | 187 | 159 | 158 | 137 |
| | # WB | 66 | 95 | 107 | 90 | 210 | 191 | 274 | 261 | 188 | 164 |
| SSA | # Blk | 5 | | 3 | 2 | 1 | 1 | 2 | 3 | 4 | 1 |
| | # BlkB | | | | | 2 | 1 | | | 2 | 1 |
| | # BlkGR | | | | | | | | | | 1 |
| | # BlkGRB | | | | | | | | | 1 | 2 |
| | # BlkG | | | | | | | | | | |
| | # BlkGB | | | | | | | | | | |

| | | | | | | | | | | |
|---|---|---|---|---|---|---|---|---|---|---|
| % WG(WGR + WG + Blk) | 0 | 0 | 0 | 0.3 | 0 | 0 | 0 | 0 | 0 | 0 |
| % Blk(WGR + WG + Blk) | 1.3 | 0 | 0.9 | 0.7 | 0.5 | 0.3 | 0.3 | 0.4 | 1.1 | 0.9 |
| % WGR/Total | 80.2 | 76.2 | 67.9 | 67.7 | 63.7 | 63.1 | 60.5 | 63.2 | 63.8 | 64 |
| % BFP/Total | 33 | 36.6 | 40.6 | 46.2 | 44.2 | 48.8 | 45.7 | 46.7 | 38.8 | 51.2 |

^{a}Marker phenotypes: W, white eyes; Blk, black eyes; G, EGFP; R, DsRED; B, BFP.

TABLE 12
The multi-generation test for transgene elimination induced by the SSA trigger strains (G₁₂)

| DSB repair-associated | Nos-I-SceI | | | |
|---|---|---|---|---|
| marker phenotypes^{a} | F₂ | F₃ | F₄ | F₅ |

| | | | | | | | | | | | | | |
|---|---|---|---|---|---|---|---|---|---|---|---|---|---|
| | # Total pupae | 701 | 714 | 701 | 1951 | 1361 | 898 | 845 | 858 | 846 | 680 | 656 | 588 |
| No DSB | # WGR | 204 | 187 | 175 | 558 | 327 | 268 | 205 | 185 | 186 | 127 | 149 | 111 |
| | # WGRB | 299 | 345 | 343 | 898 | 639 | 372 | 430 | 377 | 388 | 229 | 261 | 188 |
| NHEJ | # WG | 1 | | | 4 | 2 | | 1 | | 2 | 2 | | |
| | # WGB | 3 | 2 | 3 | 22 | 8 | 4 | 10 | 4 | 9 | 9 | 8 | 6 |
| kmo^{Δ4} | # W | 56 | 35 | 49 | 116 | 83 | 55 | 27 | 61 | 57 | 19 | 38 | 31 |
| | # WB | 133 | 140 | 116 | 315 | 258 | 161 | 108 | 151 | 115 | 96 | 139 | 57 |
| SSA | # Blk | 1 | 1 | 2 | 7 | 8 | 2 | 5 | 1 | 10 | 11 | 5 | 32 |
| | # BlkB | 2 | 3 | 7 | 16 | 30 | 26 | 23 | 29 | 27 | 30 | 40 | 83 |
| | # BlkGR | 1 | | 3 | 6 | 3 | 8 | 8 | 4 | 14 | 38 | 7 | 37 |
| | # BlkGRB | 1 | 1 | 3 | 8 | 4 | 4 | 24 | 28 | 37 | 58 | 18 | 38 |
| | # BlkG | | | | | | | | | | 3 | | 1 |
| | # BlkGB | | | | 1 | 1 | | 4 | 2 | 1 | 10 | | 3 |

| | | | | | | | | | | | | |
|---|---|---|---|---|---|---|---|---|---|---|---|---|
| % WG(WGR + WG + Blk) | 0.78 | 0.37 | 0.56 | 1.71 | 0.96 | 0.6 | 1.6 | 0.6 | 1.6 | 2 | 1.6 | 1.2 |
| % Blk(WGR + WG + Blk) | 0.98 | 0.93 | 2.8 | 2.5 | 4.31 | 5.6 | 9 | 10 | 13 | 35 | 14 | 38.9 |
| % WGR/Total | 71.8 | 74.5 | 73.9 | 74.6 | 71 | 71 | 75 | 66 | 68 | 52 | 62 | 50.9 |
| % BFP/Total | 62.5 | 68.8 | 67.3 | 64.6 | 69.1 | 63 | 71 | 70 | 68 | 71 | 70 | 63.8 |

| DSB repair-associated | | PUb-I-SceI | | | | | | | | | | | |
|---|---|---|---|---|---|---|---|---|---|---|---|---|---|
| marker phenotypes^{a} | | F₂ | | | F₃ | | | F₄ | | | F₅ | | |
| | # Total pupae | 795 | 856 | 908 | 940 | 724 | 928 | 945 | 968 | 868 | 572 | 683 | 823 |
| No DSB | # WGR | 245 | 258 | 301 | 277 | 196 | 355 | 239 | 303 | 231 | 115 | 328 | 271 |
| | # WGRB | 357 | 389 | 397 | 382 | 330 | 348 | 352 | 462 | 366 | 149 | 218 | 230 |
| NHEJ | # WG | | | | | | | 1 | 1 | | | | |
| | # WGB | | | | 1 | | | | | | | | |
| kmo^{Δ4} | # W | 17 | 11 | 2 | 16 | 5 | 18 | 15 | 37 | 28 | 19 | 14 | 20 |
| | # WB | 176 | 198 | 208 | 196 | 179 | 180 | 170 | 158 | 175 | 72 | 112 | 154 |
| SSA | # Blk | 9 | 5 | 15 | 21 | 4 | 9 | 40 | 5 | 5 | 53 | | 28 |
| | # BlkB | | | | 25 | 1 | 1 | 39 | 1 | 29 | 141 | 4 | 54 |
| | # BlkGR | | | | 19 | 5 | 18 | 68 | 2 | 39 | 79 | 7 | 83 |
| | # BlkGRB | | | | 3 | 2 | 1 | 22 | | 4 | 44 | | 3 |
| | # BlkG | | | | | | | | | | | | |
| | # BlkGB | | | | | | | | | | | | |

| | | | | | | | | | | | | |
|---|---|---|---|---|---|---|---|---|---|---|---|---|
| % WG(WGR + WG + Blk) | 0 | 0 | 0 | 0.14 | 0 | 0 | 0.13 | 0.13 | 0 | 0 | 0 | 0 |
| % Blk(WGR + WG + Blk) | 0.82 | 0.77 | 2.1 | 9.34 | 2.22 | 3.7 | 22.2 | 1.03 | 10.2 | 54.6 | 2 | 21.6 |
| % WGR/Total | 75.7 | 75.6 | 76.9 | 78.1 | 72.9 | 75.8 | 62.5 | 79 | 68.8 | 39.3 | 79.9 | 60.9 |
| % BFP/Total | 67 | 68.6 | 66.6 | 64.6 | 71 | 57.1 | 61.6 | 64.2 | 66.1 | 60.4 | 48.9 | 54.8 |

^{a}Marker phenotypes: W, white eyes; Blk, black eyes; G, EGFP; R, DsRED; B, BFP.

For the Nos-I-SceI x kmo^{RG} experiment at the G₄ generation, five F₂ individuals with wild-type black eye (Blk) were identified from 765 WGR mosquitoes (0.7%), with the number of individuals with the restored phenotypes increasing by 10-fold when the experiment was concluded at F₆ (FIG. 30; Panel A and Table 11). To determine whether this increase was due to new SSA events each generation or to a selective advantage provided by the restoration of kmo, the same experiment was performed with PUb-I-SceI mosquitoes, with the addition of 5 wild-type individuals at the F₂ generation. No change in wild-type kmo allele frequency was observed in the PUb-I-SceI x kmo^{RG} experiment (FIG. 30; Panel A and Table 11), indicating the increase in wild-type, non-transgenic alleles in the nos-I-SceI experiment appeared to be due to SSA-based repair of I-SceI-induced DSBs and not to any competitive advantage of the wild-type over their white-eyed relatives. However, when this multi-generation SSA test was repeated at the G₁₂ generation, the frequencies of black-eyed individuals in the spike-in control cage populations were more variable (2-10 fold), and appeared to depend on the starting frequency in each individual cage at every generation (FIG. 30; Panel C and Table 12). These results confirm that SSA can generate a sufficient number of wild-type individuals to allow selection to act.

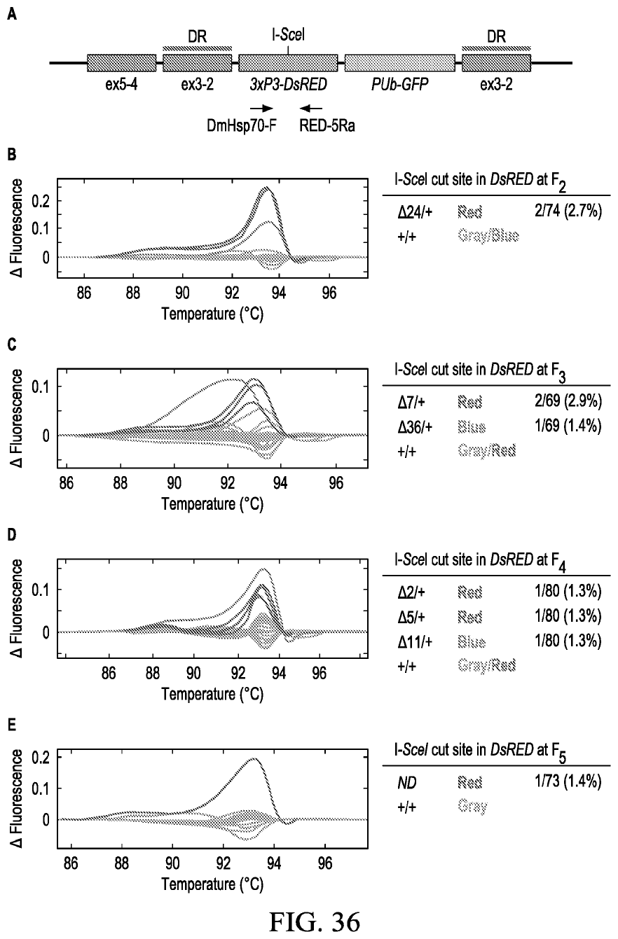

Interestingly, the frequency of restored wild-type individuals increased much faster in the G₁₂ experiment (30-40% at F₅) as compared to the G₄ experiment (less than 10% at F₆). One potential explanation for this is due to greater exposure to the I-SceI nuclease (avg. 44.2% in G₄; avg. 67% in G₁₂), indicating that the rate of DSB induction and hence repair would likely be even higher with the I-SceI transgene encoded at the target locus itself, generating a complete self-eliminating transgene. Once again, compared to SSA-associated alleles (Blk), NHEJ-driven indels in DsRED (WG) occurred at lower frequencies (Avg. ˜1%) (FIG. 30; Panels A and C, Table 11, and Table 12) and while these events were identified in the WGR group every generation, they did not increase over time (FIG. 36). Taken together, nos-driven I-SceI expression can reliably induce the removal of transgene sequences, and the resulting SSA-repair can faithfully restore the disrupted gene in Aedes aegypti. Furthermore, the core molecular elements of SSA, two flanking direct repeats and a transgene-specific DSB, are effective for erasing transgenes from a GM mosquito strain. Interestingly, the transgene-inserted allele can be restored flawlessly and thereby rescue the wild-type genotype and/or phenotype; and this seamless recovery of the targeted gene increased across multiple generations by nos-driven germline-specific SSA activation. As SSA-based repair is shared by diverse organisms; Drosophila melanogaster, Aedes aegypti, Saccharomyces cerevisiae, Arabidopsis thaliana, Caenorhabditis elegans, and mammalian cells, this rescue technology is expected to be amenable for potentially broad applications with a species-specific, spatial-temporal activation control

### Example 32: A Single Component System SSA-Based Rescue Strategy

Genetic control strategies have significant promise to prevent the transmission of highly pathogenic diseases by rapidly spreading beneficial genetic traits such as pathogen-resistance into vector mosquito populations. However, the highly invasive, self-propagating nature of gene drive-based transgene-delivery systems presents a challenge for conducting field trials and the ultimate development of gene drive technologies. The invasive nature of gene drive approaches heighten concerns related to releasing genetically modified organisms (GMOs), in terms of both health and ecological safety. Along with institutional containment protocols and field test-related governance and guidance of gene drive-modified insects, novel confinable gene drive strategies have been suggested to eliminate unwanted invasion to non-target populations. However, many such approaches only aim to halt the process of gene drive and do not directly delete the transgene itself from gene drive mosquitoes or they fail to restore the wild-type gene.

The rates of successful transgene removal via SSA as described are anticipated to be sufficient to counteract homing-based gene drive approaches. In certain embodiments, an SSA-based rescue strategy will be designed to remove transgenic material in the targeted population by both removing the effector gene while simultaneously restoring a wild-type allele from the gene drive allele. For example, a single component system consisting of both a homing-based gene drive and an SSA-based self-elimination mechanism at a single locus is predicted to allow the temporary invasion of a gene drive transgene (allowing potential field testing), with SSA-triggered reversion to wild-type occurring with no need for remediation such as the inundated release of wild-type strains. SSA-based transgenes could also be incorporated into split drive or daisy-chain drive approaches that utilizes composite interactions of multiple transgenes, potentially shortening the lifespan of each component.

What is claimed is:

1. A recombinant polynucleotide construct comprising direct repeat sequences flanking a DNA sequence comprising a transgene and at least a first site-specific nuclease recognition site.

2. The polynucleotide construct of claim 1, wherein the DNA sequence comprises said first site-specific nuclease recognition site and a second site-specific nuclease recognition site flanking said transgene.

3. The polynucleotide construct of claim 2, wherein the first and second site-specific nuclease recognition site are the same.

4. The polynucleotide construct of claim 2, wherein the first and second site-specific nuclease recognition site are different.

5. The polynucleotide construct of claim 1, wherein the site-specific nuclease recognition site is recognized by an engineered nuclease.

6. The polynucleotide construct of claim 1, wherein the site-specific nuclease recognition site is recognized by a nuclease native to at least a first eukaryotic species.

7. The polynucleotide construct of claim 1, wherein the DNA sequence comprises a reporter gene.

8. The polynucleotide construct of claim 1, wherein the direct repeat sequences comprise from about 2 to about 200 repeats.

9. The polynucleotide construct of claim 1, wherein the direct repeat sequences comprise from about 15 to about 20000 nucleotides.

10. The polynucleotide construct of claim 1, further comprising a selectable marker.

11. The polynucleotide construct of claim 1, further comprising a nucleic acid sequence encoding a nuclease that recognizes said site-specific nuclease recognition site.

12. The polynucleotide construct of claim 11, wherein said nucleic acid sequence is operably linked to an inducible or tissue-specific promoter.

13. The polynucleotide construct of claim 12, wherein said tissue-specific promoter is a germline-specific promoter.

14. The polynucleotide construct of claim 11, further comprising a second nucleic acid sequence encoding a second nuclease that recognizes a second site-specific nuclease recognition site in said DNA sequence.

15. The polynucleotide construct of claim 14, wherein the first and second nucleic acid sequences are operably linked to different promoters that drive different levels of expression.

16. A host cell comprising the polynucleotide construct of claim 1.

17. A transgenic plant, insect or non-human animal comprising the polynucleotide construct of claim 1, wherein said transgene is capable of being eliminated in progeny of said plant, insect or non-human animal.

18. A method of transforming a host cell comprising introducing the polynucleotide construct of claim 1 into said cell.

19. A method of eliminating a transgene sequence from a cell comprising subjecting a cell according to claim 16 to an external stimulus that causes the transgene sequence to be eliminated.

20. The method of claim 19, wherein the external stimulus is a chemical stimulus.
